(19) United States
(12) Patent Application Publication
(10) Pub. No.: US 2011/0046131 A1
(43) Pub. Date: Feb. 24, 2011

(54) PURINES AS PKC-THETA INHIBITORS

(75) Inventors: Irina Neagu, Belmont, MA (US); Andrew Laird Roughton, Plainsboro, NJ (US); Koc-Kan Ho, West Windsor, NJ (US); David Diller, East Windsor, NJ (US); Jui-Hsiang Chan, West Windsor, NJ (US); Michael Ohlmeyer, Plainsboro, NJ (US); Celia Kingsbury, Cream Ridge, NJ (US); Johannes Petrus Maria Lommerse, Oss (NL); Neeltje Miranda, Oss (NL); Jacobus Cornelis Henricus Maria Wijkmans, Oss (NL)

Correspondence Address: ORGANON USA, INC.;c/o MERCK, 2000 Galloping Hill Road, Mail Stop: K-6-1, 1990, Kenilworth, NJ, 07033, US

(73) Assignee: N.V. Organon and Pharmacopeia, LLC

(21) Appl. No.: 12/445,862

(22) PCT Filed: Oct. 19, 2007
(86) PCT No.: PCT/US07/81899

§ 371 Date: Nov. 10, 2010

Related U.S. Application Data
(60) Provisional application No. 60/853,396, filed on Oct. 20, 2006.

(51) Int. Cl. A61K 31/5377; C07D 473/32; A61K 31/52; A61P 11/06; A61P 29/00; A61P 37/06; A61P 25/00; A61P 19/02; A61P 35/00; A61P 3/10

(52) U.S. Cl. 514/234.2; 544/277; 544/118; 514/263.22; 514/263.2; 514/252.16; 514/263.4

(57) ABSTRACT

A chemical genus of purines, which are useful as PKCθ inhibitors, is disclosed. The genus is represented by the formula (I); A representative example is: (II)

### FIELD OF THE INVENTION

The present invention relates to a chemical genus of purines which are useful as PKCθ inhibitors.

### BACKGROUND OF THE INVENTION

Members of the protein kinase C (PKC) family of serine/threonine kinases play critical roles in the regulation of cellular differentiation and proliferation of diverse cell types. Ten mammalian members of PKC family have been identified and designated α, β, γ, δ, ε, ζ, η, θ, μ, and λ. The structure of PKCθ displays the highest homology with members of the Ca²⁺ independent novel PKC subfamily, including PKCδ, ε, and η. PKCθ is most highly related to PKCδ.

PKCθ is expressed predominantly in lymphoid tissue and skeletal muscle. It has been shown that PKCθ is essential for TCR-mediated T-cell activation but inessential during TCR-dependent thymocyte development. PKCθ, but not other PKC isoforms, translocates to the site of cell contact between antigen-specific T-cells and APCs, where it localizes with the TCR in the central core of the T-cell activation. PKCθ, but not the α, ε, or ζ isoenzymes, selectively activated a FasL promoter-reporter gene and upregulated the mRNA or cell surface expression of endogenous FasL. On the other hand, PKCθ and ε promoted T-cell survival by protecting the cells from Fas-induced apoptosis, and this protective effect was mediated by promoting p90Rsk-dependent phosphorylation of BAD. Thus, PKCθ appears to play a dual regulatory role in T-cell apoptosis.

The selective expression of PKCθ in T-cells and its essential role in mature T-cell activation establish that PKCθ inhibitors are useful for the treatment or prevention of disorders or diseases mediated by T lymphocytes, for example, autoimmune disease such as rheumatoid arthritis and lupus erythematosus, and inflammatory disease such as asthma and inflammatory bowel diseases.

PKCθ is identified as a drug target for immunosuppression in transplantation and autoimmune diseases (Isakov et al. (2002) Annual Review of Immunology, 20, 761-794). PCT Publication WO2004/043386 identifies PKCθ as a target for treatment of transplant rejection and multiple sclerosis. PKCθ also plays a role in inflammatory bowel disease (The Journal of Pharmacology and Experimental Therapeutics (2005), 313 (3), 962-982), asthma (WO 2005062918), and lupus (Current Drug Targets: Inflammation & Allergy (2005), 4 (3), 295-298).

In addition, PKCθ is highly expressed in gastrointestinal stromal tumors (Blay, P. et al. (2004) Clinical Cancer Research, 10, 12, Pt. 1), it has been suggested that PKCθ is a molecular target for treatment of gastrointestinal cancer (Wiedmann, M. et al. (2005) Current Cancer Drug Targets 5(3), 171). Thus, small molecule PKC-theta inhibitors can be useful for treatment of gastrointestinal cancer.

Experiments conduced in PKCθ knock-out mice led to the conclusion that PKCθ inactivation prevented fat-induced defects in insulin signalling and glucose transport in skeletal muscle (Kim J. et al, 2004, The J. of Clinical Investigation 114 (6), 823). This data suggests that PKCθ is a potential therapeutic target for the treatment of type 2 diabetes, and hence small molecule PKCθ inhibitors can be useful for treating such disease.

Therefore, PKCθ inhibitors are useful in treatment of T-cell mediated diseases including autoimmune disease such as rheumatoid arthritis and lupus erythematosus, and inflammatory diseases such as asthma and inflammatory bowel disease. In addition, PKCθ inhibitors are useful in treatment of gastrointestinal cancer and diabetes.

Japanese application number 2003-008019, published on Aug. 5, 2004 under publication number JP 2004-217582, discloses purine derivatives having alleged utility as TNA-alpha production inhibitors and PDE4 inhibitors.

### SUMMARY OF THE INVENTION

In one aspect, the invention relates to compounds of the formula I:

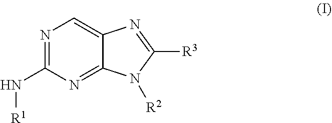

wherein:
R¹ is chosen from C₁-C₄ alkyl, carbocyclyl, substituted carbocyclyl and

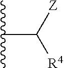

wherein
R⁴ is chosen from cycloalkyl, cycloalkylalkyl, aryl, arylalkyl, heteroaryl, and heteroarylalkyl, wherein R⁴ may be substituted, with a proviso that when R⁴ is a heteroaryl, R⁴ is not bonded via a heteroatom to the methylene carbon bearing the Z group; and
Z is chosen from —H and C₁-C₄ alkyl;
R² is chosen from —(C₂-C₇ alkyl)-NR⁵R⁶, —(C₀-C₄ alkyl)-R⁷-R⁸, and —(C₀-C₄ alkyl)-C(O)—(C₀-C₄ alkyl)-R⁷-R⁸,
   wherein
   R⁷ is cyclyl,
      with a proviso that when R⁷ is a heterocyclyl, a purine nitrogen of Formula I bonded to R² is not bonded to a heteroatom of R⁷ directly or via a methylene group;
   R⁸ is chosen from —(C₀-C₄ alkyl)-NR⁵R⁶, and
      —C(O)—(C₀-C₄ alkyl)-NR⁵R⁶, and, when R⁷ is nitrogenous heterocyclyl, R⁸ may additionally be —H,
      with a proviso that when R⁷ is a heterocyclyl and R⁸ is —(C₀-C₄ alkyl)-NR⁵R⁶, a heteroatom of R⁷ is not bonded to —NR⁵R⁶ directly or via a methylene group;
   R⁵ and R⁶ are independently chosen from —H and C₁-C₄ alkyl; and
R³ is chosen from C₁-C₆ alkyl, aryl, substituted aryl, arylalkyl, substituted arylalkyl, heteroaryl and substituted heteroaryl;
   with a proviso that when R³ is phenyl and R² is piperidin-4-yl-ethyl, R¹ is not cyclopropyl.

In another aspect the invention relates to pharmaceutical compositions comprising a pharmaceutically acceptable carrier and a compound of formula I, or salt thereof.

In another aspect the invention relates to a method for treating T-cell mediated diseases including autoimmune disease such as rheumatoid arthritis and lupus erythematosus, inflammatory diseases such as asthma and inflammatory bowel disease, cancer such as gastrointestinal cancer, and diabetes. The method comprises administering a therapeutically effective amount of a compound of formula I, or salt thereof.

### DETAILED DESCRIPTION OF THE INVENTION

In its broadest sense, the invention relates to compounds of the formula I, or salt thereof:

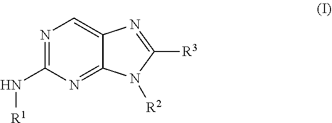

wherein:
R¹ is chosen from C₁-C₄ alkyl, carbocyclyl, substituted carbocyclyl and

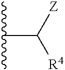

wherein
R⁴ is chosen from cycloalkyl, cycloalkylalkyl, aryl, arylalkyl, heteroaryl, and heteroarylalkyl, wherein R⁴ may be substituted, with a proviso that when R⁴ is a heteroaryl, R⁴ is not bonded via a heteroatom to the methylene carbon bearing the Z group; and
Z is chosen from —H and C₁-C₄ alkyl;
R² is chosen from —(C₂-C₇ alkyl)-NR⁵R⁶, —(C₀-C₄ alkyl)-R⁷-R⁸, and —(C₀-C₄ alkyl)-C(O)—(C₀-C₄ alkyl)-R⁷-R⁸,
   wherein
   R⁷ is cyclyl,
      with a proviso that when R⁷ is a heterocyclyl, a purine nitrogen of Formula I bonded to R² is not bonded to a heteroatom of R⁷ directly or via a methylene group;
   R⁸ is chosen from —(C₀-C₄ alkyl)-NR⁵R⁶, and
      —C(O)—(C₀-C₄ alkyl)-NR⁵R⁶, and, when R⁷ is nitrogenous heterocyclyl, R⁸ may additionally be —H,
      with a proviso that when R⁷ is a heterocyclyl and R⁸ is —(C₀-C₄ alkyl)-NR⁵R⁶, a heteroatom of R⁷ is not bonded to —NR⁵R⁶ directly or via a methylene group;
   R⁵ and R⁶ are independently chosen from —H and C₁-C₄ alkyl; and
R³ is chosen from C₁-C₆ alkyl, aryl, substituted aryl, arylalkyl, substituted arylalkyl, heteroaryl and substituted heteroaryl; with a proviso that when R³ is phenyl and R² is piperidin-4-yl-ethyl, R¹ is not cyclopropyl.

In one embodiment, R¹ is chosen from C₁-C₄ alkyl, phenyl optionally substituted with one or two substituents independently chosen from halogen, OCH₃, —CF₃, —OCF₃ and C₁-C₄ alkyl,

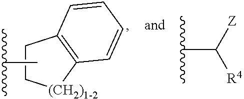

wherein
R⁴ is —(C₀-C₄ alkyl)-R⁹,
   wherein
   R⁹ is chosen from cycloalkyl, aryl, and heteroaryl, wherein R⁹ is optionally substituted at one or two atoms with substituents independently chosen from halogen, —OH, —OCH₃, —CF₃, —OCF₃, —CN, C₁-C₄ alkyl, and pyridinyl; and
Z is chosen from —H and C₁-C₄ alkyl.

In another embodiment, R² is chosen from —(C₂-C₇ alkyl)-NR⁵R⁶, —(C₀-C₄ alkyl)-R⁷-R⁸, and —(C₀-C₄ alkyl)-C(O)—(C₀-C₄ alkyl)-R⁷-R⁸,
wherein
R⁷ is chosen from alicyclyl, nitrogenous alicyclyl, aryl, and nitrogenous heteroaryl;
R⁸ is chosen from —H, —(C₀-C₄ alkyl)-NR⁵R⁶, and —C(O)—(C₀-C₄ alkyl)-NR⁵R⁶; and
R⁵ and R⁶ are independently chosen from —H and —(C₁-C₄ alkyl).

In another embodiment, R³ is chosen from C₁-C₆ alkyl, aryl, aryl substituted with R¹⁰, R¹¹ and R¹²,
wherein
R¹⁰, R¹¹ and R¹² are independently chosen from —H, halogen, —OCH₃, —CF₃, —OCF₃, —CN, C₁-C₄ alkyl, —NR¹³R¹⁴, —S(O)ₘCH₃, —CONHR²², —NHCOR²³, —OR²⁴ and —NHS(O)ₘR²⁵;
wherein
R¹³ and R¹⁴ are independently chosen from —H and C₁-C₄ alkyl;
R²², R²³ and R²⁴ are one or two substituents independently chosen from —H, C₁-C₄ alkyl, C₁-C₆ cycloalkyl, aryl, —(CH₂)—NR²⁶R²⁷ and —(CH₂)ₙOR²⁸ said C₁-C₄ alkyl and C₁-C₆ cycloalkyl being optionally substituted with one or more halogens;
R²⁵ is C₁-C₄ alkyl;
R²⁶ and R²⁷ are independently chosen from H and C₁-C₄ alkyl or
R²⁶ and R²⁷ with the N to which they are attached form a 4-7 membered saturated heterocyclic ring optionally comprising an O;
R²⁸ is chosen from H and C₁-C₄ alkyl;
m is 0, 1 or 2 and
n is 1, 2 or 3.

In another embodiment, R¹ is chosen from C₁-C₄ alkyl, phenyl optionally substituted with one or two substituents independently chosen from halogen, OCH₃, —CF₃, —OCF₃ and C₁-C₄ alkyl,

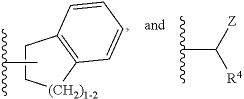

wherein
R⁴ is chosen from

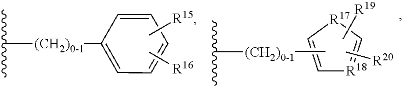

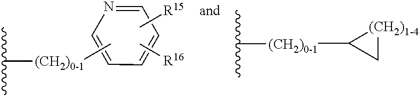

wherein
R¹⁵ and R¹⁶ are independently chosen from —H, halogen, —OH, —OCH₃, —CF₃, —OCF₃, —CN, C₁-C₄ alkyl, and pyridinyl;
R¹⁷ is chosen from O and S;
R¹⁸ is chosen from CH and N;
R¹⁹ and R²⁰ are independently chosen from —H, halogen, —OCH₃, —CF₃, —OCF₃, —CN, C₁-C₄ alkyl, and pyridinyl; and
Z is chosen from —H and C₁-C₄ alkyl.

In another embodiment, R² is chosen from —(C₂-C₇ alkyl)-NR⁵R⁶, —(C₀-C₄ alkyl)-R⁷-R⁸, and —(C₀-C₄ alkyl)-C(O)—(C₀-C₄ alkyl)-R⁷-R⁸,
wherein
R⁷ is chosen from cyclohexyl, phenyl, piperidinyl, pyrrolidinyl, morpholinyl, and piperazinyl;
R⁸ is chosen from —H, —(C₀-C₄ alkyl)-NR⁵R⁶, and —C(O)—(C₀-C₄ alkyl)-NR⁵R⁶; and
R⁵ and R⁶ are independently chosen from —H and —(C₁-C₄ alkyl).

In another embodiment, R² is other than

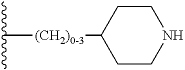

and

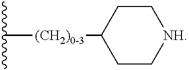

In another embodiment, R³ is chosen from C₁-C₆ alkyl,

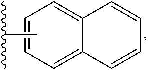

and

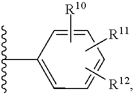

wherein
R¹⁰, R¹¹ and R¹² are independently chosen from —H, halogen, —OCH₃, —CF₃, —OCF₃, —CN, C₁-C₄ alkyl, —NR¹³R¹⁴, —S(O)ₘCH₃, —CONHR²², —NHCOR²³, —OR²⁴ and —NHS(O)ₘR²⁵;
wherein
R¹³ and R¹⁴ are independently chosen from —H and C₁-C₄ alkyl;
R²², R²³ and R²⁴ are one or two substituents independently chosen from —H, C₁-C₄ alkyl, C₁-C₆ cycloalkyl, aryl, —(CH₂)ₙNR²⁶R²⁷ and —(CH₂)ₙOR²⁸ said C₁-C₄ alkyl and C₁-C₆ cycloalkyl being optionally substituted with one or more halogens;
R²⁵ is C₁-C₄ alkyl;
R²⁶ and R²⁷ are independently chosen from H and C₁-C₄ alkyl or
R²⁶ and R²⁷ with the N to which they are attached form a 4-7 membered saturated heterocyclic ring optionally comprising an O;
R²⁸ is chosen from H and C₁-C₄ alkyl;
m is 0, 1 or 2 and
n is 1, 2 or 3.

In another embodiment R³ is chosen from pyridyl, thienyl, thiazolyl and furanyl optionally substituted with methyl or halogen.

In a different embodiment, the invention relates to compounds of the formula I, or salt thereof:

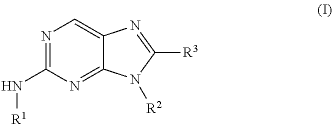

wherein:
R¹ is chosen from straight or branched C₁-C₄ alkyl, phenyl optionally substituted with one or two substituents independently chosen from halogen, OCH₃, —CF₃, —OCF₃ and C₁-C₄ alkyl,

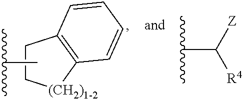

wherein
R⁴ is —(C₀-Cₐ alkyl)-R⁹;
   wherein
   R⁹ is chosen from cycloalkyl, aryl, and heteroaryl,
      wherein R⁹ is optionally substituted at one or two atoms with substituents independently chosen from halogen, —OH, —OCH₃, —CF₃, —OCF₃, —CN, C₁-C₄ alkyl, and pyridinyl,
      with a proviso that when R⁹ is a heteroaryl, R⁹ is not bonded via a heteroatom to the methylene carbon bearing the Z group; and
Z is chosen from —H and C₁-C₄ alkyl;
R² is chosen from —(C₂-C₇ alkyl)-NR⁵R⁶, —(C₀-C₄ alkyl)-R⁷-R⁸, and —(C₀-C₄ alkyl)-C(O)—(C₀-C₄ alkyl)-R⁷-R⁸, wherein
   R⁷ is chosen from alicyclyl, nitrogenous alicyclyl, aryl, and nitrogenous heteroaryl,
      with a proviso that when R⁷ is a nitrogenous alicyclyl or a nitrogenous heteroaryl, a purine nitrogen of Formula I bonded to R² is not bonded directly or via a methylene group to a nitrogen of R⁷;
   R⁸ is chosen from, —(C₀-C₄ alkyl)-NR⁵R⁶, and —C(O)—(C₀-C₄ alkyl)-NR⁵R⁶, and, when R⁷ is nitrogenous alicyclyl or nitrogenous heteroaryl, R⁸ may additionally be —H,
      with a proviso that when R⁷ is a nitrogenous alicyclyl or a nitrogenous heteroaryl and R⁸ is —(C₀-C₄ alkyl)-NR⁵R⁶, a nitrogen of R⁷ is not bonded directly or via a methylene group to —NR⁵R⁶; and
   R⁵ and R⁶ are independently chosen from —H and —(C₁-C₄ alkyl);
R³ is chosen from C₁-C₆ alkyl, aryl, aryl substituted with R¹⁰, R¹¹ and R¹², wherein
   R¹⁰, R¹¹ and R¹² are independently chosen from —H, halogen, —OCH₃, —CF₃, —OCF₃, —CN, C₁-C₄ alkyl, —NR¹³R¹⁴, —S(O)ₘCH₃, —CONHR²², —NHCOR²³, —OR²⁴ and —NHS(O)ₘR²⁵;
   wherein
   R¹³ and R¹⁴ are independently chosen from —H and C₁-C₄ alkyl;
   R²², R²³ and R²⁴ are one or two substituents independently chosen from —H, C₁-C₄ alkyl, C₁-C₆ cycloalkyl, aryl, —(CH₂)ₙNR²⁶R²⁷ and —(CH₂)ₙOR²⁸ said C₁-C₄ alkyl and C₁-C₆ cycloalkyl being optionally substituted with one or more halogens;
   R²⁵ is C₁-C₄ alkyl;
   R²⁶ and R²⁷ are independently chosen from H and C₁-C₄ alkyl or
   R²⁶ and R²⁷ with the N to which they are attached form a 4-7 membered saturated heterocyclic ring optionally comprising an O;
   R²⁸ is chosen from H and C₁-C₄ alkyl;
   m is 0, 1 or 2 and
   n is 1, 2 or 3

In one embodiment, R¹ is chosen from C₁-C₄ alkyl, phenyl optionally substituted with one or two substituents independently chosen from halogen, OCH₃, —CF₃, —OCF₃ and C₁-C₄ alkyl,

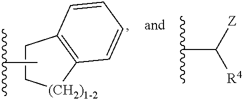

wherein
R⁴ is chosen from

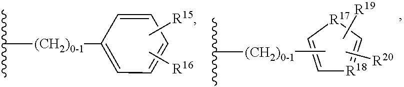

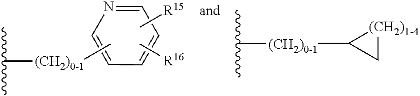

wherein
R¹⁵ and R¹⁶ are independently chosen from —H, halogen, —OH, —OCH₃, —CF₃, —OCF₃, —CN, C₁-C₄ alkyl, and pyridinyl;
R¹⁷ is chosen from O and S;
R¹⁸ is chosen from CH and N;
R¹⁹ and R²⁹ are independently chosen from —H, halogen, —OCH₃, —CF₃, —OCF₃, —CN, C₁-C₄ alkyl, and pyridinyl; and
Z is chosen from —H and C₁-C₄ alkyl.

In one embodiment, R² is chosen from —(C₂-C₇ alkyl)-NR⁵R⁶, —(C₀-C₄ alkyl)-R⁷-R⁸, and —(C₀-C₄ alkyl)-C(O)—(C₀-C₄ alkyl)-R⁷-R⁸,
wherein
R⁷ is chosen from cyclohexyl, phenyl, piperidinyl, pyrrolidinyl, morpholinyl, and piperazinyl;
R⁸ is chosen from —H, —(C₀-C₄ alkyl)-NR⁵R⁶, and —C(O)—(C₀-C₄ alkyl)-NR⁵R⁶; and
R⁵ and R⁶ are independently chosen from —H and —(C₁-C₄ alkyl).

In another embodiment, R² is other than

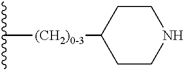

and

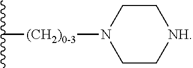

In another embodiment, R³ is chosen from C₁-C₆ alkyl,

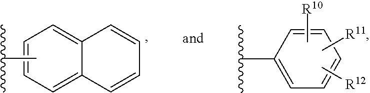

wherein
R¹⁰, R¹¹ and R¹² are independently chosen from —H, halogen, —OCH₃, —CF₃, —OCF₃, —CN, C₁-C₄ alkyl, —NR¹³R¹⁴, —S(O)ₘCH₃, —CONHR²², —NHCOR²³, —OR²⁴ and —NHS(O)ₘR²⁵;
wherein
R¹³ and R¹⁴ are independently chosen from —H and C₁-C₄ alkyl;
R²², R²³ and R²⁴ are one or two substituents independently chosen from —H, C₁-C₄ alkyl, C₁-C₆ cycloalkyl, aryl, —(CH₂)ₙNR²⁶R²⁷ and —(CH₂)ₙOR²⁸ said C₁-C₄ alkyl and C₁-C₆ cycloalkyl being optionally substituted with one or more halogens;
R²⁵ is C₁-C₄ alkyl;
R²⁶ and R²⁷ are independently chosen from H and C₁-C₄ alkyl or
R²⁶ and R²⁷ with the N to which they are attached form a 4-7 membered saturated heterocyclic ring optionally comprising an O;
R²⁸ is chosen from H and C₁-C₄ alkyl;
m is 0, 1 or 2 and
n is 1, 2 or 3.

In another embodiment R³ is chosen from pyridyl, thienyl, thiazolyl and furanyl optionally substituted with methyl or halogen

In another embodiment, R¹ is

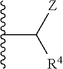

wherein
R⁴ is chosen from

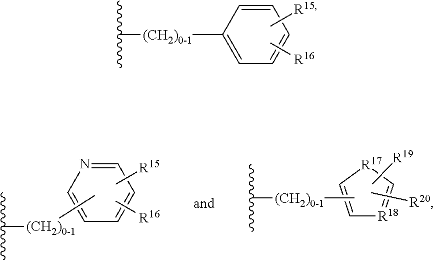

wherein
R¹⁵ and R¹⁶ are independently chosen from —H, halogen, —OH, —OCH₃, —CF₃, —OCF₃, —CN and C₁-C₄ alkyl;
R¹⁷ is chosen from O and S;
R¹⁸ is chosen from CH and N;
R¹⁹ and R²⁰ are independently chosen from —H, halogen, —OCH₃, —CF₃, —OCF₃, —CN, C₁-C₄ alkyl, and pyridinyl; and
Z is chosen from —H and C₁-C₄ alkyl.

In another embodiment, R² is chosen from

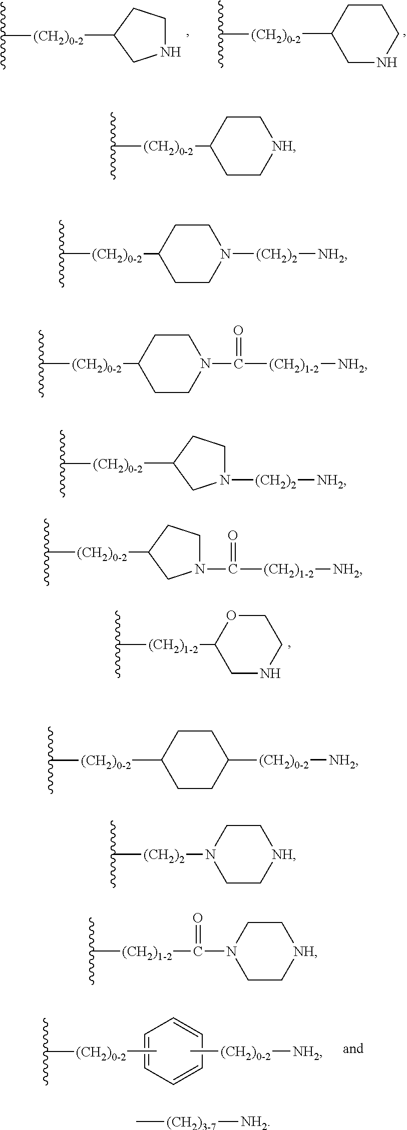

In another embodiment, R² is chosen from

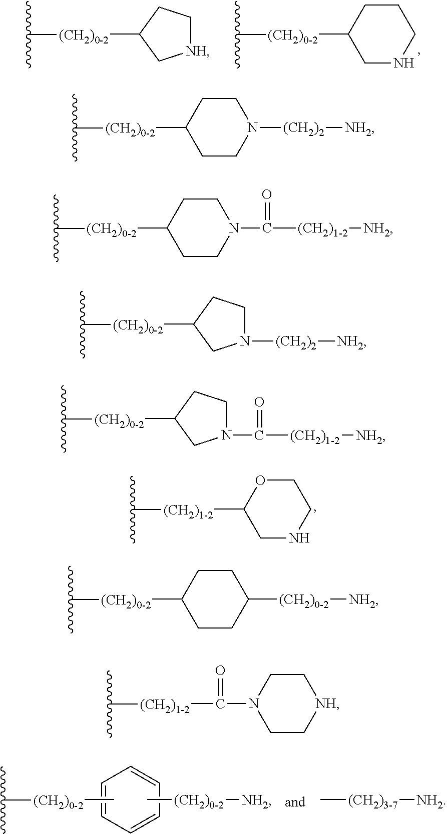

In another embodiment, R³ is

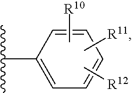

wherein
R¹⁰, R¹¹ and R¹² are independently chosen from —H, halogen, —OCH₃, —CF₃, —OCF₃, —CN, C₁-C₄ alkyl, —NR¹³R¹⁴, —S(O)ₘCH₃, —CONHR²², —NHCOR²³, —OR²⁴ and —NHS(O)ₘR²⁵;
wherein
R¹³ and R¹⁴ are independently chosen from —H and C₁-C₄ alkyl;
R²², R²³ and R²⁴ are one or two substituents independently chosen from —H, C₁-C₄ alkyl, C₁-C₆ cycloalkyl, aryl, —(CH₂)ₙNR²⁶R²⁷ and —(CH₂)—OR²⁸ said C₁-C₄ alkyl and C₁-C₆ cycloalkyl being optionally substituted with one or more halogens;
R²⁵ is C₁-C₄ alkyl;
R²⁶ and R²⁷ are independently chosen from H and C₁-C₄ alkyl or
R²⁶ and R²⁷ with the N to which they are attached form a 4-7 membered saturated heterocyclic ring optionally comprising an O;
R²⁸ is chosen from H and C₁-C₄ alkyl;
m is 0, 1 or 2 and
n is 1, 2 or 3.

In another embodiment R³ is chosen from pyridyl, thienyl, thiazolyl and furanyl optionally substituted with methyl or halogen.

In yet another embodiment, the invention relates to compounds of the formula I, or salt thereof:

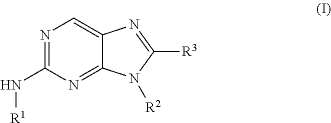

wherein:
R¹ is chosen from C₁-C₄ alkyl, phenyl optionally substituted with one or two substituents independently chosen from halogen, OCH₃, —CF₃, —OCF₃ and C₁-C₄ alkyl,

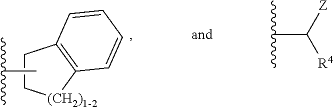

wherein
R⁴ is chosen from

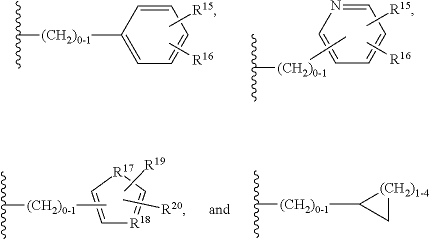

wherein
R¹⁵ and R¹⁶ are independently chosen from —H, halogen, —OH, —OCH₃, —CF₃, —OCF₃, —CN, C₁-C₄ alkyl, and pyridinyl;
R¹⁷ is chosen from O and S;
R¹⁸ is chosen from CH and N;
R¹⁹ and R²⁰ are independently chosen from —H, halogen, —OCH₃, —CF₃, —OCF₃, —CN, C₁-C₄ alkyl, and pyridinyl; and
Z is chosen from —H and C₁-C₄ alkyl;
R² is chosen from —(C₂-C₇ alkyl)-NR⁵R⁶, —(C₀-Cₐ alkyl)-R⁷-R⁸, and —(C₀-C₄ alkyl)-C(O)—(C₀-C₄ alkyl)-R⁷-R⁸,
   wherein
   R⁷ is chosen from cyclohexyl, phenyl, piperidinyl, pyrrolidinyl, morpholinyl, and piperazinyl;
   R⁸ is chosen from —H, —(C₀-C₄ alkyl)-NR⁵R⁶, and —C(O)—(C₀-C₄ alkyl)-NR⁵R⁶; and
R⁵ and R⁶ are independently chosen from —H and —(C₁-C₄ alkyl);
   and R² contains a basic N atom located from 2 to 8 atoms distant from its point of attachment to the purine ring;
R³ is chosen from C₁-C₆ alkyl,

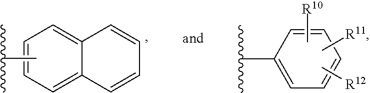

wherein
R¹⁰, R¹¹ and R¹² are independently chosen from —H, halogen, —OCH₃, —CF₃, —OCF₃, —CN, C₁-C₄ alkyl, —NR¹³R¹⁴, —S(O)ₘCH₃, —CONHR²², —NHCOR²³, —OR²⁴ and —NHS(O)ₘR²⁵;
wherein
R¹³ and R¹⁴ are independently chosen from —H and C₁-C₄ alkyl;
R²², R²³ and R²⁴ are one or two substituents independently chosen from —H, C₁-C₄ alkyl, C₁-C₆ cycloalkyl, aryl, —(CH₂)ₙNR²⁶R²⁷ and —(CH₂)ₙOR²⁸ said C₁-C₄ alkyl and C₁-C₆ cycloalkyl being optionally substituted with one or more halogens;
R²⁵ is C₁-C₄ alkyl;
R²⁶ and R²⁷ are independently chosen from H and C₁-C₄ alkyl or
R²⁶ and R²⁷ with the N to which they are attached form a 4-7 membered saturated heterocyclic ring optionally comprising an O;
R²⁸ is chosen from H and C₁-C₄ alkyl;
m is 0, 1 or 2 and
n is 1, 2 or 3

In another embodiment R³ is chosen from pyridyl, thienyl, thiazolyl and furanyl optionally substituted with methyl or halogen.

When reference is made to a basic N atom, such N atom has a lone pair of electrons available for protonation. N atoms with a basicity below pK_{b} of about 9 are preferred. More preferred are N atoms which exhibit pK_{b} below 7. Such basic N atom may be primary, secondary, or tertiary amine, in linear, branched or cyclic system. Examples of R² containing basic N atom located from 2 to 8 atoms distant from its point of attachment to the purine ring are:

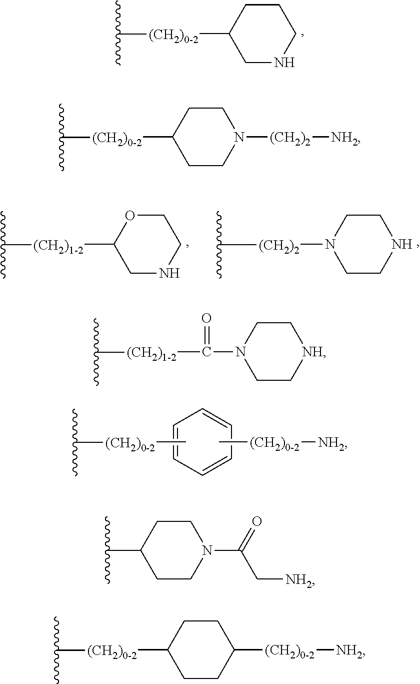

### —(CH₂)₃₋₇—NH₂, —(CH₂)₃₋₇—NH(CH₃), and —(CH₂)₃₋₇—N(CH₃)₂.

In one embodiment, R¹ is

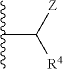

wherein
R⁴ is chosen from

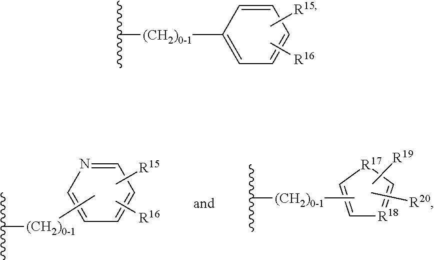

wherein
R¹⁵ and R¹⁶ are independently chosen from —H, halogen, —OH, —OCH₃, —CF₃, —OCF₃, —CN and C₁-C₄ alkyl;
R¹⁷ is chosen from O and S;
R¹⁸ is chosen from CH and N;
R¹⁹ and R²⁰ are independently chosen from —H, halogen, —OCH₃, —CF₃, —OCF₃, —CN, C₁-C₄ alkyl, and pyridinyl; and
Z is chosen from —H and C₁-C₄ alkyl.

In another embodiment, R² is not

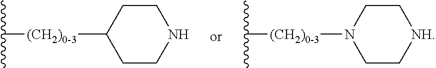

In another embodiment, R² is chosen from

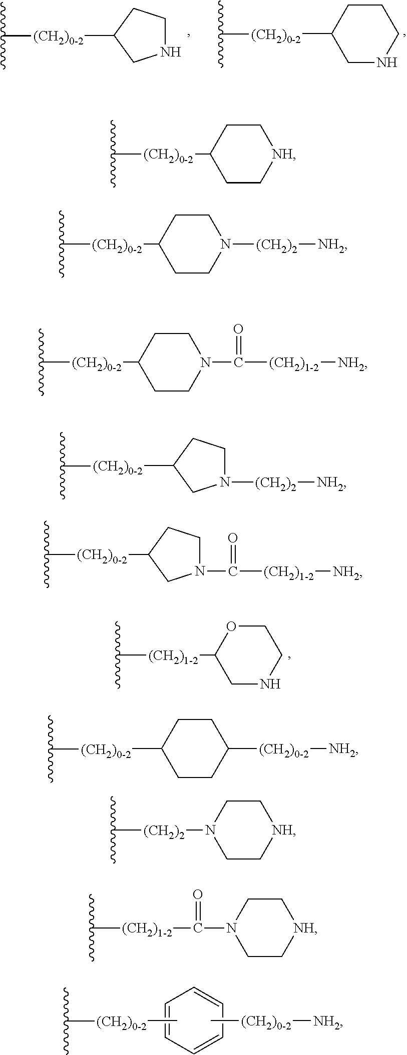

### and —(CH₂)₃₋₇—NH₂.

In another embodiment, R² is chosen from

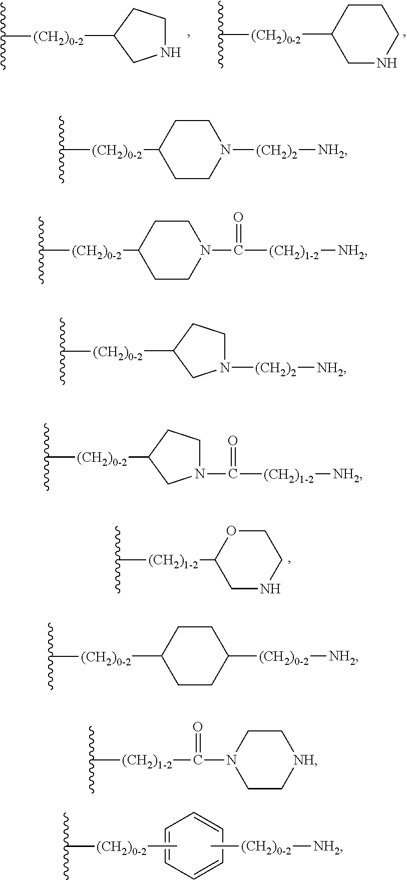

### and —(CH₂)₃₋₇—NH₂.

In another embodiment, R³ is

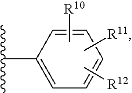

wherein
R¹⁰, R¹¹ and R¹² are independently chosen from —H, halogen, —OCH₃, —CF₃, —OCF₃, —CN, C₁-C₄ alkyl, —NR¹³R¹⁴, —S(O)ₘCH₃, —CONHR²², —NHCOR²³, —OR²⁴ and —NHS(O)ₘR²⁵;
wherein
R¹³ and R¹⁴ are independently chosen from —H and C₁-C₄ alkyl;
R²², R²³ and R²⁴ are one or two substituents independently chosen from —H, C₁-C₄ alkyl, C₁-C₆ cycloalkyl, aryl, —(CH₂)ₙNR²⁶R²⁷ and —(CH₂)ₙOR²⁸ said C₁-C₄ alkyl and C₁-C₆ cycloalkyl being optionally substituted with one or more halogens;
R²⁵ is C₁-C₄ alkyl;
R²⁶ and R²⁷ are independently chosen from H and C₁-C₄ alkyl or
R²⁶ and R²⁷ with the N to which they are attached form a 4-7 membered saturated heterocyclic ring optionally comprising an O;
R²⁸ is chosen from H and C₁-C₄ alkyl;
m is 0, 1 or 2 and
n is 1, 2 or 3.

In another embodiment R³ is chosen from pyridyl, thienyl, thiazolyl and furanyl optionally substituted with methyl or halogens.

In another embodiment is a compound selected from:
(R)—N-(3-Chloro-4-fluorobenzyl)-8-(2,6-dichlorophenyl)-9-(piperidin-3-ylmethyl)-9H-purin-2-amine;
(R)-8-(2,6-Dichlorophenyl)-N-(3-Chloro-4-fluorobenzyl)-9-(piperidin-3-ylmethyl)-9H-purin-2-amine;

(R)—N-(3-Chloro-6-fluorobenzyl)-8-(2,6-dichlorophenyl)-9-(piperidin-3-ylmethyl)-9H-purin-2-amine;
(R)-8-(2,6-Dichlorophenyl)-N-(3-fluorobenzyl)-9-(piperidin-3-ylmethyl)-9H-purin-2-amine;
(R)-8-(2,6-Dichlorophenyl)-N-(2,5-difluorobenzyl)-9-(piperidin-3-ylmethyl)-9H-purin-2-amine;
(R)-8-(2,6-Dichlorophenyl)-N-(4-fluorobenzyl)-9-(piperidin-3-ylmethyl)-9H-purin-2-amine;
(R)—N-(3,4-Dichlorobenzyl)-8-(2,6-dichlorophenyl)-9-(piperidin-3-ylmethyl)-9H-purin-2-amine;
(R)—N-(3-Chloro-4-fluorobenzyl)-8-(2-chloro-6-fluorophenyl)-9-(piperidin-3-ylmethyl)-9H-purin-2-amine;
(R)-8-(2,6-Dichlorophenyl)-N-(2,4-difluorobenzyl)-9-(piperidin-3-ylmethyl)-9H-purin-2-amine;
(R)-8-(2,6-Dichlorophenyl)-N-(2-fluorobenzyl)-9-(piperidin-3-ylmethyl)-9H-purin-2-amine;
(S)-8-(2,6-Dichlorophenyl)-N-(2-fluorobenzyl)-9-(piperidin-3-ylmethyl)-9H-purin-2-amine;
(R)—N-benzyl-8-(2,6-dichlorophenyl)-9-(piperidin-3-ylmethyl)-9H-purin-2-amine;
(R)-8-(2-chloro-6-fluorophenyl)-N-(3,4-difluorobenzyl)-9-(piperidin-3-ylmethyl)-9H-purin-2-amine;
(R)-8-(2,6-Dichlorophenyl)-N-(thien-3-ylmethyl)-9-(piperidin-3-ylmethyl)-9H-purin-2-amine;
(R)—N-(3-Chloro-6-fluorobenzyl)-8-(2-chloro-6-fluorophenyl)-9-(piperidin-3-ylmethyl)-9H-purin-2-amine;
(R)-8-(2,6-Dichlorophenyl)-N-(2-fluoro-1-ethylphenyl)-9-(piperidin-3-ylmethyl)-9H-purin-2-amine;
(R)-8-(2,6-Dichlorophenyl)-N-(2-fluorobenzyl)-9-(piperidin-3-ylmethyl)-9H-purin-2-amine;
(R)-8-(2,6-Dichlorophenyl)-N-(3,4-difluorobenzyl)-9-(piperidin-3-ylmethyl)-9H-purin-2-amine;
(R)-4-(8-(2,6-Dichlorophenyl)-9-(piperidin-3-ylmethyl)-9H-purin-2-yl-amino)methylphenol;
(R)-3-(8-(2,6-Dichlorophenyl)-9-(piperidin-3-ylmethyl)-9H-purin-2-yl-amino)methylphenol;
(R)-2-fluoro-4-(8-(2,6-Dichlorophenyl)-9-(piperidin-3-ylmethyl)-9H-purin-2yl-amino)methylphenol;
(R)-8-(2,6-Dichloro-4-hydroxymethylphenyl)-N-(3,4-difluorobenzyl)-9-(piperidin-3-ylmethyl)-9H-purin-2-amine;
(R)-8-(2,4-Dichloro-6-trifluoromethoxyphenyl)-N-(3,4-difluorobenzyl)-9-(piperidin-3-ylmethyl)-9H-purin-2-amine;
(R)-8-(2,6-Dichloro-4-ethoxyphenyl)-N-(3,4-difluorobenzyl)-9-(piperidin-3-ylmethyl)-9H-purin-2-amine;
(R)-8-(2,6-Dichloro-4-methylphenyl)-N-(3,4-difluorobenzyl)-9-(piperidin-3-ylmethyl)-9H-purin-2-amine;
(R)-3,5-dichloro-4-(2-(3,4-difluorobenzyl)amino-9-(piperidin-3-ylmethyl)-9H-purin-8-yl)phenol;
(R)-8-(2,6-Dichloro-4-fluorophenyl)-N-(3,4-difluorobenzyl)-9-(piperidin-3-ylmethyl)-9H-purin-2-amine;
(R)-8-(4-Bromo-2,6-dichlorophenyl)-N-(3,4-difluorobenzyl)-9-(piperidin-3-ylmethyl)-9H-purin-2-amine;
(R)-8-(4-Amino-2,6-dichlorophenyl)-N-(3,4-difluorobenzyl)-9-(piperidin-3-ylmethyl)-9H-purin-2-amine;
(R)-8-(4-Cyclopropylmethyl-2,6-dichlorophenyl)-N-(3,4-difluorobenzyl)-9-(piperidin-3-ylmethyl)-9H-purin-2-amine;
(R)-8-(2,6-Dichloro-4-(3-dimethylaminopropyl)phenyl)-N-(3,4-difluorobenzyl)-9-(piperidin-3-ylmethyl)-9H-purin-2-amine;
(R)-8-(2,6-Dichloro-4-(2-dimethylaminoethyl)phenyl)-N-(3,4-difluorobenzyl)-9-(piperidin-3-ylmethyl)-9H-purin-2-amine;
(R)-8-(2,6-Dichloro-4-(2-hydroxyethyl)phenyl)-N-(3,4-difluorobenzyl)-9-(piperidin-3-ylmethyl)-9H-purin-2-amine;
(R)-8-(2,6-Dichloro-4-(3-hydroxy-2-methylpropyl)phenyl)-N-(3,4-difluorobenzyl)-9-(piperidin-3-ylmethyl)-9H-purin-2-amine;
(R)-8-(2,6-Dichloro-4-(2-methoxyethyl)phenyl)-N-(3,4-difluorobenzyl)-9-(piperidin-3-ylmethyl)-9H-purin-2-amine;
(R)-8-(2,6-Dichloro-4-(3-methoxypropyl)phenyl)-N-(3,4-difluorobenzyl)-9-(piperidin-3-ylmethyl)-9H-purin-2-amine;
(R)—N-(3,5-Dichloro-4-(2-(3,4-difluorobenzylamino)-9-(piperidin-3-ylmethyl)-9H-purin-8yl-phenyl acetamide and
(R)—N-(3,5-Dichloro-4-(2-(3,4-difluorobenzylamino)-9-(piperidin-3-ylmethyl)-9H-purin-8yl-phenyl-3,3,3-trifluoropropyl amide
   or a pharmaceutically acceptable salt thereof.

In one embodiment, the invention is directed to a method of treatment of a T-cell mediated disease comprising administering a therapeutically effective amount of a compound of formula I, or salt thereof. The T-cell mediated disease may be, for example, an autoimmune disease or an inflammatory disease. The autoimmune disease, may be, for example, rheumatoid arthritis or lupus erythematosus. The inflammatory disease may be, for example, asthma or inflammatory bowel disease.

In another embodiment, the invention is directed to a method of treatment of cancer, such as gastrointestinal cancer, comprising administering a therapeutically effective amount of a compound of formula I, or salt thereof.

In yet another embodiment, the invention is directed to a method of treatment of diabetes comprising administering a therapeutically effective amount of a compound of formula I, or salt thereof.

### DEFINITIONS

Throughout this specification the terms and substituents retain their definitions.

Alkyl and alkane, unless otherwise specified, are intended to include linear, branched, or cyclic hydrocarbon structures and combinations thereof. Lower alkyl refers to alkyl groups of from 1 to 6 carbon atoms. Examples of lower alkyl groups include methyl, ethyl, propyl, isopropyl, butyl, s- and t-butyl and the like. Preferred alkyl groups are those of C₂₀ or below. Cycloalkyl is a subset of alkyl and includes cyclic hydrocarbon groups of from 3 to 8 carbon atoms. Examples of cycloalkyl groups include c-propyl, c-butyl, c-pentyl, norbornyl and the like.

(C₁ to Cₙ) Hydrocarbon includes alkyl, cycloalkyl, alkenyl, alkynyl, aryl and combinations thereof containing only hydrogen and one to n carbons. Examples include vinyl, allyl, cyclopropyl, propargyl, phenethyl, cyclohexylmethyl, camphoryl and naphthylethyl.

Saturated (C₁ to Cₙ)hydrocarbon is identical in meaning to (C₁ to Cₙ)alkyl or (C₁ to Cₙ)alkane as used herein. Whenever reference is made to C₀₋ₙ alkyl, (C₀ to Cₙ)alkyl, or (C₀ to Cₙ)alkane when number of carbon atoms is 0, a direct bond is implied.

Alkoxy or alkoxyl refers to groups of from 1 to 8 carbon atoms of a straight, branched, cyclic configuration and combinations thereof attached to the parent structure through an oxygen. Examples include methoxy, ethoxy, propoxy, isopropoxy, cyclopropyloxy, cyclohexyloxy and the like. Lower-alkoxy refers to groups containing one to four carbons.

Fluoroalkyl refers to alkyl residues in which one or more hydrogens have been replaced by fluorine. It includes perfluoroalkyl, in which all the hydrogens have been replaced by fluorine. Examples include fluoromethyl, difluoromethyl, trifluoromethyl, trifluoroethyl and pentafluoroethyl.

Oxaalkyl refers to alkyl residues in which one or more carbons (and their associated hydrogens) have been replaced by oxygen. Examples include methoxypropoxy, 3,6,9-trioxadecyl and the like. The term oxaalkyl is intended as it is understood in the art [see Naming and Indexing of Chemical Substances for Chemical Abstracts, published by the American Chemical Society, ¶196, but without the restriction of ¶127(a)], i.e. it refers to compounds in which the oxygen is bonded via a single bond to its adjacent atoms (forming ether bonds); it does not refer to doubly bonded oxygen, as would be found in carbonyl groups. Similarly, thiaalkyl and azaalkyl refer to alkyl residues in which one or more carbons has been replaced by sulfur or nitrogen, respectively. Examples include ethylaminoethyl and methylthiopropyl.

Acyl refers to groups of from 1 to 8 carbon atoms of a straight, branched, cyclic configuration, saturated, unsaturated and aromatic and combinations thereof, attached to the parent structure through a carbonyl functionality. One or more carbons in the acyl residue may be replaced by nitrogen, oxygen or sulfur as long as the point of attachment to the parent remains at the carbonyl. Examples include acetyl, benzoyl, propionyl, isobutyryl, t-butoxycarbonyl, benzyloxycarbonyl and the like. Lower-acyl refers to groups containing one to four carbons.

Cyclyl refers to a 3- to 8-membered ring containing 0-3 heteroatoms selected from O, N, or S; a bicyclic 9- or 10-membered ring system containing 0-3 heteroatoms selected from O, N, or S; or a tricyclic 13- or 15-membered ring system containing 0-3 heteroatoms selected from O, N, or S. Cyclyl may be saturated, unsaturated, or aromatic. A carbocyclyl is a cyclyl lacking any heteroatoms. As commonly understood, when referring to cyclyl as a substituent, it is intended that the point of attachment is a ring carbon or heteroatom of the cyclyl group.

Cyclylalkyl refers to an alkyl residue attached to a cyclyl. As commonly understood, when referring to cyclylalkyl as a substituent, it is intended that the point of attachment is the alkyl group.

Cycloalkylalkyl refers to an alkyl residue attached to a cycloalkyl. As commonly understood, when referring to cycloalkylalkyl as a substituent, it is intended that the point of attachment is the alkyl group.

Alicyclyl refers to aliphatic compounds having a carbocyclic ring structure which may be saturated or unsaturated, but may not be a benzenoid or other aromatic system. Alicyclyl may be a 3- to 8-membered ring containing 0-3 heteroatoms selected from O, N, or S; a bicyclic 9- or 10-membered ring system containing 0-3 heteroatoms selected from O, N, or S; or a tricyclic 13- or 15-membered ring system containing 0-3 heteroatoms selected from O, N, or S. A carboalicyclyl is an alicyclyl lacking any heteroatoms. As commonly understood, when referring to alicyclyl as a substituent, it is intended that the point of attachment is a ring carbon or heteroatom of the alicyclyl group.

Alicyclylalkyl refers to an alkyl residue attached to an alicyclyl. As commonly understood, when referring to alicyclylalkyl as a substituent, it is intended that the point of attachment is the alkyl group.

Aryl and heteroaryl mean a 5- or 6-membered aromatic or heteroaromatic ring containing 0-3 heteroatoms selected from O, N, or S; a bicyclic 9- or 10-membered aromatic or heteroaromatic ring system containing 0-3 heteroatoms selected from O, N, or S; or a tricyclic 13- or 14-membered aromatic or heteroaromatic ring system containing 0-3 heteroatoms selected from O, N, or S. As commonly understood, when referring to aryl as a substituent, it is intended that the point of attachment is a ring carbon of the aryl group (or ring carbon or heteroatom of the heteroaryl). For the purpose of the present invention, aryl and heteroaryl refer to systems in which at least one ring, but not necessarily all rings, are fully aromatic. Thus aromatic 6- to 14-membered carbocyclic rings include, e.g., benzene, naphthalene, indane, tetralin, benzocycloheptane and fluorene and the 5- to 10-membered aromatic heterocyclic rings include, e.g., imidazole, pyridine, indole, isoindoline, thiophene, benzopyranone, thiazole, furan, benzimidazole, quinoline, isoquinoline, tetrahydroisoquinoline, quinoxaline, tetrahydrocarboline, pyrimidine, pyrazine, tetrazole and pyrazole.

Arylalkyl means an alkyl residue attached to an aryl ring. As commonly understood, when referring to arylalkyl as a substituent, it is intended that the point of attachment is the alkyl group. Examples of arylalkyl are benzyl, phenethyl, phenylpropyl and naphthylethyl. Heteroarylalkyl means an alkyl residue attached to a heteroaryl ring. Examples include, e.g., pyridinylmethyl, pyrimidinylethyl and the like.

Heterocycle means a cycloalkyl or aryl residue in which from one to three carbons is replaced by a heteroatom selected from the group consisting of N, O and S. The nitrogen and sulfur heteroatoms may optionally be oxidized, and the nitrogen heteroatom may optionally be quaternized. Heterocycles also include spiroheterocycles. It is to be noted that heteroaryl is a subset of heterocycle in which the heterocycle is aromatic. Examples of heterocyclyl residues additionally include piperazinyl, 4-piperidinyl, pyrazolidinyl, imidazolyl, imidazolinyl, imidazolidinyl, pyrazinyl, oxazolidinyl, isoxazolidinyl, thiazolidinyl, isothiazolyl, quinuclidinyl, isothiazolidinyl, benzimidazolyl, thiadiazolyl, benzopyranyl, benzothiazolyl, tetrahydrofuryl, tetrahydropyranyl, thienyl, benzothienyl, thiamorpholinyl, thiamorpholinylsulfoxide, thiamorpholinylsulfone, oxadiazolyl, triazolyl and tetrahydroquinolinyl.

Whenever reference is made to nitrogen attached cyclyl or nitrogenous cyclyl (where cyclyl may be identified as heterocyclyl, alicyclyl, or heteroaryl) such cyclyl contains at least one N atom, but may also contain additional 0-3 heteroatoms selected from O, N, or S.

Aminoalkyl means an amino group bound to a core structure via an alkyl group, e.g., aminomethyl, aminoethyl, aminopenthyl, etc. The alkyl group, as defined above, could be straight or branched and, therefore, an aminoalkyl includes, e.g., —CH₂CH₂CH(CH₃)CH₂NH₂, —CH₂C(CH₃)₂CH₂NH₂, etc. Alkylaminoalkyl means a secondary amine bound to a core structure via an alkyl group, e.g., —CH₂CH₂NHCH₃, —CH₂CH₂CH₂NHCH₂CH₃, etc. Dialkylaminoalkyl means a tertiary amine bound to a core structure via an alkyl group, e.g., —CH₂N(CH₃)₂, —CH₂CH₂CH₂N(CH₃)CH₂CH₃, etc.

Substituted alkyl, cyclyl, aryl, cycloalkyl, heterocyclyl etc. refer to alkyl, cyclyl, aryl, cycloalkyl, or heterocyclyl wherein up to three H atoms in each residue are replaced with loweralkyl, halogen, haloalkyl, hydroxy, hydroxymethyl, loweralkoxy, perfluoroloweralkoxy, carboxy, carboalkoxy (also referred to as alkoxycarbonyl), carboxamido (also referred to as alkylaminocarbonyl), sulfonamido, aminosulfonyl, alkylaminosulfonyl, cyano, carbonyl, nitro, amino, alkylamino, dialkylamino, ureido, allylureido, mercapto, alkylthio, sulfoxide, sulfone, acylamino, amidino, alkylthio, alkylsulfinyl, alkylsulfonyl, phenyl, benzyl, heteroaryl, phenoxy, benzyloxy, or heteroaryloxy.

The term “halogen” means fluorine, chlorine, bromine or iodine.

As used herein, reference to “treatment” or “treating” a patient are intended to include prophylaxis. The terms include amelioration, prevention and relief from the symptoms and/or effects associated with these disorders. The terms “preventing” or “prevention” refer to administering a medicament beforehand to forestall or obtund an attack. Persons of ordinary skill in the medical art (to which the present method claims are directed) recognize that the term “prevent” is not an absolute term. In the medical art it is understood to refer to the prophylactic administration of a drug to diminish the likelihood or seriousness of a condition, and this is the sense intended.

### Abbreviations

The following abbreviations and terms have the indicated meanings throughout:
Ac=acetyl
anh.=anhydrous
ACN=acetonitrile
BNB=4-bromomethyl-3-nitrobenzoic acid
Boc=t-butyloxy carbonyl
Bu=butyl
CBZ=carbobenzoxy=benzyloxycarbonyl
CDI=carbonyl diimidazole
DBU=diazabicyclo[5.4.0]undec-7-ene
DCM=dichloromethane=methylene chloride=CH₂Cl₂
DEAD=diethyl azodicarboxylate
DIC=diisopropylcarbodiimide
DIEA=N,N-diisopropylethyl amine
DMAP=4-N,N-dimethylaminopyridine
DMF=N,N-dimethylformamide
DMSO=dimethyl sulfoxide
DVB=1,4-divinylbenzene
EEDQ=2-ethoxy-1-ethoxycarbonyl-1,2-dihydroquinoline
Et=ethyl
FCC=flash column chromography
Fmoc=9-fluorenylmethoxycarbonyl
GC=gas chromatography
h=hour(s)
HATU=O-(7-Azabenzotriazol-1-yl)-1,1,3,3-tetramethyluronium hexafluorophosphate
HOAc=acetic acid
HOBt=hydroxybenzotriazole
Me=methyl
mesyl=methanesulfonyl
MTBE=methyl t-butyl ether
NMO=N-methylmorpholine oxide
PEG=polyethylene glycol
Ph or κ=phenyl
PhOH=phenol
PfP=pentafluorophenol
PPTS=pyridinium p-toluenesulfonate
PyBroP=bromo-tris-pyrrolidino-phosphonium hexafluorophosphate
rm=reaction mixture
rt=room temperature sat'd=saturated
TBDMS=t-butyldimethylsilyl
TFA=trifluoroacetic acid
THF=tetrahydrofuran
TIPSO=triisopropylsilanyloxy
TMOF=trimethyl orthoformate
TMS=trimethylsilyl
TBDMS=t-butyldimethylsilyl
tosyl=p-toluenesulfonyl
Trt=triphenylmethyl

Although this invention is susceptible to embodiment in many different forms, preferred embodiments of the invention are shown. It should be understood, however, that the present disclosure is to be considered as an exemplification of the principles of this invention and is not intended to limit the invention to the embodiments illustrated.

It may be found upon examination that certain members of the claimed genus are not patentable to the inventors in this application. In this event, subsequent exclusions of species from the compass of applicants' claims are to be considered artifacts of patent prosecution and not reflective of the inventors' concept or description of their invention; the invention encompasses all of the members of the genus (I) that are not already in the possession of the public.

In general, the compounds of the present invention may be prepared by the methods illustrated in the general reaction schemes as, for example, described below, or by modifications thereof, using readily available starting materials, reagents and conventional synthesis procedures. In these reactions, it is also possible to make use of variants that are in themselves known, but are not mentioned here.

### General Synthesis of Purines

One method for preparing purine analogs of the invention is shown in Scheme 1. Displacement of the two chlorides in 2,4-dichloro-5-nitropyrimidine 1 usually occurs in a regioselective manner. Thus, the more reactive chloride in the 2-position is first displaced by an amine R′NH₂ to yield compound 2. Addition of a second amine R″NH₂ displaces the chloride in the 4-position. Reduction of the nitro group in 3 to an amine using reagents well known in the art (e.g. Raney Ni/H₂, Fe/EtOH/aqAcOH, Na₂S₂O₄/NR₄OH/H₂O/Dioxane), followed by cyclization with an aryl aldehyde gives purine 5.

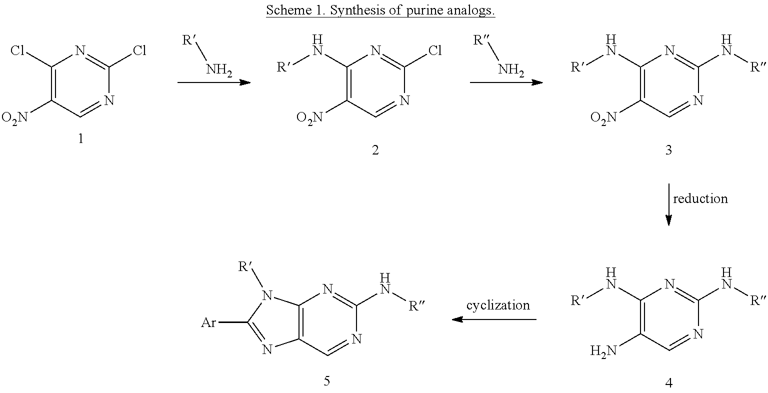

The purine analogs of the invention may be prepared on solid support (Scheme 2). For example, an acid cleavable linker can be attached to the Argogel-NH₂ resin. The resin with the linker is first reductive aminated with a R′NH₂. The pyrimidine 2, which is similarly prepared from the first step in Scheme 1, is then attached to the amine by a nucleophilic displacement reaction. Reduction of the nitro group, followed by ring closure with an aldehyde, yields the purine. The product can then be released from the solid support by treatment with acid such as trifloroacetic acid.

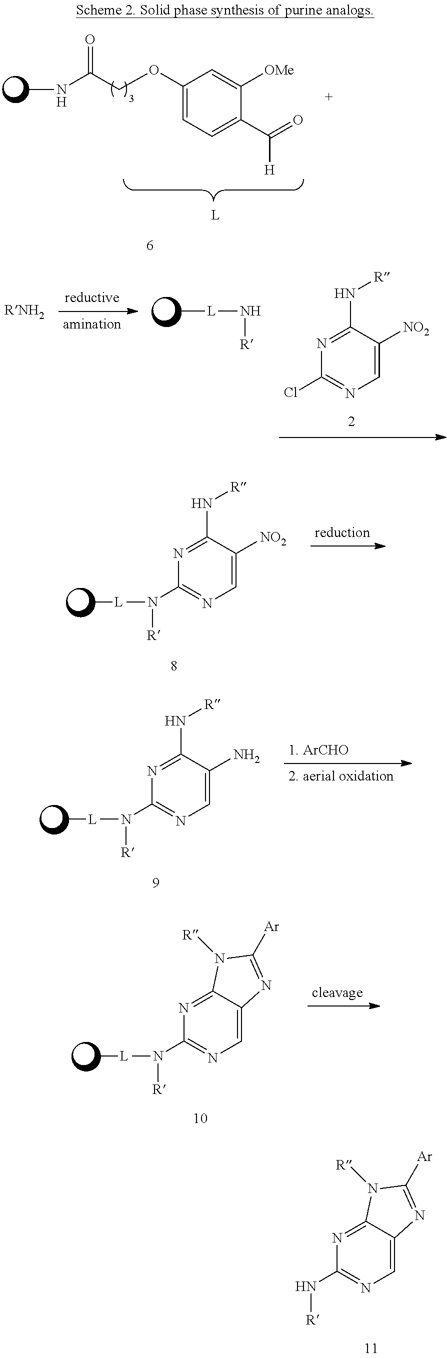

Following are exemplary procedures for preparation of some of the compounds of the invention.

### Synthesis of N-(3,4-difluorobenzyl)-8-(2-chloro-6-fluorophenyl)-9-((R)-piperidin-3-ylmethyl)-9H-purin-2-amine (Compound 113)

One possible process for synthesis of N-(3,4-difluorobenzyl)-8-(2-chloro-6-fluorophenyl)-9-((R)-piperidin-3-ylmethyl)-9H-purin-2-amine (Compound 113) is shown in Scheme 3 below and detailed in the following description.

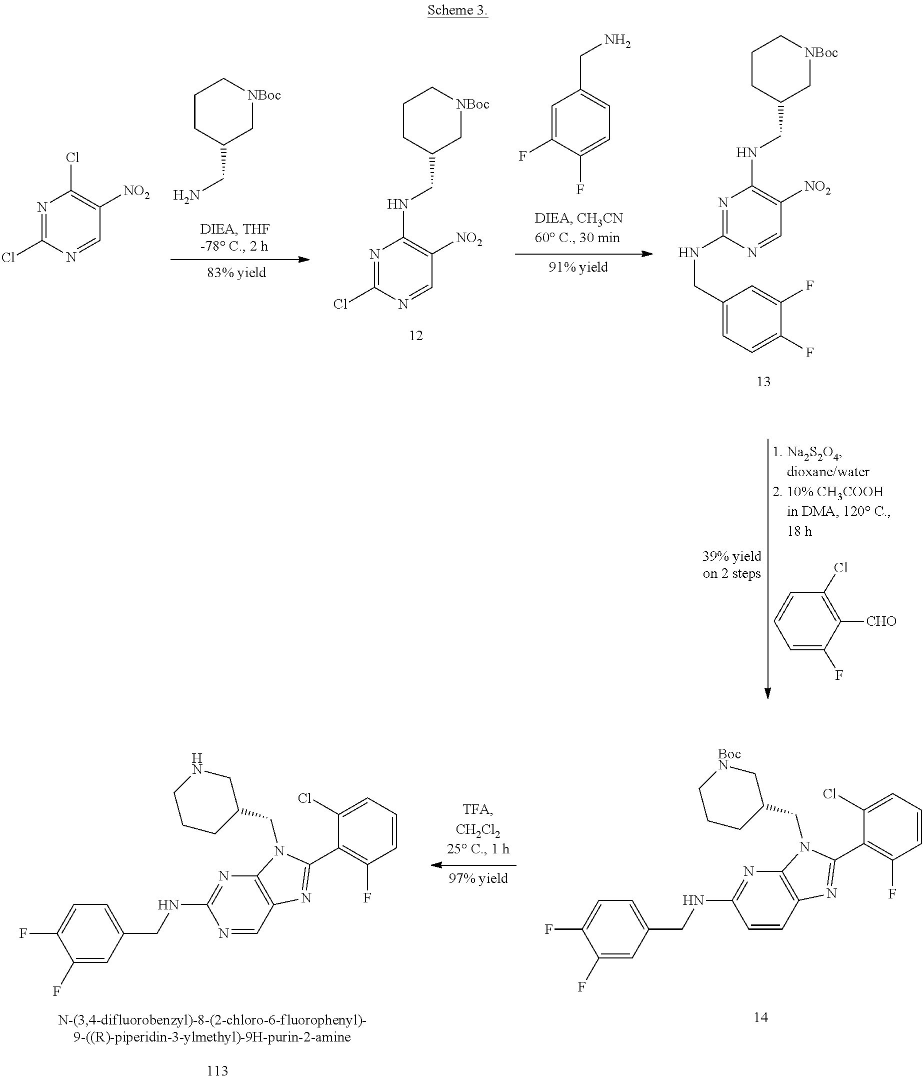

### (S)-tert-butyl 3-((2-chloro-5-nitropyrimidin-4-ylamino)methyl)piperidine-1-carboxylate (12)

To 1.267 g (6.53 mmol, 1.0 equiv.) of 2,4-dichloro-5-nitropyrimidine (Toronto Research Chemicals) in 8 mL of anhydrous THF at −78° C. was added dropwise a solution of 6.53 mmol (1 equiv.) of an amine and 1.25 mL of N,N-diisopropylethylamine in 6.5 mL anhydrous THF.

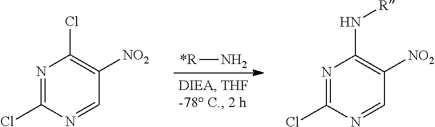

The reaction mixture was stirred for 30 min at −78° C. and then allowed to warm to 25° C. and stirred for an additional 1 h. The solvent was removed in vacuo and the residue purified by flash chromatography on silica gel.

(S)-tert-butyl 3-((2-chloro-5-nitropyrimidin-4-ylamino)methyl)piperidine-1-carboxylate (12):

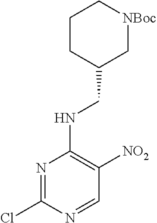

was synthesized using the procedure described above, using (S)-1-Boc-3-(aminomethyl)piperidine (1.4 g, 6.53 mmol, CNH Technologies) as the amine. Purification was performed on silica gel, using a 6/1 mixture of hexanes/ethyl acetate as the mobile phase. The desired product was obtained as a yellow solid (1.90 g) in 78% yield. NMR (300 MHz, CDCl₃), ppm: 9.05 (s, 1H), 8.56 (br s, 1H), 3.85 (dd, 2H), 3.59 (m, 2H), 3.03 (br t, 1H), 2.87 (dd, 1H), 1.86 (m, 2H), 1.69 (m, 1H), 1.46 (s, 9H), 1.40 (m, 2H, overlapping with 1.46 ppm).

(S)-tert-butyl-3-((2-(3,4-difluorobenzylamino)-5-nitropyrimidin-4-ylamino)methyl)piperidine-1-carboxylate (13)

To a solution of (S)-tert-butyl 3-((2-chloro-5-nitropyrimidin-4-ylamino)methyl)piperidine-1-carboxylate 12 (0.161 g, 0.43 mmol, 1 equiv.) in 2 mL of acetonitrile was added N,N-diisopropylethylamine (0.064 g, 0.087 mL, 0.5 mmol, 1.15 equiv.) and 3,4-difluorobenzyl amine (0.068 g, 0.48 mmol, 1.1 equiv.) and the reaction mixture was heated with stirring at 60° C. for 30 min. The solvent was removed in vacuo and the residue was taken in ethyl acetate (30 mL), washed with water (2×10 mL) and brine (1×10 mL). The organic layer was dried (anhydrous Na₂SO₄) and concentrated in vacuo. The pale yellow residue was purified by column chromatography (silica gel, hexane/ethyl acetate 3/1) to give 0.188 g of desired product 13 (91% yield). ¹H NMR (300 MHz, CDCl₃), ppm: 8.80 (s, 1H), 8.60 (br t, 1H), 7.16 (m, 2H), 7.06 (m, 1H), 6.87 (br t, 1H), 4.58 (d, 2H), 3.80 (m, 2H), 3.43 (m, 2H), 2.88 (br, 1H), 2.55 (br, 1H), 1.78 (m, 2H), 1.64 (m, 1H), 1.43 (s, 9H), 1.44 (m, 1H, overlapping with 1.43 ppm), 1.22 (m, 1H); MS (EI) m/z 478.8 (MH)⁺.

### (3S)-tert-butyl-3-((2-(3,4-difluorobenzylamino)-8-(2-chloro-6-fluorophenyl)-9H-purin-9-yl)methyl)piperidine-1-carboxylate (14)

To a solution of 0.522 g (3.0 mmol, 12.5 equiv.) of sodium hydrosulfite in 4 mL of water and 0.2 mL of a saturated aqueous solution of ammonia was added a solution of (S)-tert-butyl 342-(3,4-difluorobenzylamino)-5-nitropyrimidin-4-ylamino)methyl)piperidine-1-carboxylate 13 (0.115 g, 0.24 mmol, 1 equiv.) in 2 mL of 1,4-dioxane. This solution was stirred for 30 min at 25° C., when TLC analysis showed no starting material was left. Ethyl acetate (100 mL) was added and the organic layer washed with water (3×30 mL) and brine (1×30 mL), dried (anhydrous Na₂SO₄) and concentrated in vacuo to give crude (S)-tert-butyl 3-((2-(3,4-difluorobenzylamino)-5-aminopyrimidin-4-ylamino)methyl)piperidine-1-carboxylate.

To a solution of crude (S)-tert-butyl 3-((2-(3,4-difluorobenzylamino)-5-aminopyrimidin-4-ylamino)methyl)piperidine-1-carboxylate in 2 mL of anhydrous N,N-dimethylacetamide and 0.2 mL of acetic acid in a 20 mL scintillation vial was added 2-chloro-6-fluorobenzaldehyde (0.076 g, 0.48 mmol, 2 equiv.). The reaction mixture was heated at 120° C. for 21 h, then allowed to cool to 25° C. The solution was diluted with ethyl acetate (60 mL), the organic layer was washed with water (2×20 mL) and brine (1×20 mL), dried (anhydrous Na₂SO₄) and concentrated in vacuo. The residue was purified by column chromatography (silica gel, hexane/ethyl acetate 3/2) to give the desired product 14 (0.056 g, 39% yield over 2 steps). ¹H NMR (300 MHz, CDCl₃), ppm: 8.75 (s, 1H), 7.54-7.46 (m, 1H), 7.39-7.36 (m, 1H), 7.23-7.06 (m, 4H), 5.74 (br t, 1H), 4.65 (d, 2H), 3.84-3.74 (m, 4H), 2.72 (m, 1H), 2.45 (ddd, 1H), 1.86 (br, 1H), 1.51-1.23 (m, 3H, overlapping with 1.36 ppm), 1.36 (s, 9H), 0.91 (m, 1H); MS (EI) m/z 587.0 (MH)⁺.

### N-(3,4-difluorobenzyl)-8-(2-chloro-6-fluorophenyl)-9-((R)-piperidin-3-ylmethyl)-9H-purin-2-amine (113)

To a solution of 0.0214 g (0.036 mmol) of (3S)-tert-butyl 3-((2-(3,4-difluorobenzylamino)-8-(2-chloro-6-fluorophenyl)-9H-purin-9-yl)methyl)piperidine-1-carboxylate 14 in 0.5 mL methylene chloride was added TFA (0.5 mL) with stirring at room temperature for 1 h. The solvent was removed in vacuo and the residue purified using preparative HPLC to give 0.0212 g (97% yield) of desired product 113 (TFA salt) as a colorless oil. ¹H NMR (300 MHz, CD₃OD), ppm: 8.82 (s, 1H), 7.69-7.61 (m, 1H), 7.48-7.45 (m, 1H), 7.35-7.27 (m, 2H), 7.19-7.14 (m, 2H), 4.72-4.58 (m, 2H), 4.05-3.79 (m, 2H), 3.26-3.09 (m, 4H), 2.72-2.52 (m, 2H), 2.13 (br, 1H), 1.86-1.73 (m, 1H), 1.69 (m, 2H), 1.01 (m, 1H)); MS (EI) m/z 487.2 (MH)⁺.

### Solid Phase Synthesis of Purines

One possible process for solid phase synthesis of purine analogs of the invention is demonstrated in Scheme 4 below and detailed in the following description.

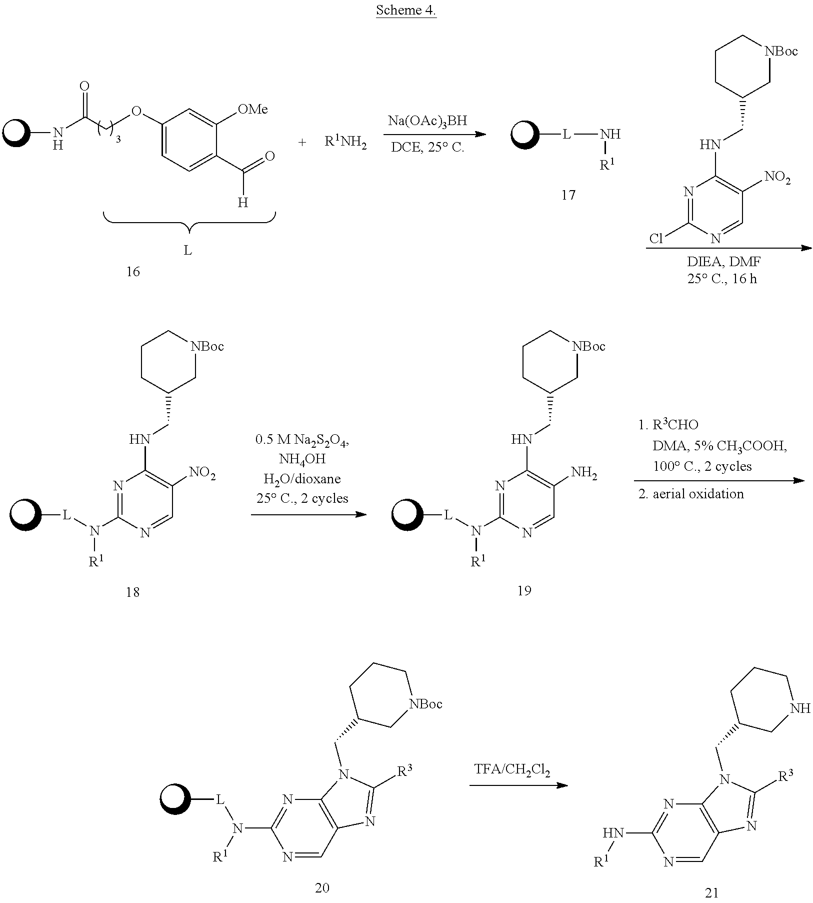

### Step 1: Reductive Amination with a Primary Amine

To a 100 mL shaking vessel containing a suspension of 1.2 g (0.786 mmol/g, 0.943 mmol, 1 equiv.) of resin-bound o-methoxybenzaldehyde resin 16 in 10 mL of 1,2-dichloroethane (DCE) was added 7.54 mmol (0.4 M, 8.0 equiv.) of an amine. The resin suspension was shaken for 1 min and 1.6 g (7.54 mmol, 0.4 M, 8.0 equiv.) of sodium triacetoxyborohydride was added followed by 10 mL of 1,2-dichloroethane. The suspension was shaken for 16 h at 25° C. The shaking vessel was then drained, and the resin was washed with CH₃OH (1×), CH₂Cl₂ (2×), CH₃OH (1×), CH₂Cl₂ (2×), CH₃OH (1×), CH₃OH (1×30 min) and CH₂Cl₂ (2×). The resulting resin-bound secondary amine 17 gave a positive result with the bromophenol blue staining test. The resin was dried in vacuo.

### Step 2: N-arylation with a 4-amino-2-chloro-5-nitropyrimidine

To 1.2 g (0.786 mmol/g, 0.943 mmol, 1 equiv.) of resin-bound secondary amine 17 in 4 mL of DMF and 0.33 mL (0.244 g, 1.886 mmol, 2.0 equiv.) of N,N-diisopropylethylamine in a shaking vessel was added a solution of 1.886 mmol (0.7 g, 0.25 M, 2.0 equiv.) of (S)-tert-butyl 3-((2-chloro-5-nitropyrimidin-4-ylamino)methyl)piperidine-1-carboxylate in 3.54 mL of DMF. The mixture was shaken at 25° C. for 16 h. The shaking vessel was drained and the resin was washed with DMF (2×), CH₂Cl₂ (1×), DMF (1×), CH₂Cl₂ (2×), CH₃OH (2×) and CH₂Cl₂ (2×). The resulting resin-bound nitropyrimidine resin 18 gave a negative result with the bromophenol blue staining tests. The resin was dried in vacuo.

### Step 3: Reduction of the Nitro Group

To a solution of 5.22 g (30.0 mmol, 0.5 M, 45 equiv.) of sodium hydrosulfite in 40 mL of water was added 20 mL of 1,4-dioxane followed by 0.93 mL of a saturated aqueous solution of ammonia. This solution was added to a 100 mL shaking vessel containing 1.2 g (0.786 mmol/g. 0.943 mmol, 1 equiv.) of resin-bound 5-nitropyrimidine 18. The resin suspension was shaken for 2 h at 25° C. The shaking vessel was drained and the resin was washed with water:1,4-dioxane 2:1 (v/v) (1×). The shaking vessel was recharged with 60 mL of a freshly prepared 0.5 M solution of sodium hydrosulfite in 40 mL of water and 20 mL of dioxane and 0.93 mL of a saturated aqueous solution of ammonia that was prepared as described above. The suspension was shaken at 25° C. for 16 h. The shaking vessel was drained and the resin was washed with water:1,4-dioxane 2:1 (v/v) (2×), anhydrous CH₃OH (2×), anhydrous DMF (2×), CH₂Cl₂ (2×) and anhydrous THF (2×). The resulting resin-bound 5-aminopyrimidine 19 gave a positive result with the bromophenol blue staining test. The resin was dried in vacuo.

### Step 4: Purine Formation

To a 20 mL scintillation vial containing 200 mg (0.786 mmol/g resin, 0.157 mmol, 1.0 equiv.) of the resin-bound 5-aminopyrimidine 19 was added 2 mL of a solution of 10.8 mmol (0.9 M, 12.5 equiv.) of an aldehyde in 10.8 mL of anhydrous N,N-dimethylacetamide and 0.2 mL of acetic acid. The resin suspension was heated at 100° C. for 21 h, then allowed to cool to 25° C. The solution was removed via pipette and the resin was washed with anhydrous N,N-dimethylacetamide (2×). The vial was recharged with 2.0 mL of a solution of 10.8 mmol (0.9 M, 12.5 equiv.) of the same aldehyde in 10.8 mL of N,N-dimethylacetamide and 0.2 mL of acetic acid. The resin suspension was heated at 100° C. for 16 h, then allowed to cool to 25° C. and transferred to a small shaking vessel. The vessel was drained and the resin was washed with DMF (4×), CH₂Cl₂ (2×), CH₃OH (2×) and CH₂Cl₂ (2×). The resulting resin-bound purine 20 was dried in vacuo.

Typical acid cleavage conditions were employed by stirring the resin in 10 mL of a 1:1 mixture of CH₂Cl₂/TFA (v/v) for 1 hour at 25° C. The resin suspension was then transferred to a small shaking vessel. The vessel was drained and the resin washed with CH₂Cl₂ (3×). Preparative HPLC purification of the combined filtrate gave the desired purine 21 (TFA salt).

### (R)-8-(2,6-dichlorophenyl)-9-(piperidin-3-ylmethyl)-N-(thiophen-2-ylmethyl)-9H-purin-2-amine (119)

(R)-8-(2,6-dichlorophenyl)-9-(piperidin-3-ylmethyl)-N-(thiophen-2-ylmethyl)-9H-purin-2-amine (119):

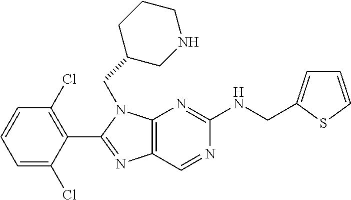

was prepared according to the above given procedure. 7.54 mmol (0.853 g, 0.4 M, 8.0 equiv.) of thiophene-2-methylamine were used in the step of reductive amination of resin with a primary amine. For the step of purine formation, the resin (1.2 g, 0.786 mmol/g, 0.943 mmol) was equally divided into 6 vials. In each vial, 2 mL of a solution of 10.8 mmol (0.9 M, 12.5 equiv.) of 2,6-dichloro benzaldehyde in 10.8 mL of anhydrous N,N-dimethylacetamide and 0.2 mL of acetic acid were added to 200 mg of resin-bound 5-aminopyrimidine (0.786 mmol/g, 0.157 mmol). Final preparative HPLC purification gave 160 mg of desired compound TFA salt as a colorless oil. The TFA salt was converted into the HCl salt by adding portions of 20 mL of a 1 M solution of HCl in ethanol (Alfa Aesar), stirring for 15 min at room temperature and in vacuo removing the solvent. The procedure was repeated 5 times. The sample was triturated with ether to give a light yellow solid, that recrystallized from methylene chloride/hexanes as a white solid (105 mg after being dried for 16 h over P₂O₅ under high vacuum at 40° C.). HCl salt: NMR (300 MHz, CD₃OD), ppm: 8.89 (s, 1H), 7.66 (m, 3H), 7.31 (m, 1H), 7.12 (br s, 1H), 6.97 (m, 1H), 4.92 (m, 2H), 3.98 (m, 2H), 3.85 (dd, 2H), 2.73 (m, 2H), 2.25 (br, 1H), 1.80 (m, 1H), 1.60 (m, 2H), 1.21 (m, 1H)); MS (EI) m/z 473.1 (M)⁺.

### Synthesis of (N)-(2-Chlorobenzyl)-8-ethyl-9-(2-(peperidin-4-yl)ethyl)-9H-purin-2-amine (477)

One possible process for solid phase synthesis of compound 477 is demonstrated in Scheme 5 below and detailed in the following description.

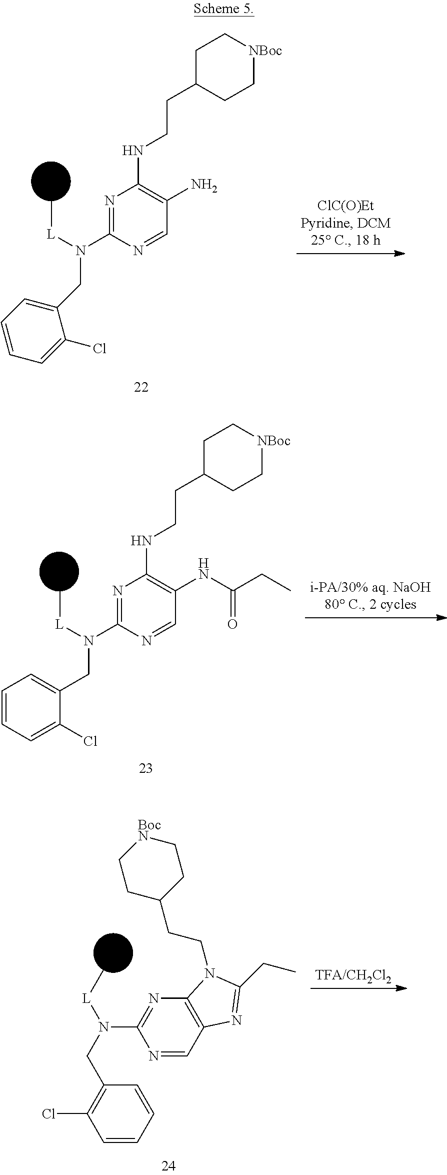

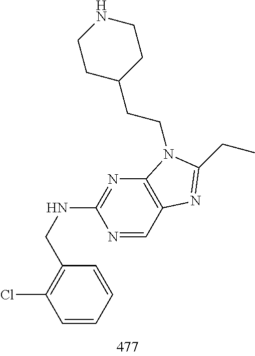

Intermediate 22 is similarly prepared by using the same solid phase method to prepare compound 19. Propionyl chloride (0.14 mL, 10 equiv.) was added to solid phase intermediate 22 (0.2 g, 0.8 mmol/g, 0.16 mmol) suspended in pyridine (2 mL) and DCM (1 mL) in a small shaker vessel that was shaken for 16 h at 25° C. The vessel was drained and the resins were washed with DCM (2×), MeOH (2×), DMF (1×), MeOH (2×) and DCM (2×). The resulting resin-bound amide gave a negative result with a bromophenol blue staining test.

The above amide was suspended in i-PA (1.5 mL) and transferred to a 20 mL scintillation vial. A 30% aq solution of NaOH (1 mL) was added and the mixture was slowly stirred at 80° C. for 16 hr and allowed to cool. The solution was removed via pipette and then recharged with i-PA (1.5 mL) and 30% aq NaOH (1 mL) and heated at 80° C. for 18 hr. The cooled mixture was transferred back to a small shaking vessel, drained and the resins were rinsed with i-PA/H₂O (2:1, 2×), MeOH (2×), DCM (1×), MeOH (2×) and DCM (2×).

The resulting resin-bound 8-ethyl purine derivative was cleaved from the resin following the typical acid cleavage procedure and purified via preparative RP-HPLC to yield the titled compound 477 (3.1 mg) as a TFA salt: ¹H NMR (300 MHz, CD₃OD), ppm: 8.64 (s, 1H), 7.47 (m, 2H), 7.30 (m, 2H), 4.81 (m, 2H), 4.20 (dd, 2H), 3.36 (m, 2H), 3.00 (q, 2H), 2.90 (m, 2H), 1.99 (d, 2H), 1.74 (q, 2H), 1.60 (m, 2H), 1.45 (t, 3H)); MS (EI) m/z 399.1/40.2 (M)⁺.

### Further Alternative Synthesis of Purines

1. One alternative process for preparing purine analogs of the invention is shown in Scheme 4. Variation of the R1-position on the purine scaffold could be accomplished by substitution of a SO₂Me-group at the R1-position. By using this route (Scheme 6), variation is introduced in a later stage of the synthesis compared with the route given in Scheme 1. As given in Scheme 1, displacement of the two chlorides in 2,4-dichloro-5-nitropyrimidine 1 usually occurs in a regioselective manner. Thus, the more reactive chloride in the 2-position is first displaced by an amine R′NH₂ to yield compound 2. Addition of NaSMe displaces the chloride in the 4-position. Reduction of the nitro group in 25 to an amine (26) using reagents well known in the art (e.g. Na₂S₂O₄/NH₄OH/H₂O/dioxane, Pd(C)/H₂/MeOH), followed by cyclization with an aryl aldehyde gives purine 27. Oxidation of the MeS-substituent to the corresponding sulfone and replacement of this leaving group with an amine gives the substituted purine 5.

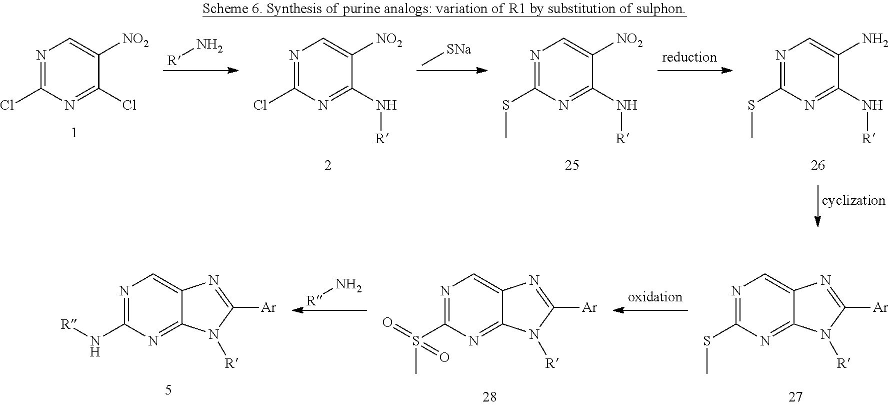

The following is an exemplary procedure for preparation of some of the compounds of the invention.

### Synthesis of (R)-4-((8-(2,6-dichlorophenyl)-9-(piperidin-3-ylmethyl)-9H-purin-2-yl-amino)methyl)-2-fluorophenol 2,2,2-trifluoroacetate (compound 506)

One possible process for synthesis of (R)-4-((8-(2,6-dichlorophenyl)-9-(piperidin-3-ylmethyl)-9H-purin-2-ylamino)methyl)-2-fluorophenol 2,2,2-trifluoroacetate (506) is shown in Scheme 7 below and detailed in the following description.

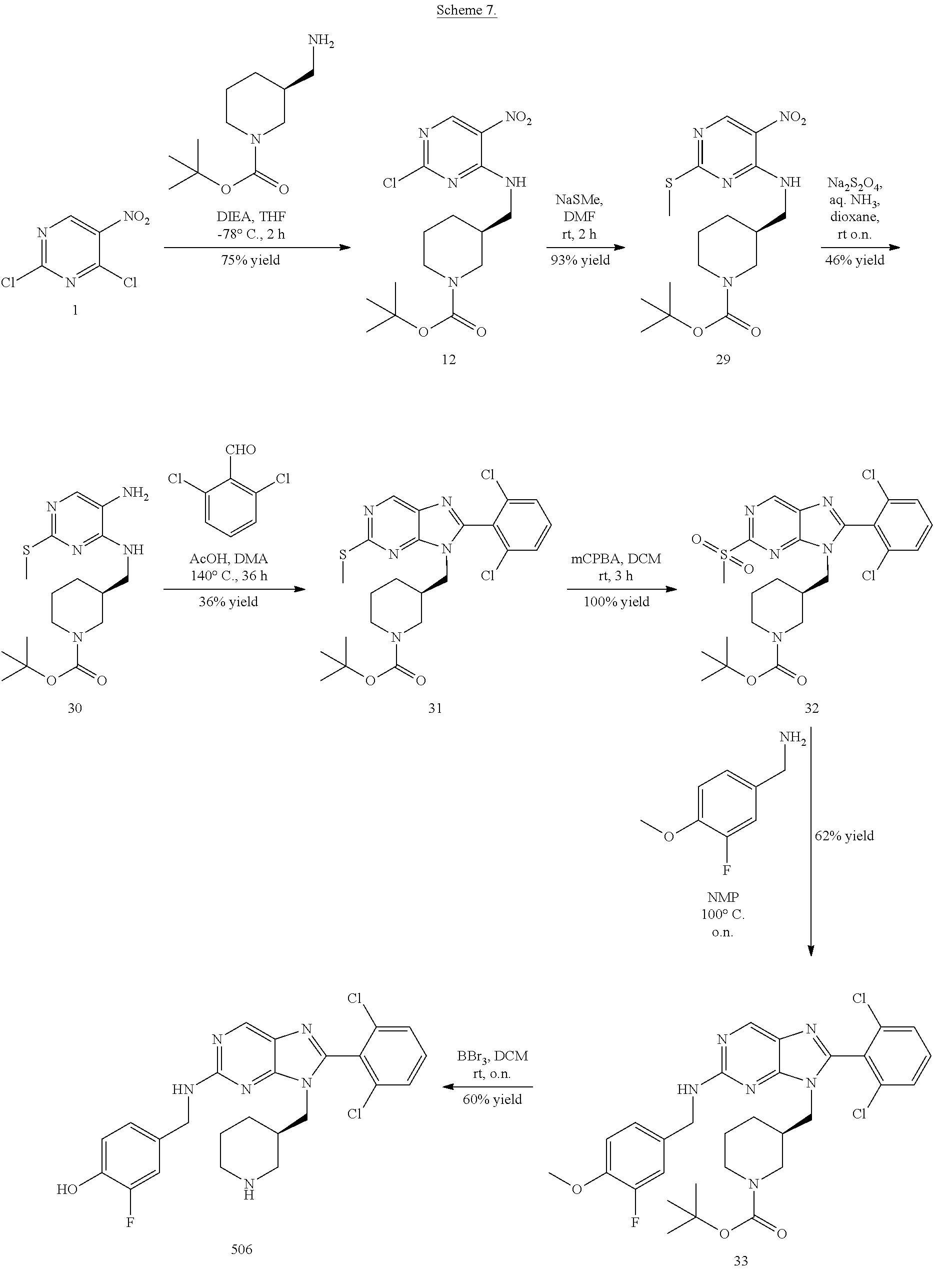

Synthesis of compound 12 has been described previously (Scheme 3).

### Compound 29

To a solution of compound 12 (24.85 g, 66.8 mmol, 1.0 equiv.) in DMF (70 ml), was added NaSMe (5.15 g, 73.5 mmol, 1.1 equiv.), resulting in a orange suspension. This was stirred at rt for 2 h. After observing complete conversion by NMR of the mixture, the mixture was diluted with EtOAc and washed with water (3×) followed by washing with brine. The organic layer was dried (Na₂SO₄), filtered and concentrated in vacuo. 23.93 g of solid compound 29 was obtained in 93% yield.

### Compound 30

To a solution of compound 29 (23.93 g, 62.4 mmol, 1.0 equiv.) in dioxane (100 ml), a saturated solution of Na₂S₂O₄ (50 g, 287 mmol, 4.6 equiv.) in water was added followed by aq. NH₃ (10 ml). Reaction mixture was stirred at rt o.n. The mixture was diluted with EtOAc and washed with water (4×) and brine. The organic layer was dried (Na₂SO₄), filtered and concentrated in vacuo. 10.25 g of compound 30 was obtained as a off-white solid in 46% yield.

### Compound 31

To a solution of compound 30 (10.25 g, 29 mmol, 1.0 equiv.) in DMA (100 ml) was added 2,6-dichlorobenzaldehyde (7.6 g, 43 mmol, 1.5 equiv.) followed by AcOH (10 ml). The mixture was heated at 140° C. while air was bubbled through for 36 h. The mixture was then diluted with EtOAc and washed with water (3×) followed by brine. The organic layer was dried (Na₂SO₄), filtered and concentrated in vacuo. The crude product was purified over silica, using a 1:1 EtOAc/heptane mixture as mobile phase. 5.39 g of yellow solid 31 was obtained in 36% yield.

### Compound 32

To a solution of compound 31 (4.3 g, 8.46 mmol, 1.0 equiv.) in DCM at 0° C., m-CPBA (70%, 4.4 g, 17 mmol, 2.0 equiv.) was added. The mixture was allowed to warm to rt slowly and was stirred for 3 h. Mixture was then diluted with DCM and washed with NaHCO₃ (2×), followed by water (2×) and brine. The organic layer was dried (Na₂SO₄), filtered and concentrated in vacuo. Compound 32 was obtained in near quantitative yield (4.56 g) as a colorless solid.

### Compound 33

To a solution of compound 32 (0.185 mmol, 100 mg, 1.0 equiv.) in NMP (2 ml) was added 3-fluoro-4-methoxybenzylamine (1.850 mmol, 287 mg, 10 equiv.). The reaction mixture was heated to 100° C. and stirred overnight. Mixture was then poured into H₂O and extracted with EtOAc. The combined organic layers were washed with water and brine, dried (Na₂SO₄), filtered and concentrated in vacuo. 70 mg of compound 33 was obtained in 62% yield.

### (R)-4-((8-(2,6-dichlorophenyl)-9-(piperidin-3-ylmethyl)-9H-purin-2-ylamino)methyl)-2-fluorophenol 2,2,2-trifluoroacetate (506)

To a solution of compound 33 (0.065 mmol, 40 mg, 1.0 equiv.) in DCM (2 ml) was added BBr₃ (0.182 mmol, 0.018 ml, 45.6 mg, 2.8 equiv.). The reaction mixture was stirred overnight at rt. For workup, the reaction mixture was cooled to 0° C. and quenched with MeOH. This solution was concentrated in vacuo and purified by semi-prepHPLC (0% to 80% ACN with TFA). After lyophilizing compound 506 was obtained as the TFA-salt in 60% yield (40 mg).

TFA-salt: NMR (400 MHz, CDCl₃), ppm: 10.40 (br s, 1H), 9.39 (br s, 1H), 8.67 (s, 1H), 7.53 (m, 3H), 7.31 (m, 1H), 7.29 (m, 1H), 7.13 (dd, 1H), 7.04 (m, 2H), 4.56 (dd, 2H), 3.84 (m, 1H), 3.73 (m, 1H), 3.40 (t, 1H), 3.25 (m, 1H), 2.73 (m, 1H), 2.64 (m, 1H), 2.42 (m, 1H), 2.04 (m, 1H), 1.57 (m, 1H), 1.26 (m, 1H), 0.89 (m, 1H).

2. Another possible route for preparing purine analogs of the invention is shown in Scheme 8. Introduction of substituted anilines on the R1-position of the purine scaffold could be accomplished by substituting a chloride under more vigorous conditions. Further synthesis to obtain the purine analogs of the invention can be achieved by following the reaction steps given in earlier routes (reduction and cyclization, see also Scheme 1 and 3).

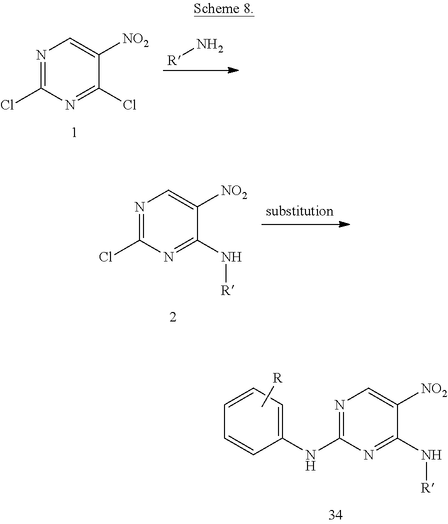

The following is an exemplary procedure for the preparation of a compound of the invention.

### Synthesis of (R)-8-(2,6-dichlorophenyl)-N-(3,4-dichlorophenyl)-9-(piperidin-3-ylmethyl)-9H-purin-2-amine 2,2,2-trifluoroacetate (compound 720)

One possible process for synthesis of (R)-8-(2,6-dichlorophenyl)-N-(3,4-dichlorophenyl)-9-(piperidin-3-ylmethyl)-9H-purin-2-amine 2,2,2-trifluoroacetate (720) is shown in Scheme 9 below and detailed in the following description.

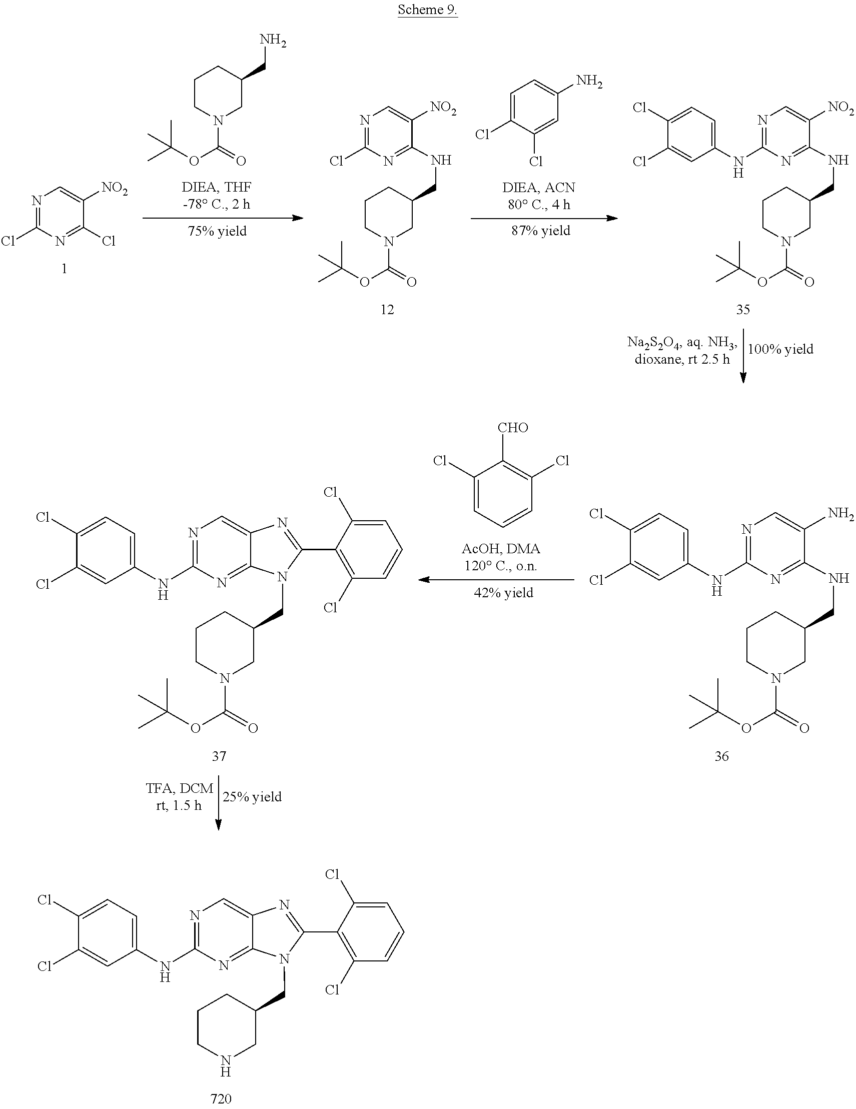

The synthesis of compound 12 is also described in Scheme 3 and detailed in the procedure below Scheme 3.

### Compound 35

To a solution of compound 12 (100 mg, 0.269 mmol, 1.0 equiv.) in ACN (3 ml), was added DIEA (54 μl, 0.309 mmol, 1.15 equiv.) and 3,4-dichloroaniline (55.7 mg, 0.344 mmol, 1.28 equiv.). The reaction mixture was stirred at 80° C. for 4 h. TLC showed that the reaction was complete. For workup, the rm was concentrated in vacuo, dissolved again in EtOAc, washed with water (twice) and brine. The crude product was concentrated again, purified over silica (eluens hept:EtOAc to 6:4). Product fractions were collected and concentrated in vacuo to obtain compound 35 in 87% yield.

### Compound 36

To a solution of Na₂S₂O₄ (512 mg, 2.94 mmol, 12.5 equiv.) in water (2 ml) and ammonia (aqueous sol., 189 μl, 4.23 mmol, 18.0 equiv.) was added a solution of compound 35 in dioxane (1 ml). The rm was stirred at rt for 2.5 h. After completion, EtOAc was added to the rm, followed by washing with water (3×) and brine. After drying on Na₂SO₄, filtration and concentration in vacuo, crude compound 36 was obtained in 100% yield.

### Compound 37

To a solution of compound 36 (110 mg, 0.235 mmol, 1.0 equiv.) in DMA (2 ml) was added 2,6-dichlorobenzaldehyde (82 mg, 0.471 mmol, 2.0 equiv.) and acetic acid (0.2 ml). The reaction mixture was stirred o.n. in a sealed tube at 120° C. After completion, the r.m. was cooled down to rt, diluted with EtOAc and washed with H₂O (3×) and brine. After drying on Na₂SO₄, filtration and concentration in vacuo, the crude product was purified by column chromatography (hept:EtOAc 9:1 to 1:1). Product fractions were collected and concentrated in vacuo to obtain compound 37 in 42% yield (62 mg).

### (R)-8-(2,6-dichlorophenyl)-N-(3,4-dichlorophenyl)-9-(piperidin-3-ylmethyl)-9H-purin-2-amine 2,2,2-trifluoroacetate (720)

To a solution of compound 37 (62 mg, 0.10 mmol) in DCM (1.5 ml) was added TFA (0.5 ml). The rm was stirred for 1.5 h at rt. The reaction mixture was concentrated in vacuo. After purification by prep-HPLC (0-70% ACN/TFA, 2×) and lyophilization, compound 720 was obtained as the TFA-salt in 25% yield (16 mg).

Compounds 513, 520, 522 and 530 (Scheme 10) can be synthesized using the synthetic route described in Scheme 1. The required aldehydes can be prepared according to literature procedures (Synthesis, 2004, no. 12, pp. 2062-2065).

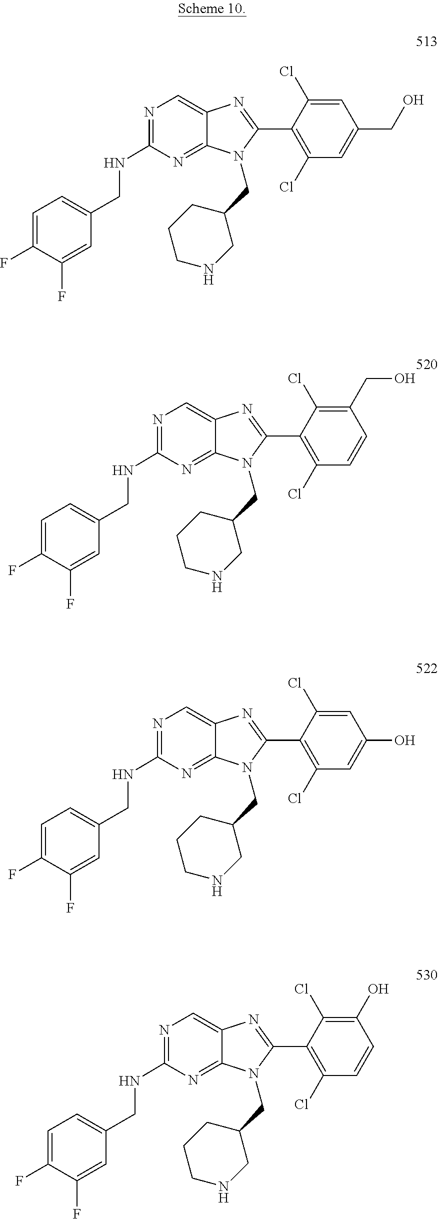

Compound 38 can be converted to ether derivatives (e.g. compound 519) as shown in Scheme 11.

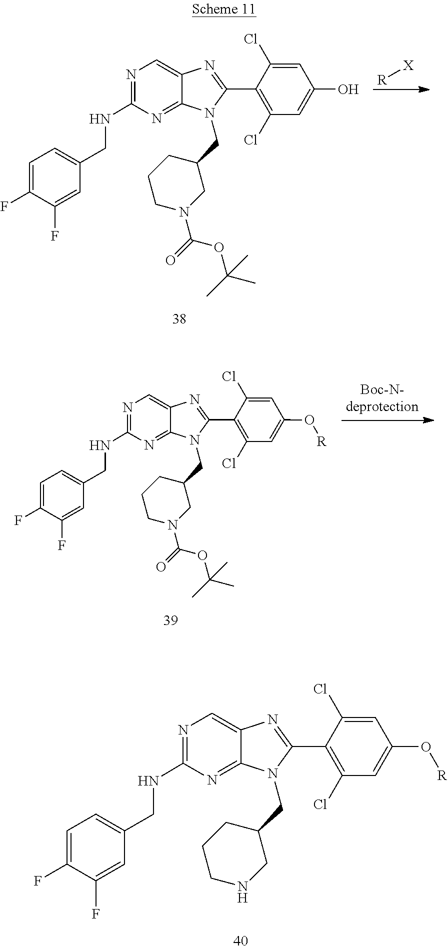

The following is an exemplary procedure for the preparation of a compound of the invention.

### Synthesis of (R)-8-(2,6-dichloro-4-ethoxyphenyl)-N-(3,4-difluorobenzyl)-9-(piperidin-3-ylmethyl)-9H-purin-2-amine 2,2,2-trifluoroacetate (compound 519)

One possible process for synthesis of (R)-8-(2,6-dichloro-4-ethoxyphenyl)-N-(3,4-difluorobenzyl)-9-(piperidin-3-ylmethyl)-9H-purin-2-amine 2,2,2-trifluoroacetate (519) is shown in Scheme 12 below and detailed in the following description.

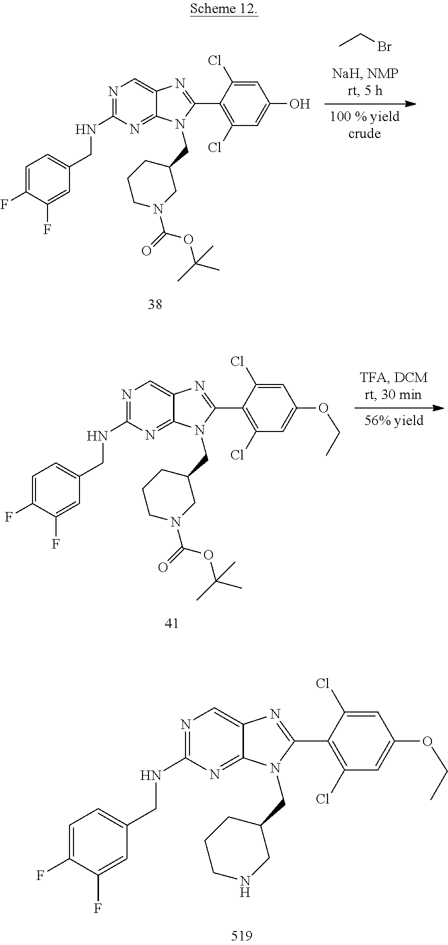

### Compound 41

To a solution of compound 38 (50 mg, 0.081 mmol, 1.0 equiv.) in NMP (1 ml) was added sodium hydride (9.68 mg, 0.242 mmol, 3.0 equiv.). The rm was stirred for 30 min at rt, then 1-bromoethane (0.030 ml, 0.404 mmol, 5.0 equiv.) was added. Rm was stirred at rt for 5 h to completion. For workup, the reaction mixture was poured out in water and extracted twice with EtOAc. Combined organic layers were washed with water (3×) and brine, dried on Na₂SO₄, filtered and concentrated in vacuo to obtain the crude compound 41 in a quantitative yield.

### (R)-8-(2,6-dichloro-4-ethoxyphenyl)-N-(3,4-difluorobenzyl)-9-(piperidin-3-ylmethyl)-9H-purin-2-amine 2,2,2-trifluoroacetate 519

To a solution of compound 41 (52 mg, 0.080 mmol) in DCM (1 ml) was added TFA (0.5 ml). Rm was stirred at it for 30 min. After completion, the rm was concentrated in vacuo, purified by prep-HPLC (0-50% ACN with TFA), concentrated and lyophilized to obtain the TFA-salt of compound 519 in a 56% yield over two steps.

TFA-salt: ¹H NMR (400 MHz, DMSO-D6), ppm: 8.74 (s, 1H), 8.58 (d, 1H), 8.25 (m, 1H), 7.92 (br s, 1H), 7.44 (m, 1H), 7.38 (m, 1H), 7.31 (m, 2H), 7.24 (m, 1H), 4.55 (m, 2H), 4.17 (q, 2H), 3.85 (dd, 1H), 3.70 (dd, 1H), 3.16 (d, 1H), 3.08 (d, 1H), 2.67 (m, 1H), 2.58 (m, 1H), 2.14 (br s, 1H), 1.63 (d, 1H), 1.37 (m, 5H), 1.01 (m, 1H).

3. A possible synthetic route towards compounds of the invention in which R³ is an ortho-monochloroaryl with an amide at the para-position is described in the scheme below (Scheme 13.). Commercially available acid 42 can be first reduced and subsequently reoxidized to aldehyde 44. After ringclosing reaction to the substituted purines, the bromide can be transformed to the acid which can be functionalized to e.g. an amide by procedures well known in the art (e.g. R—NH₂/TBTU/DIEA/DCM).

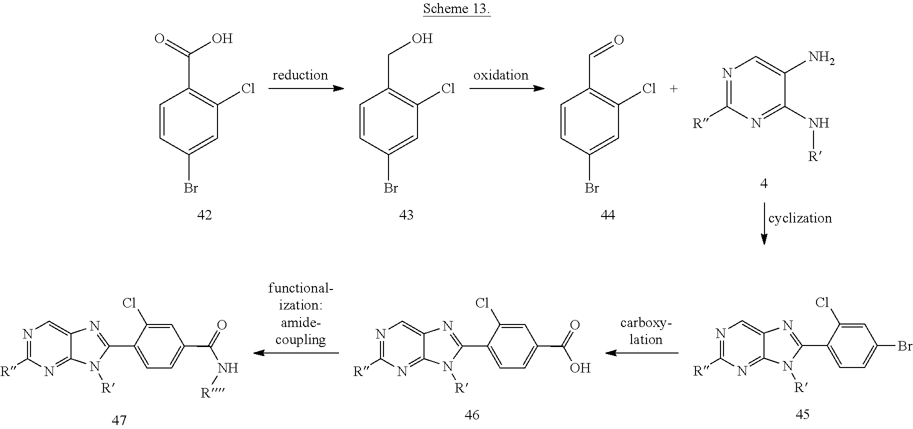

The following is an exemplary procedure for the preparation of a compound of the invention.

### Synthesis of 3-chloro-N-cyclohexyl-4-(2-(3,4-difluorobenzylamino)-9-(R)-piperidin-3-ylmethyl)-9H-purin-8-yl)benzamide 2,2,2-trifluoroacetate (629)

One possible process for the synthesis of 3-chloro-N-cyclohexyl-4-(2-(3,4-difluorobenzylamino)-9-((R)-piperidin-3-ylmethyl)-9H-purin-8-yl)benzamide (629) is shown in Scheme 14 below and detailed in the following description. Compound 13 is prepared in accordance with syntheses described before (Scheme 3 and corresponding procedures).

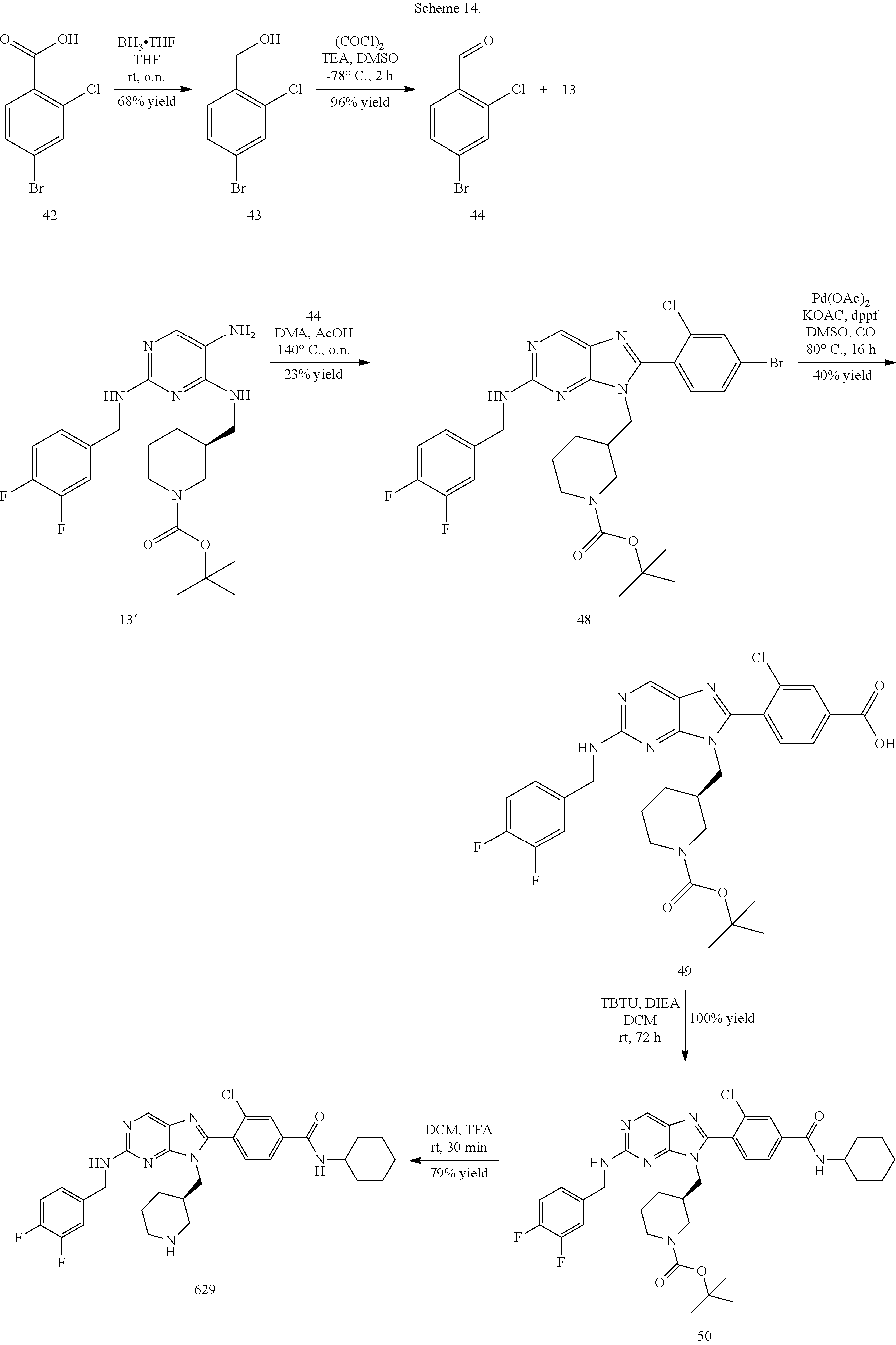

### Compound 43

To a −10° C. cooled solution of 4-bromo-2-chlorobenzoic acid 42 (14.4 g, 61 mmol, 1.0 equiv.) in THF (280 ml) was added dropwise a 1 M solution of BH₃.THF (91.4 ml, 1.5 equiv.), temperature was maintained at −10° C. The reaction mixture was stirred overnight to reach room temperature.

For workup, the mixture was added carefully to a solution of K₂CO₃ (4 g) in water (500 ml). The solution was stirred 15 minutes and concentrated in vacuo. The remaining water layer was diluted with EtOAc, washed with 1 N HCl and brine, dried on Na₂SO₄, filtered and concentrated in vacuo to obtain compound 43 in 68% yield (9.2 g, 41.5 mmol).

### Compound 44

Oxalylchloride (6.9 g, 54 mmol, 1.3 equiv.) was dissolved in DCM (153 ml) and cooled to −78° C. To the cooled solution was a solution of DMSO (4.72 ml, 66.5 mmol, 1.6 equiv.) in DCM (57 ml) added dropwise and stirred for 15 minutes at −78° C. Compound 43 (9.2 g, 41.5 mmol, 1.0 equiv.) was dissolved in DCM (116 ml) and added dropwise while the temperature was maintained at −78° C. The r.m. was stirred for 2 h at −78° C. Then TEA (28.7 ml, 207 mmol, 5 equiv.) was added and the mixture was allowed to reach room temperature. After stirring for 30 minutes at r.t., the reaction mixture was diluted with 300 ml DCM and washed with saturated NH₄Cl, brine, dried on Na₂SO₄, filtered and concentrated in vacuo. Compound 44 was obtained in 96% yield (8.8 g).

### Compound 48

To a solution of compound 13 (9 g, 0.020 mmol, 1.0 equiv.) in DMA (175 ml) was added compound 44 (8.8 g, 0.040 mmol, 2.0 equiv.) and acetic acid (17.5 ml). The reaction mixture was heated to 140° C. and stirred overnight while air was bubbled through. After 48 h the r.m. was cooled to r.t., diluted with EtOAc and extracted with water (5×), brine (2×), dried on Na₂SO₄, filtered and concentrated in vacuo. After purification by column chromatography (1:1 heptane:EtOAc), compound 48 was obtained in a 23% yield (3.0 g).

### Compound 49

To a mixture of KOAc (2.4 g, 24 mmol, 4.0 equiv.), Pd(OAC)₂ (148 mg, 0.66 mmol, 0.11 equiv.), and dppf (1.42 g, 2.56 mmol, 0.43 equiv.) under N₂-atmosphere, a solution of compound 48 (3.9 g, 6 mmol, 1.0 equiv.) in DMSO (110 mL) was added. By use of a gas balloon filled with CO(g) and a vacuum pump, the reaction mixture was kept under CO-atmosphere. The mixture was heated at 80° C. for 16 h. After cooling to r.t. and neutralizing by 0.5 M HCl, the product was extracted with DCM and washed with water (4×) and brine. After drying on Na₂SO₄, filtering and concentration in vacuo, the crude product was purified by column chromatography (5:95 MeOH:DCM to wash away impurities, 1:5:94 AcOH:MeOH:DCM to elute the product). After concentrating productfractions in vacuo, the product was coevaporated with toluene. Compound 49 was obtained in 40% yield.

### Compound 50

To a solution of compound 49 (40 mg, 0.065 mmol, 1.0 equiv.) in DCM (1 ml) was added a prestirred solution (r.t., 10 min.) of DIEA (0.057 ml, 0.33 mmol, 5.0 equiv), TBTU (31 mg, 0.098 mmol, 1.5 equiv.) and aminocyclopentane (0.019 ml, 0.20 mmol, 3.0 equiv.) in DCM (2 ml). R.m. was stirred at r.t. for 72 h.

R.m was poured out in sat. NaHCO₃ and extracted with EtOAc (2×). After washing with brine, drying on Na₂SO₄, filtering and concentrating in vacuo, crude compound 50 was obtained in 88% yield.

### 3-chloro-N-cyclohexyl-4-(2-(3,4-difluorobenzylamino)-9-((R)-piperidin-3-ylmethyl)-9H-purin-8-yl)benzamide 2,2,2-trifluoroacetate (629)

To a solution of compound 50 (45 mg, 0.065 mmol) in DCM (1 ml) was added TFA (0.5 ml). The reaction mixture was stirred at rt for 30 min. Rm was concentrated in vacuo and purified by prep-HPLC (0-50% ACN, with TFA). Productfractions were concentrated and lyophilized in ACN/H₂O to obtain the TFA-salt of compound 629 in 79% yield (36 mg).

TFA-salt: ¹H NMR (400 MHz, DMSO-D6), ppm: 8.70 (s, 1H), 8.53 (d, 1H), 8.50 (br s, 1H), 8.16 (s, 1H), 8.00 (d, 1H), 7.94 (br s, 1H), 7.78 (s, 1H), 7.75 (s, 1H), 7.43 (m, 1H), 7.37 (m, 1H), 7.24 (m, 1H), 4.53 (m, 2H), 3.91 (m, 1H), 3.81 (m, 1H), 3.70 (m, 1H), 3.11 (d, 1H), 2.95 (d, 1H), 2.62 (m, 1H), 2.45 (m, 1H), 2.04 (br s, 1H), 1.84 (m, 2H), 1.76 (m, 2H), 1.62 (m, 2H), 1.34 (m, 6H), 1.15 (m, 1H), 0.95 (m, 1H).

4. Another possible synthetic route towards compounds of the invention in which R³ is an ortho-monochloroaryl with an amide at the para-position is described in the scheme below (Scheme 15.).

The nitrogroup of 55 can be reduced by procedures well known in the art (e.g. Raney Ni). The primary amine can be functionalized to e.g. a reversed amide by procedures well known in the art (e.g. R—NH₂/TBTU/DIEA/DCM).

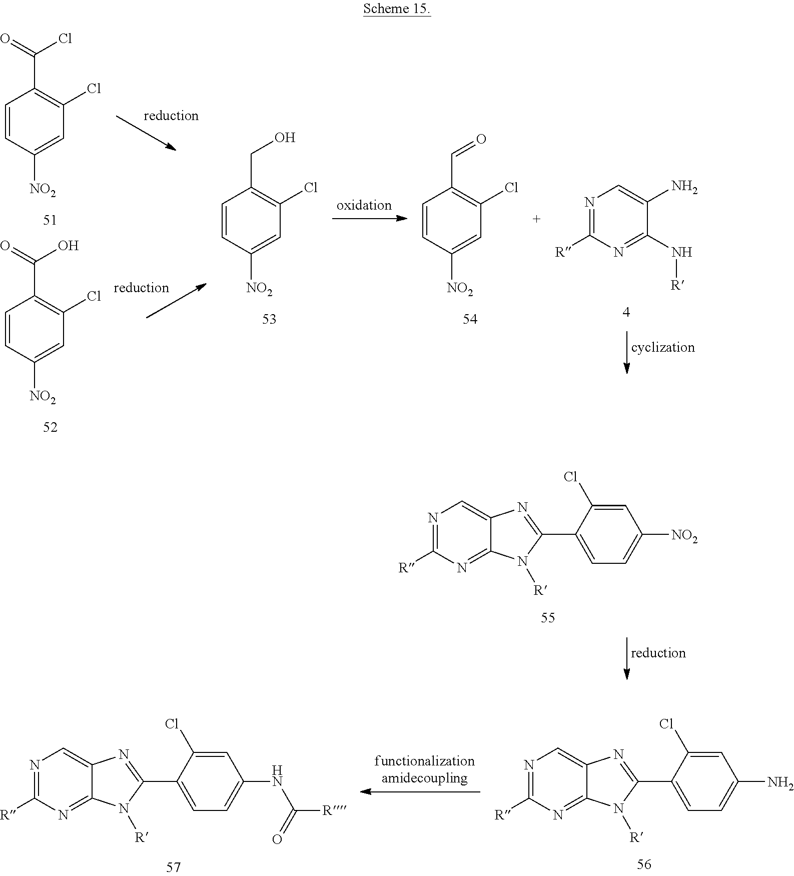

The following is an exemplary procedure for the preparation of a compound of the invention.

### Synthesis of 2-chloro-4-nitrobenzocarboxaldehyde (54) and N-(3-chloro-4-(2-(3,4-difluorobenzylamino)-94(R)-piperidin-3-ylmethyl)-9H-purin-8-yl)phenyl)acetamide 2,2,2-trifluoroacetate (547)

One possible process for the synthesis of compound 547 (Scheme 16) is detailed in the following description. Compound 13′ is prepared in accordance with syntheses described before (Scheme 3 and corresponding procedures).

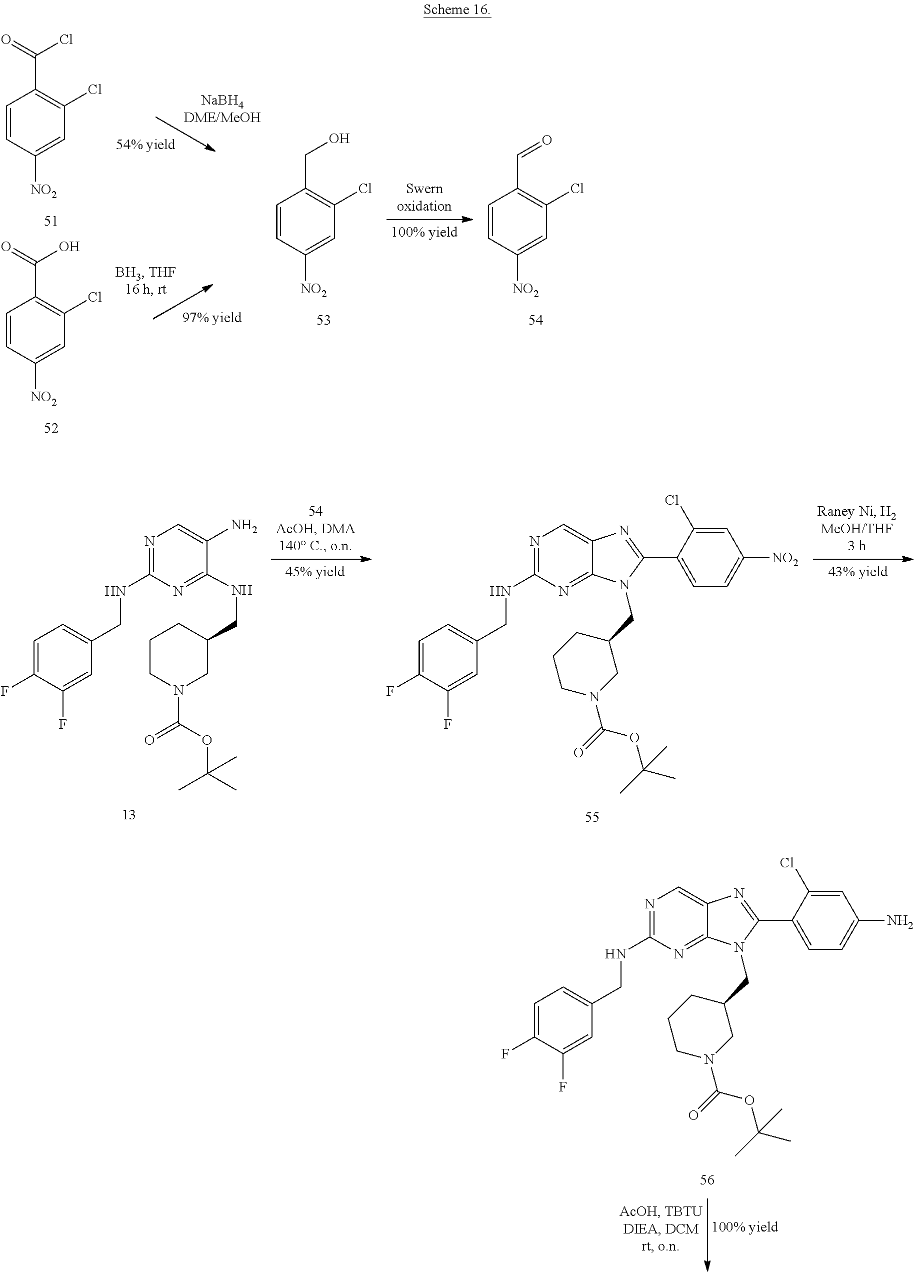

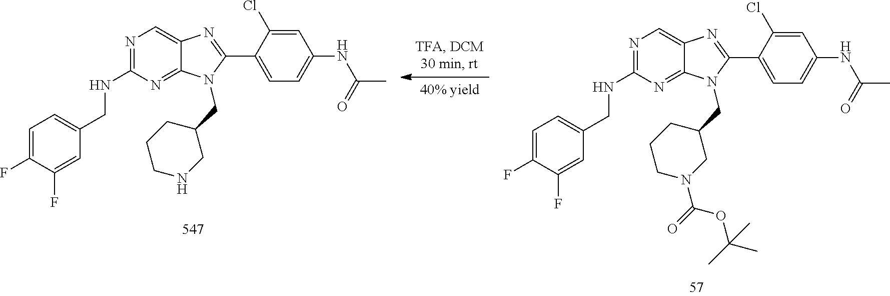

### Compound 53

Starting with acid chloride 51: 2-chloro-4-nitrobenzoylchloride 51 (6.22 g, 28.3 mmol, 1.0 equiv.) can be reduced by using NaBH₄ (1.1 g, 28.3 mmol, 1.0 equiv.) in a DME (30 mL)/MeOH (15 mL) mixture. After workup, product 53 was obtained in 54% yield (2.89 g).

Starting with acid 52: a solution of 2-chloro-4-nitrobenzoic acid (15.95 g, 79 mmol, 1.0 equiv.) in THF (200 mL) was cooled to 0° C. BH₃ (118.7 mL, 118.7 mmol, 1 M solution in THF, 1.5 equiv.) was added dropwise. Reaction mixture was allowed to warm to room temperature and stirred for 16 h. A sat'd solution of K₂CO₃ in water was added dropwise until gas evolution stopped. After precipitation of a white solid, the r.m. was filtered and washed with EtOAc. Filtrate and washings were combined and concentrated in vacuo. The product was redissolved in EtOAc, washed with 1N HCl (2×), sat'd NaHCO₃ and brine and dried on Na₂SO₄. After filtration and concentration in vacuo, compound 53 was obtained as a yellowish solid in 97% yield (14.45 g).

### Compound 54

A solution of oxalyl chloride (8.6 ml, 100 mmol, 1.3 equiv.) in DCM (250 ml) was cooled to −70° C. A solution of DMSO (8.9 ml, 125 mmol, 1.6 equiv.) in DCM (50 ml) was added slowly, maintaining temperature below −70° C. The mixture was stirred for 15 minutes. Compound 52 (14.45 g, 77 mmol, 1.0 equiv.) was dissolved in DCM (150 ml) and the solution was added dropwise to the mixture. After addition, the mixture was stirred at −70° C. for 45 minutes. Et₃N (54 mL, 385 mmol, 5.0 equiv.) was added to the mixture, then the mixture was allowed to warm to the room temperature and stirred overnight. The mixture was diluted with DCM (500 mL) and washed with sat'd NH₄Cl (2×), water and brine. After drying on Na₂SO₄, filtration and concentration in vacuo, compound 54 was obtained as a solid in a quantitative yield (14.29 g).

### Compound 55

To a solution of compound 13′ (13 g, 28.9 mmol, 1.0 equiv.) in DMA (200 ml) was added AcOH (30 ml) and aldehyde 54 (8.7 g, 46.8 mmol, 1.6 equiv.). The reaction mixture was heated to 140° C. overnight with air bubbling through the reaction mixture. After completion, rm was cooled to rt, diluted with EtOAc and washed with water (3×) and brine. After drying on Na₂SO₄, filtration and concentration in vacuo, the crude product was purified by column chromatography (5% MeOH/95% DCM). Compound 55 was obtained in a 45% yield (8.13 g).

### Compound 56

To a solution of compound 55 (8.13 g, 13.24 mmol) in MeOH (100 ml) and THF (100 ml) was added Raney Ni under N₂-atmosphere. The rm was stirred under H₂-atmosphere for 3 h. The mixture was filtered over celite and concentrated in vacuo. The crude product was redissolved in DCM, some impurities remaining insoluble. After filtration, the filtrate was purified using column chromatography (5% MeOH/95% DCM). The product was purified again by dissolving in DCM and reprecipitatation by heptane. The supernatant was separated and the product was dried under vacuum. Compound 56 was obtained in a 43% yield (3.3 g).

### Compound 57

To a solution of AcOH (4.94 μl, 0.086 mmol, 1.0 equiv.) in DCM (2 ml) was added TBTU (41.2 mg, 0.128 mmol, 1.5 equiv.) and DIEA (45 μl, 0.257 mmol, 3.0 equiv.). Rm was stirred at rt for 10 minutes. To this mixture was added a solution of compound 56 (50 mg, 0.086 mmol, 1.0 equiv.) in DCM (1 ml). The rm was stirred at r.t. overnight.

Extra TBTU and acetic acid (2 equiv.) were needed to complete the reaction over 72 h. The r.m. was poured out in sat'd NaHCO₃ and extracted with EtOAc (2×). After washing with brine, drying on Na₂SO₄, filtration and concentration in vacuo, the crude product was purified by column chromatography (DCM:MeOH 9:1). Compound 57 was obtained in a 100% yield (53 mg).

### N-(3-chloro-4-(2-(3,4-difluorobenzylamino)-9-((R)-piperidin-3-ylmethyl)-9H-purin-8-yl)phenyl)acetamide 2,2,2-trifluoroacetate (547)

To a solution of compound 57 (53 mg, 0.086 mmol) in DCM (1 ml) was added TFA (0.5 ml). Rm was stirred at rt for 30 minutes. The reaction mixtured was concentrated in vacuo and the crude product was purified by prep-HPLC (0-50% ACN with TFA). Product fractions were concentrated and lyophilized to obtain the TFA-salt of compound 547 in a 40% yield (22 mg).

TFA-salt: ¹H NMR (400 MHz, DMSO-D6), ppm: 10.42 (s, 1H), 8.72 (s, 1H), 8.52 (br d, 1H), 8.16 (m, 1H), 8.03 (m, 1H), 7.92 (br s, 1H), 7.63 (d, 1H), 7.57 (d, 1H), 7.43 (m, 1H), 7.37 (m, 1H), 7.24 (br s, 1H), 4.53 (m, 2H), 3.91 (m, 1H), 3.80 (m, 1H), 3.11 (br d, 1H), 2.94 (br d, 1H), 2.65 (m, 1H), 2.44 (m, 1H), 2.12 (s, 3H), 2.03 (br s, 1H), 1.62 (m, 1H), 1.36 (m, 2H), 0.96 (m, 1H).

5. Another possible synthetic route towards compounds of the invention in which R³ is an ortho,ortho-dichloroaryl with an amide at the para-position is described in the scheme below (Scheme 17.).

After ringclosing reaction to the purines, the Cbz-N-group can be deprotected by procedures well known in the art (e.g. Pd/C/H₂). The primary amine can be functionalized to e.g. a carbamate or reversed amide by procedures well known in the art (e.g. R—NH₂/TBTU/DIEA/DCM).

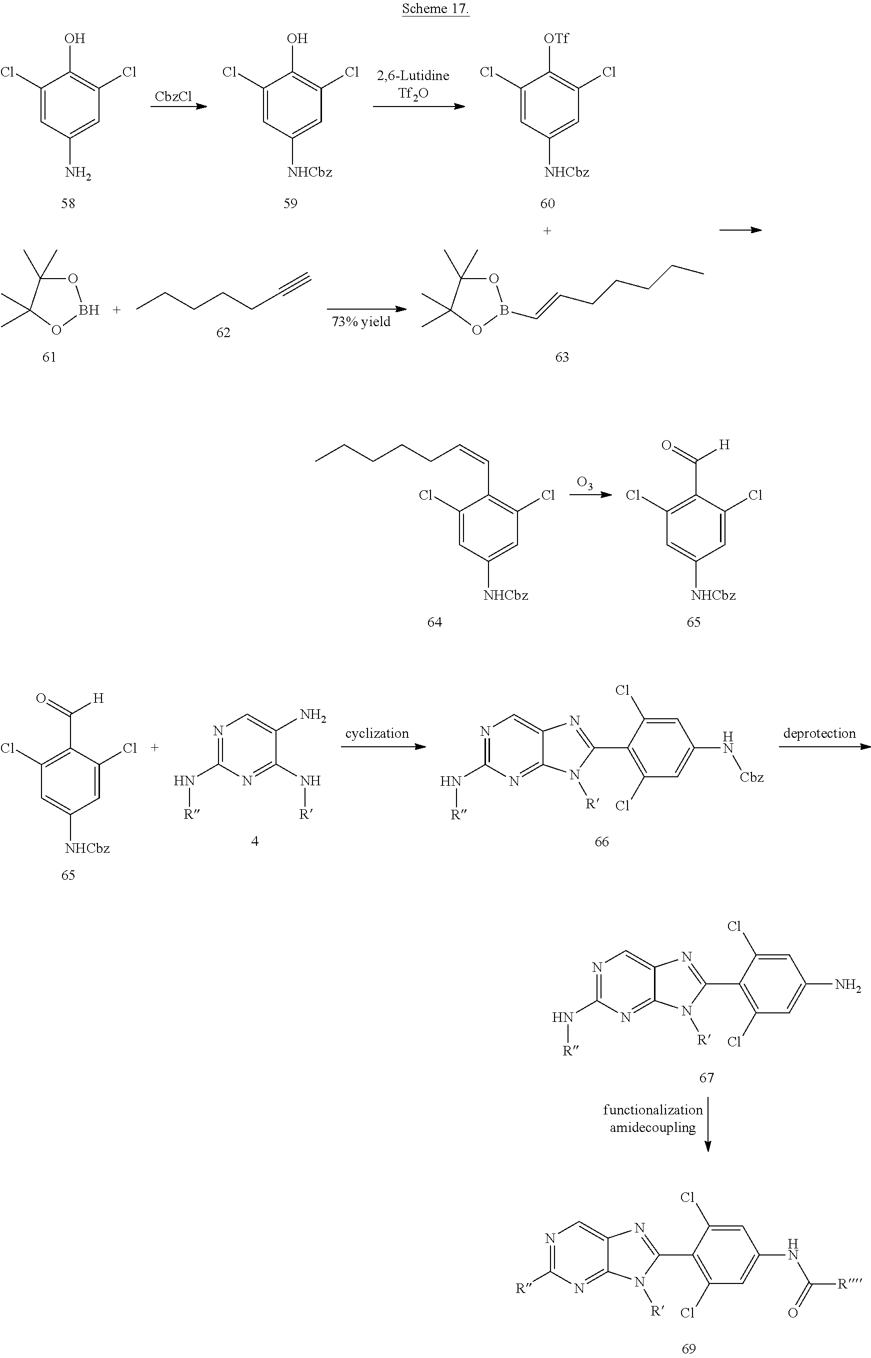

The following is an exemplary procedure for the preparation of a compound of the invention.

### Synthesis of benzyl-3,5-dichloro-4-formylphenylcarbamate (65) and (R)—N-(3,5-dichloro-4-(2-(3,4-difluorobenzylamino)-9-(piperidin-3-ylmethyl)-9H-purin-8-yl)phenyl)acetamide 2,2,2-trifluoroacetate (553)

One possible process for the synthesis of compound 553 (Scheme 18) is detailed in the following description. Compound 13′ is prepared in accordance with syntheses described before (Scheme 3 and corresponding procedures).

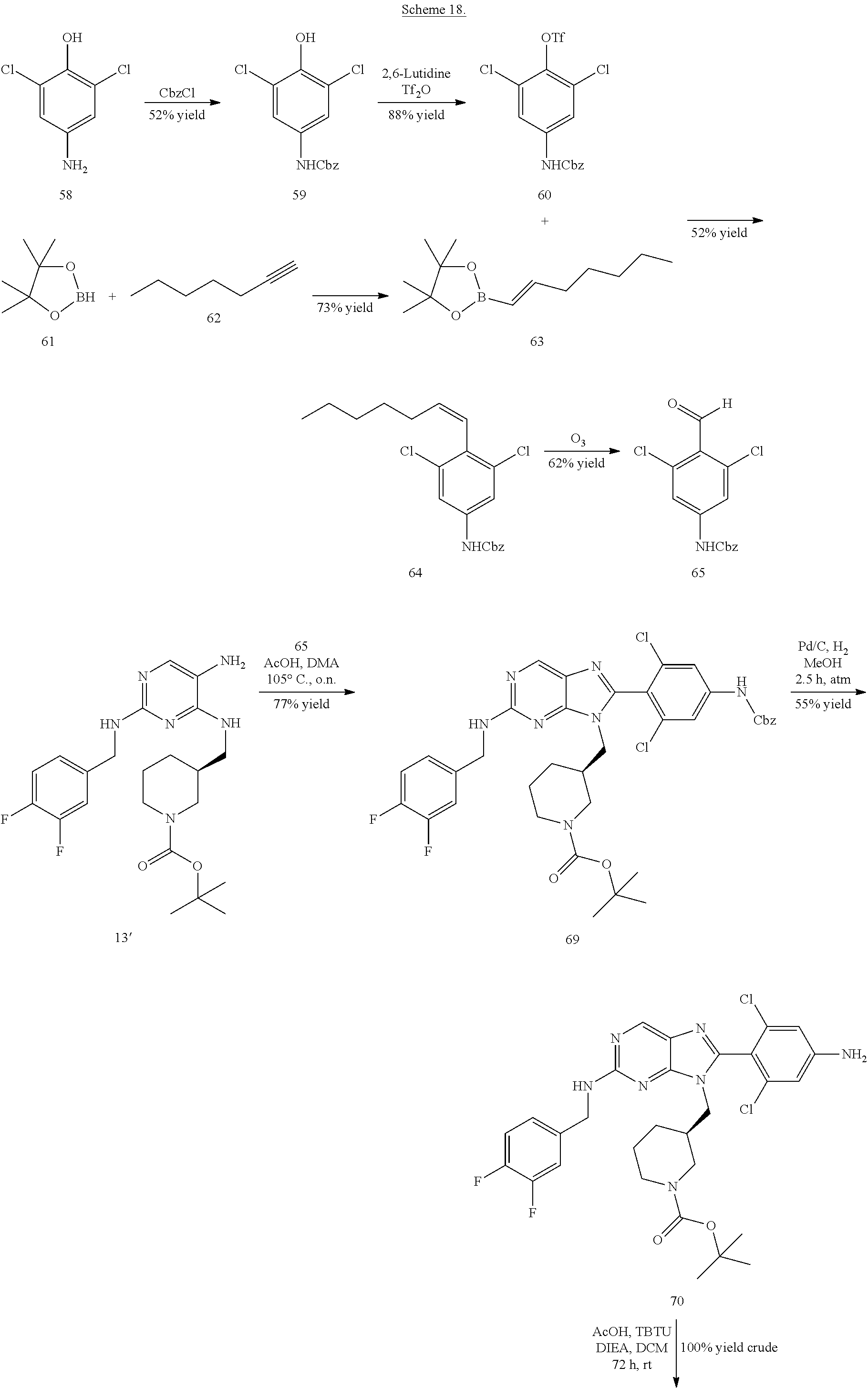

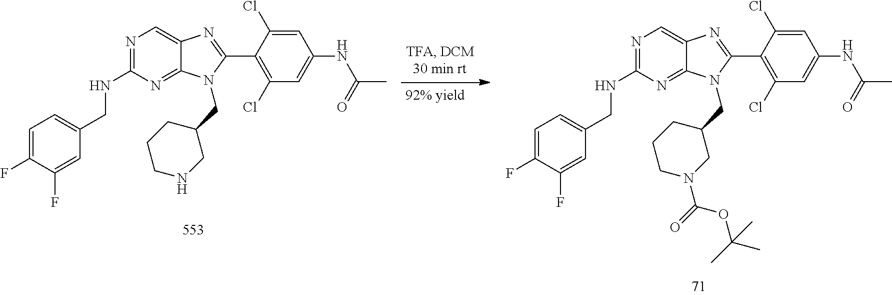

### Compound 59

To a solution of compound 58 (86.7 g, 0.49 mol, 1.0 equiv.) in THF (2 L) at 0° C. was added CbzCl (70 ml, 0.49 mol, 1.0 equiv.) dropwise and stirred mechanically. The mixture was stirred overnight at room temperature. The mixture was filtered, stirred with EtOAc/heptane and filtered again. The mother liquor was stirred with 200 ml of triethylamine for 3 h. The filtered solid was also added to the mixture and it was stirred overnight. The mixture was concentrated, NaHCO₃-sat'd was added, extraction with EtOAc and concentration in vacuo. To lose the disubstituted (bis-CBz) byproduct, the mixture was redissolved in THF and 4N NaOH (200 mL) was added. The mixture was stirred at 50° C. overnight and cooled to rt. The mixture was acidified to pH=3 and extracted with EtOAc (3×). The combined organic layers were washed with sat'd NaHCO₃ and brine and dried over Na₂SO₄. After filtration and concentration in vacuo, the solid was dissolved in DCM and precipitated with heptane to afford the CBz-protected aminophenol 59 in 52% yield (80 g).

### Compound 60

To a solution of compound 59 (80 g, 256 mmol, 1.0 equiv.) in DCM (1 L) was added 2,6-lutidine (60.5 g, 564 mmol, 2.2 equiv.). The mixture was cooled to −78° C. Triflic anhydride (86.8 g, 307 mmol, 1.2 equiv.) was added dropwise while keeping the temperature below −75° C. The reaction mixture was stirred overnight at room temperature. After completion, the reaction mixture was diluted with TBME and washed with water (3×), brine, dried on Na₂SO₄, filtered and concentrated in vacuo.

The crude material was purified by column chromatography (heptane:EtOAc 9:1) to yield 88% (91.5 g) of compound 60.

### Compound 63

A mixture of 1-heptyne 62 (75 g, 777 mmol, 2.0 equiv.) and pinacolborane 61 (49.7 g, 388 mmol, 1.0 equiv.) was stirred overnight at 70° C. Rm was concentrated in vacuo (evaporation of unreacted 1-heptyne and pinacolborane) to yield compound 63 in 43% yield. Unreacted compounds 61 and 62 were stirred again for two days at 80° C. After concentration in vacuo compound 63 was obtained. Combining both batches gave an overall yield of 73% (63.6 g).

### Compound 64

Compound 60 (54 g, 122 mmol, 1.0 equiv.) and compound 63 (30 g, 134 mmol, 1.1 equiv.) were dissolved in DME. A solution of Na₂CO₃ (39 g, 366 mmol, 3.0 equiv.) in water (70 ml) was added and the mixture was degassed (3×) and put under N₂-atmosphere. Pd(PPh₃)₄ (2.6 g, 2.5 mmol, 0.02 equiv.) was added. The reaction was stirred for 72 h at 70° C. After completion, the mixture was filtered over Celite and washed with water and EtOAc. The filtrate was extracted with EtOAc (3×). Combined organic layers were washed with brine, dried on Na₂SO₄, filtered and concentrated in vacuo. After purification by column chromatography (heptane:EtOAc 9:1) compound 64 was obtained in 52% yield (25 g).

### Benzyl-3,5-dichloro-4-formylphenylcarbamate (65)

A solution of compound 64 (9.4 g, 24 mmol) in DCM (200 ml) at −78° C. was bubbled through with ozone until a blue color appeared. This color maintained for 5 minutes. The rm was flushed with nitrogen for approximately 20 minutes. DMS (7.4 g, 120 mmol, 5.0 equiv.) was added and the rm was stirred o.n. at rt.

Water was added and the organic layer was extracted with water (3×). The water layers were collected and extracted with EtOAc/THF (3×). Combined organic layers were washed with brine, dried on Na₂SO₄, filtered and concentrated in vacuo.

The product was crystallized by EtOAc/heptane. After filtration and drying, compound 65 was obtained in 62% yield (4.8 g).

### Compound 69

To a solution of compound 13′ (1.70 g, 3.8 mmol) in DMA (20 ml) was added compound 65 (2.46 g, 7.60 mmol, 2.0 equiv.) and AcOH (3.26 ml, 57.0 mmol, 15 equiv.). Rm was stirred o.n. at 105° C. in a open flask. After completion, the reaction mixture was cooled to rt and extracted with EtOAc (2×). After washing with water (2×) and brine, the crude product was dried on Na₂SO₄ and concentrated in vacuo. The crude product was purified by column chromatography (100% heptaan to 100% EtOAc). Product fractions were concentrated in vacuo to obtain compound 69 in 77% yield (2.20 g).

### Compound 70

Compound 69 (2.09 g, 2.78 mmol, 1.0 equiv.) was dissolved in methanol (100 ml). Pd/C (0.164 g, 0.139 mmol, 0.05 equiv.) was added. The reaction mixture was stirred under H₂-flow for 4.5 h. Pd/C was filtered off over Celite, Celite was washed with EtOAc. The filtrate was concentrated in vacuo. After purification by column chromatography (heptaan:EtOAc 4:1 to pure EtOAc) compound 70 was obtained in 55% yield (958 mg).

### Compound 71

To a solution of acetic acid (5.09 μl, 0.089 mmol, 1.1 equiv.) in DCM (2 ml) was added DIEA (56.3 μl, 0.323 mmol, 4.0 equiv.) and TBTU (36.3 mg, 0.113 mmol, 1.4 equiv.). Rm was stirred at rt for 10 min. A solution of compoundxxx in DCM (1 ml) was added to this mixtured. Rm was stirred at it for 4 h. Extra acetic acid, TBTU, DIEA and a few drops of DMF were added. Rm was stirred at it for 72 h. After completion, water was added to the r.m. The r.m. was extracted with DCM, washed with brine and concentrated in vacuo to obtain compound 71.

### (R)—N-(3,5-dichloro-4-(2-(3,4-difluorobenzylamino)-9-(piperidin-3-ylmethyl)-9H-purin-8-yl)phenyl)acetamide 2,2,2-trifluoroacetate (553)

To a solution of compound xxx in DCM (1 ml) was added TFA (0.2 ml). R.m. was stirred at it for 30 minutes. After concentration in vacuo, the crude product was purified by prep-HPLC (0-70% ACN, with TFA). Productfractions were concentrated in vacuo, lyophilization in ACN/H₂O obtained compound 553 as the TFA-salt in 92% yield (50 mg).

TFA-salt: ¹H NMR (400 MHz, DMSO-D6), ppm: 10.60 (s, 1H), 8.62 (br d, 1H), 8.27 (br d, 1H), 7.96 (br s, 1H), 7.93 (s, 1H), 7.88 (s, 1H), 7.44 (m, 1H), 7.38 (m, 1H), 7.25 (m, 1H), 4.53 (m, 2H), 3.86 (m, 1H), 3.72 (m, 1H), 3.15 (br d, 1H), 3.08 (br d, 1H), 2.67 (m, 1H), 2.57 (m, 1H), 2.14 (s, 3H), 2.10 (m, 1H), 1.63 (br d, 1H), 1.38 (m, 2H), 1.01 (m, 1H).

6. Another possible route for preparing purine analogs of the invention in which R2 is a linear (3C-5C) substituted amine is shown in Scheme 19. The most reactive chloride in the 2-position of pyrimidine 1 is first displaced by TBDMSO(C3-C5)NH₂ to yield compound 72. The chloride at the 4-position is then substituted with NH₂—R′. Reduction of the nitro group in 73 to an amine (74) using reagents well known in the art (e.g. Na₂S₂O₄/NH₄OH/H₂O/dioxane, Pd(C)/H₂/MeOH), followed by cyclization with an aryl aldehyde gives TBDMS-deprotected purine 75. Conversion to the mesylate 76 and subsequent reaction with secondary amines can lead to purines 77.

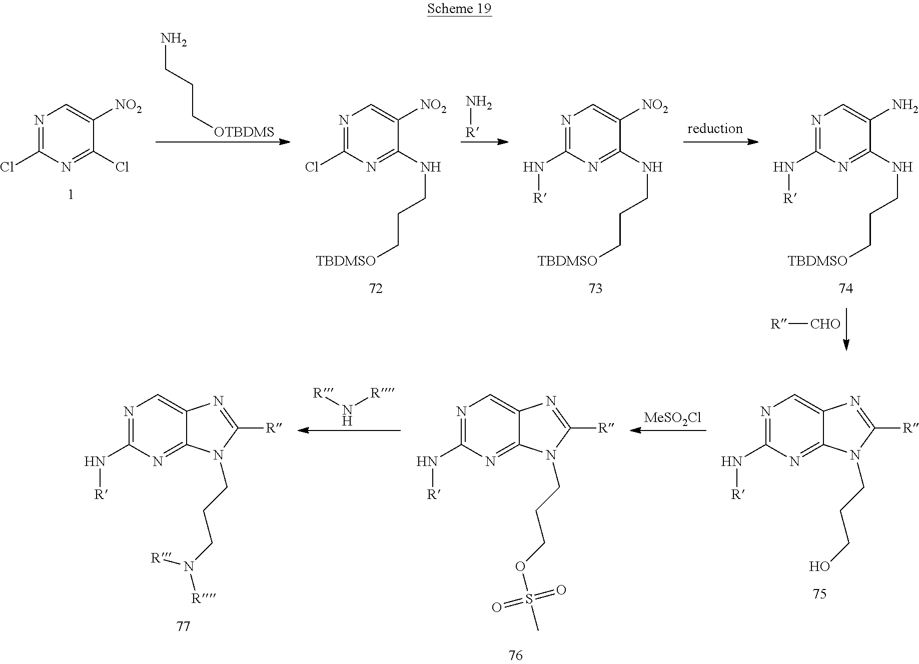

PKC-Theta IMAP Assay I

The activity of the compounds described in the present invention may be determined by the following procedure. This procedure describes a kinase assay that measures the phosphorylation of a fluorescently-labeled peptide by full-length human recombinant active PKCθ via fluorescent polarization using commercially available IMAP reagents.

The PKCθ used is made from full-length, human cDNA (accession number LO1087) with an encoded His-6 sequence at the C-terminus. PKCθ is expressed using the baculovirus expression system. The protein is purified with Ni-NTA affinity chromatography yielding a protein with 91% purity.

The substrate for this assay is a fluorescently-labeled peptide having the sequence LHQRRGSIKQAKVHHVK (FITC)-NH₂. The stock solution of the peptide is 2 mM in water.

The IMAP reagents come from the IMAP Assay Bulk Kit, product #R8063 or #R8125 (Molecular Devices, Sunnyvale, Calif.). The kit materials include a 5×IMAP Binding Buffer and the IMAP Binding Reagent. The Binding Solution is prepared as a 1:400 dilution of IMAP Binding Reagent into the 1×IMAP Binding Buffer.

The substrate/ATP buffer for this assay consists of 20 mM HEPES, pH 7.4 with 5 mM MgCl₂, and 0.01% Tween-20. Additionally, the buffer contains 100 nM substrate, 20 μM ATP, and 2 mM DTT which are added fresh just prior to use. The kinase buffer containing the PKCθ consists of 20 mM HEPES, pH 7.4 with 0.01% Tween-20. This buffer also contains 0.2 ng/μL PKCθ and 2 mM DTT which are added fresh just prior to use.

The plates used are Corning 3710 (Corning Incorporated, Corning, N.Y.). These are non-treated black polystyrene, 384-well with flat-bottoms. The serial dilutions are performed Nunc V-bottom 96-well plates.

The assay procedure starts the preparation of stock solutions of compounds at 10 mM in 100% DMSO. The stock solutions and the control compound are serially diluted 1:3.16 a total of 11 times into DMSO (37 μL of compound into 80 μL of DMSO). After the serial dilution has been completed, a further dilution is performed by taking 4 μL compound and adding to 196 μL substrate/ATP Buffer. Then, 10 μL aliquots of the compounds are transferred to the Costar 3710 plate. The kinase reaction is initiated by the addition of 10 μL PKCθ. This reaction is allowed to incubate for 1 hour at ambient temperature. The reaction is then quenched by the addition of 60 μL of Binding Solution. The plate is incubated for an additional 30 minutes at ambient temperature. The assay is measured using an Acquest™ Ultra-HTS Assay Detection System (Molecular Devices) in fluorescence polarization mode using 485 nm excitation and 530 nm emission.

### PKC-Theta IMAP Assay II

The activity of the compounds of the present invention is determined by the following procedure. This procedure describes a kinase assay that measures the phosphorylation of a fluorescently-labeled peptide by full-length human recombinant active PKCθ via fluorescent polarization using commercially available IMAP reagents.

The PKCθ used is made from full-length, human cDNA (accession number LO1087) with an encoded His-6 sequence at the C-terminus. PKCθ is expressed using the baculovirus expression system. The protein is purified with Ni-NTA affinity chromatography yielding a protein with ˜70% purity.

The substrate for this assay is a fluorescently-labeled peptide having the sequence LHQRRGSIKQAKVHHVK (FITC)—NH₂. The stock solution of the peptide is 0.06M in MilliQ water.

The IMAP reagents originate from the IMAP buffer kit with Progressive Binding System, product #R8127 (Molecular Devices, Sunnyvale, Calif.). The Binding Solution is prepared as a 1:400 dilution of IMAP Progressive Binding Reagent into the 1× buffer A IMAP Binding Buffer.

The kinase reaction buffer for this assay consists of 10 mM Tris-HCl, 10 mM MgCl₂, 0.01% Tween-20, 0.05% NaN₃, pH 7.2, and 1 mM DTT (freshly added prior to use).

The plates used are Black 384-F Optiplates (product #6007279, Packard).

The assay procedure starts with the preparation of serial dilutions of the compounds stored in 100% DMSO. The compounds are 10 times serially diluted 1:3.16, resulting in a final compound concentration range from 10 μM to 0.316 nM. All reagent solutions are prepared in kinase reaction buffer.

To 5 μl compound solution (4% DMSO), 5 μl of an ATP solution of 40 μM is added to the well. Subsequently, 5 μl of a 200 nM substrate solution is added. The kinase reaction is initiated by the addition of 5 μl PKCθ solution of 40 ng/ml. This reaction is allowed to incubate for 1 hour at ambient temperature. The reaction was stopped by adding 40 μl of IMAP Progressive Binding Solution. The plate is incubated for an additional 60 minutes at ambient temperature in the dark. Fluorescence polarization is measured using an Envision Multilabel reader (Perkin Elmer) in fluorescence polarization mode using 485 nm excitation and 530 nm emission.

Table 1 illustrates several examples of the compounds of the invention. These compounds are synthesized using one of the suitable procedures described above. The molecular weight of the compounds is confirmed by mass spectroscopy (m/z). The compounds of Table 1 are tested using one of the above-described PKCθ IMAP assays. Entries in the 100, 200, 300 and 400 series are tested using PKC-theta IMAP assay 1 and Entries in the 500, 600 and 700 are tested using PKC-theta IMAP assay II.

All compounds in Table 1 below exhibit PKCθ IMAP assay IC₅₀ values less than 10 μM. Entries in the 100 and 500 series exhibit IC₅₀ values less than 100 nM; entries in the 200, 300 and 600 series exhibit IC₅₀ values less than 1 μM; and entries in the 400 and 700 series exhibit IC₅₀ values less than 10 μM.

TABLE 1 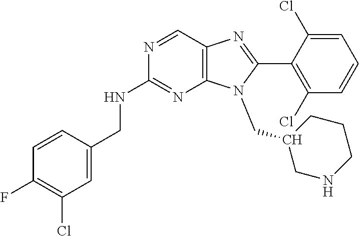
100 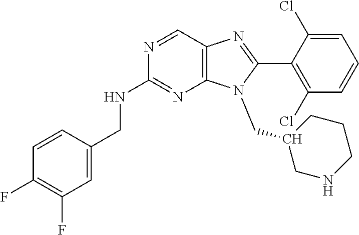
101 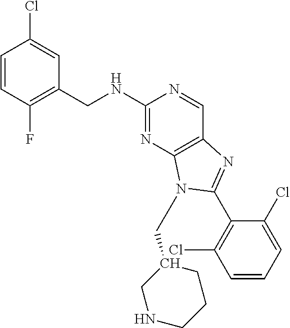
102 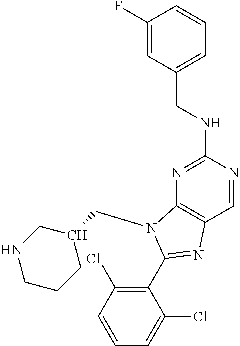
103 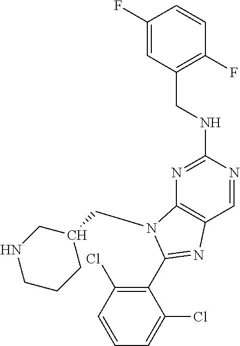
104 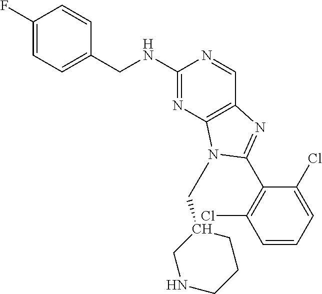
105 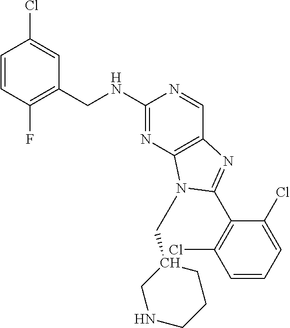
106 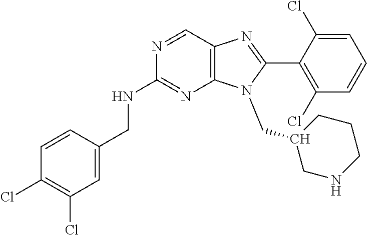
107 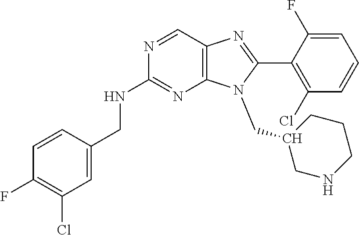
108 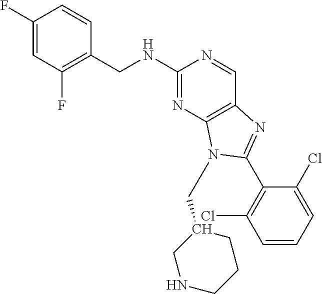
109 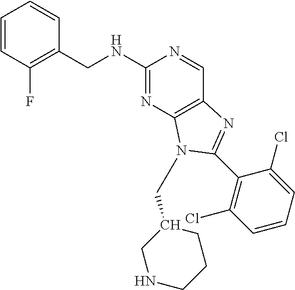
110 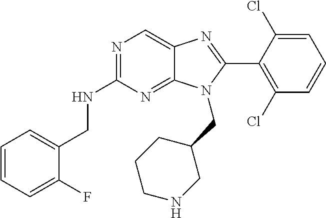
111 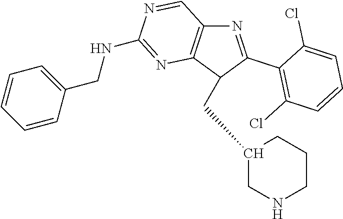
112 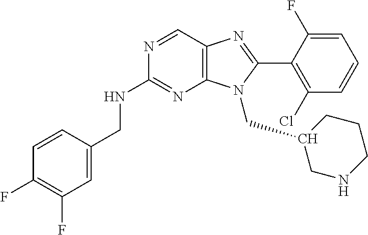
113 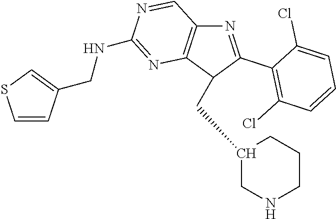
114 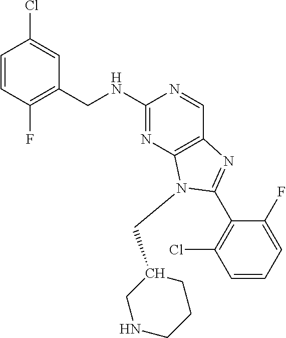
115 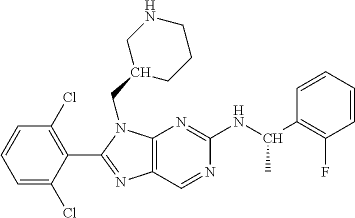
116 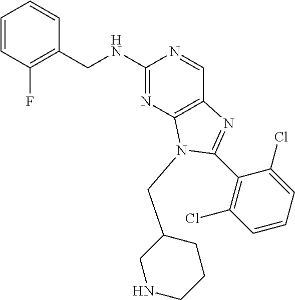
117 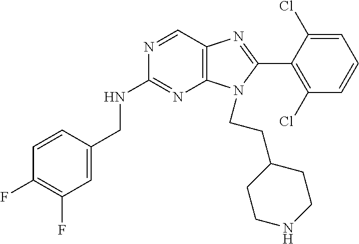
118 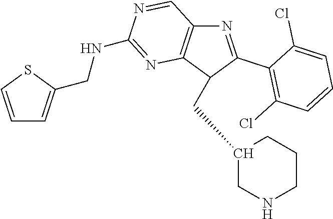
119 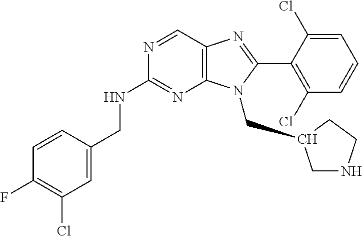
120 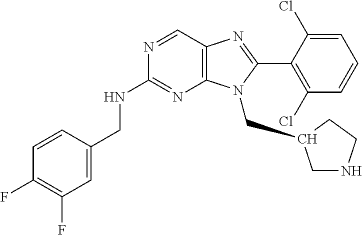
121 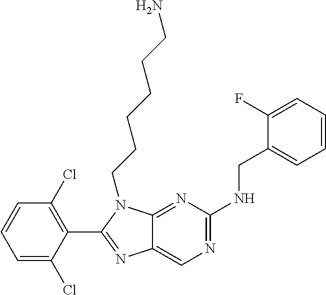
122 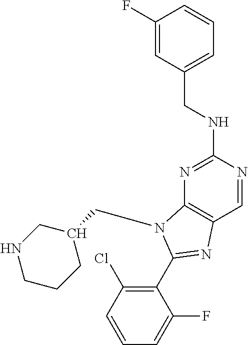
123 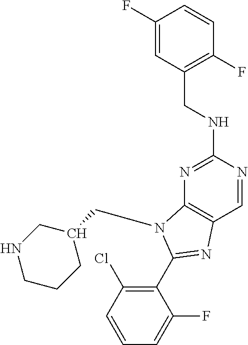
124 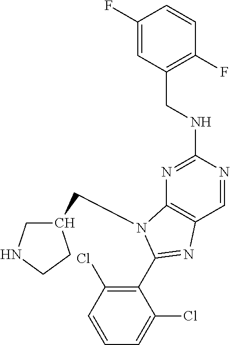
125 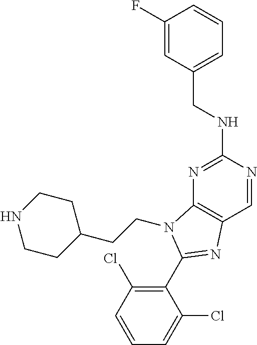
126 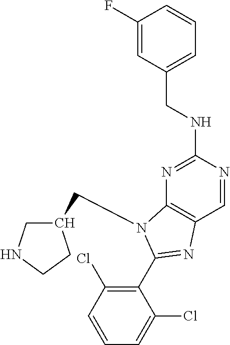
127 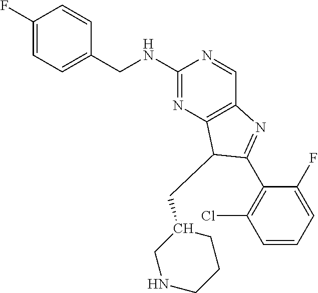
128 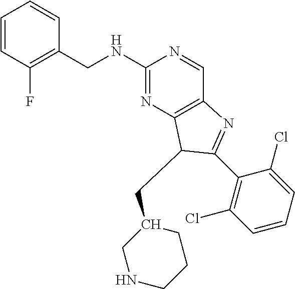
129 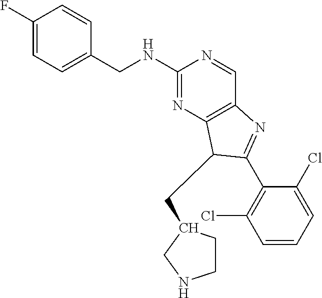
130 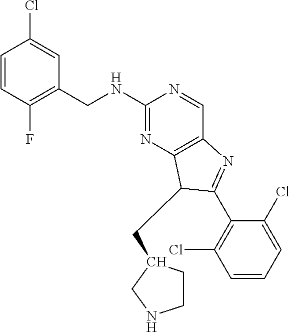
131 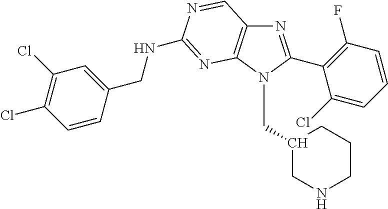
132 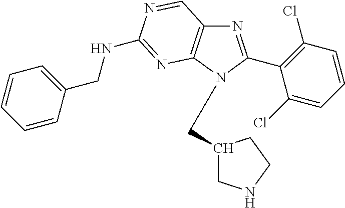
133 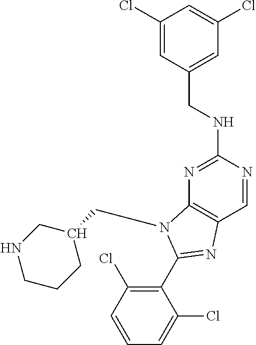
134 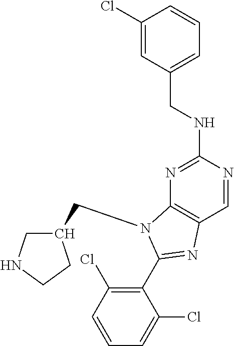
135 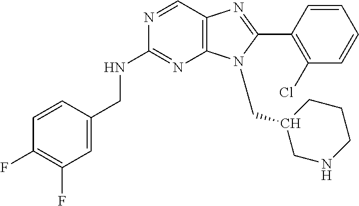
136 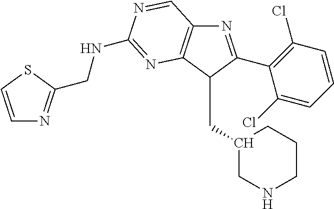
137 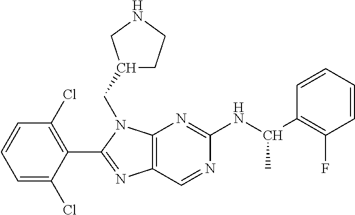
138 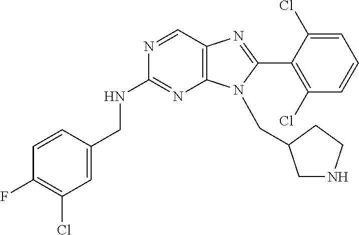
139 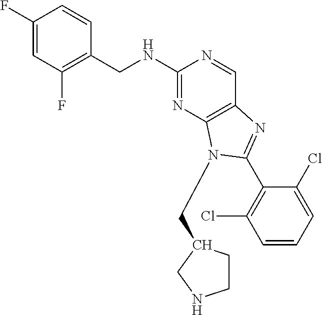
140 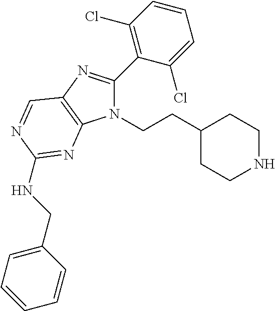
141 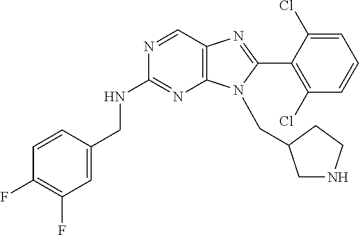
142 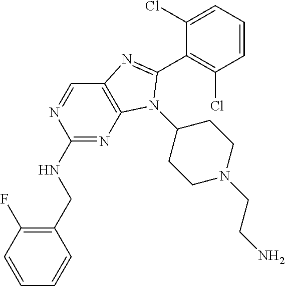
143 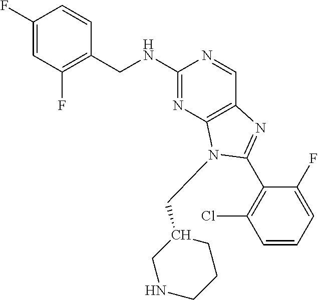
144 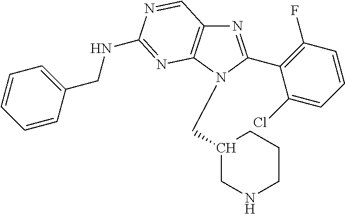
145 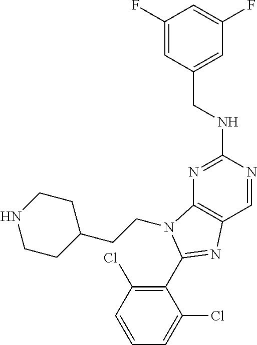
146 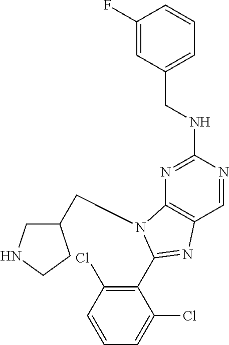
147 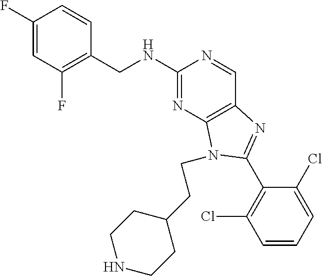
148 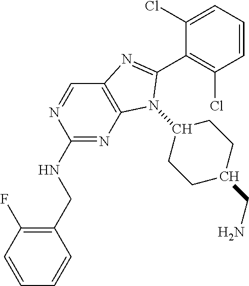
149 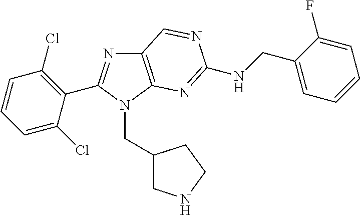
150 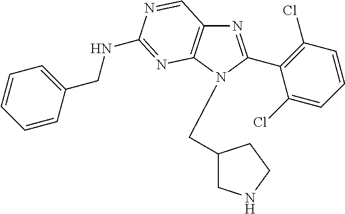
151 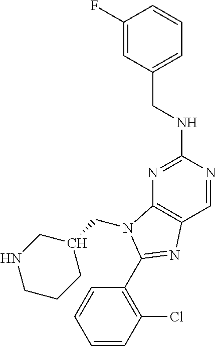
152 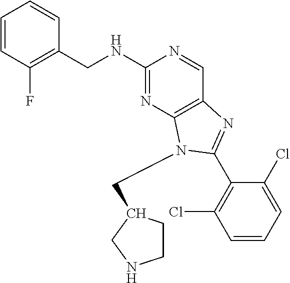
153 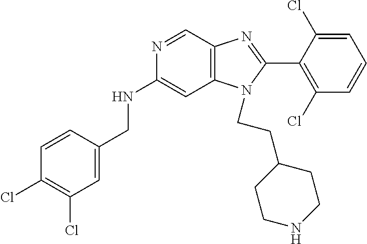
154 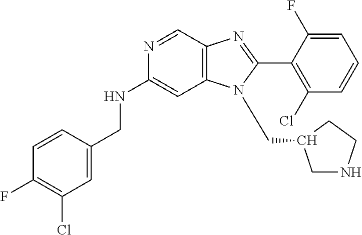
155 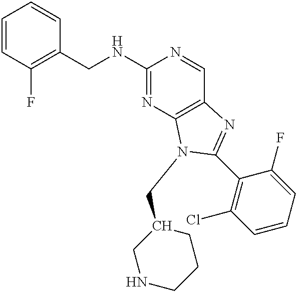
156 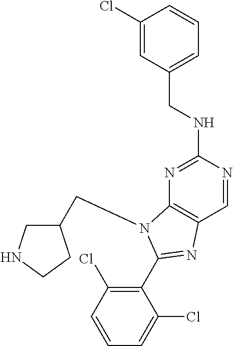
157 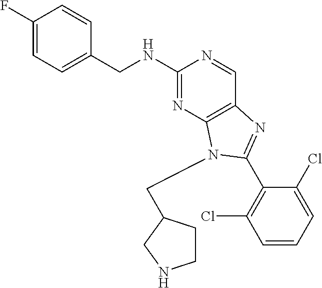
158 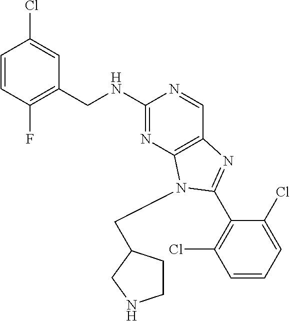
159 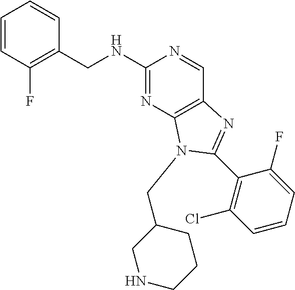
160 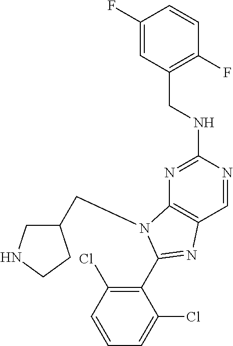
161 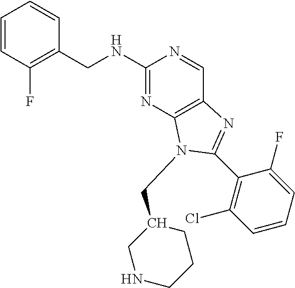
162 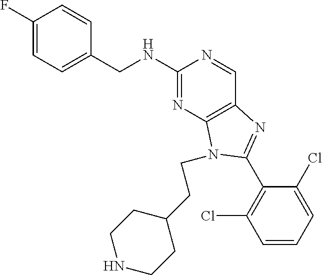
163 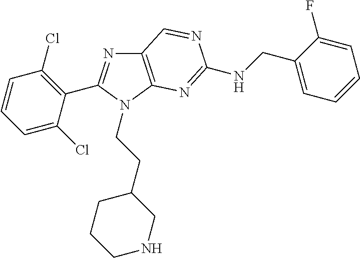
164 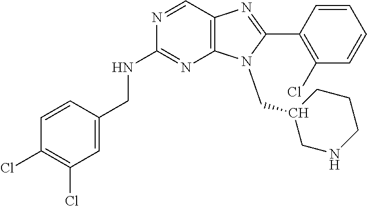
165 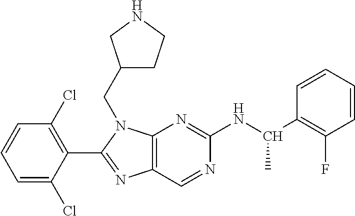
166 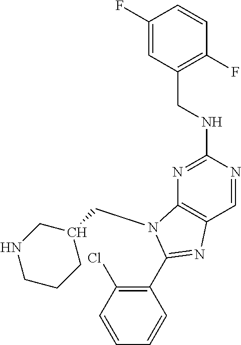
167 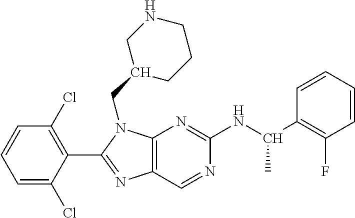
168 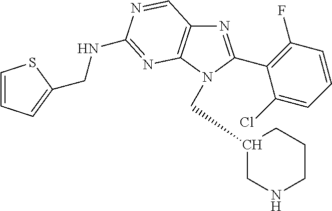
169 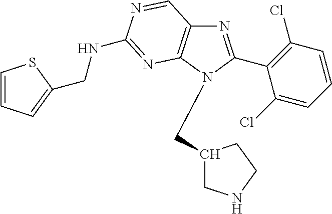
170 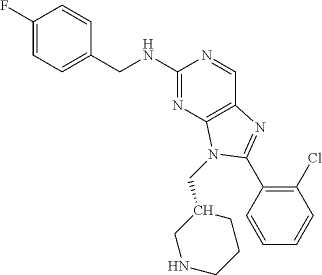
171 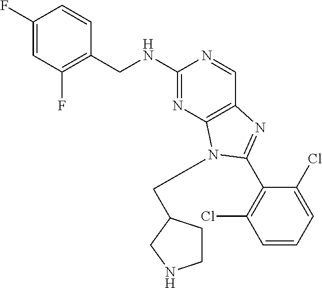
172 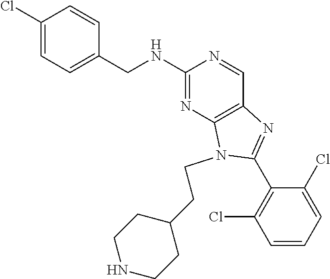
173 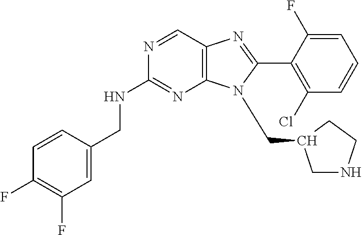
174 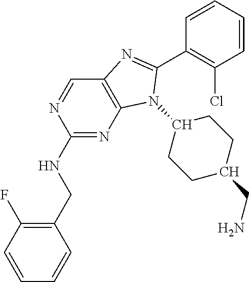
175 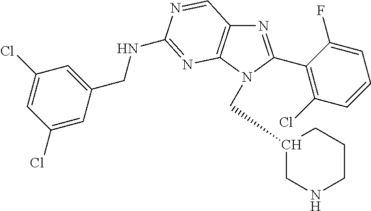
200 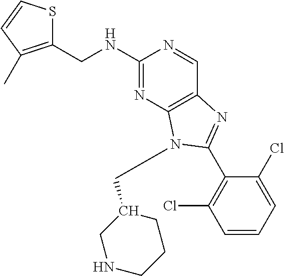
201 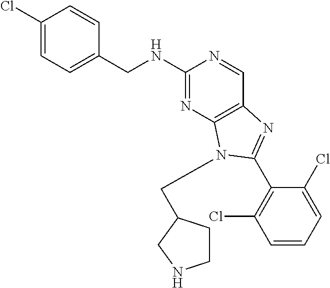
202 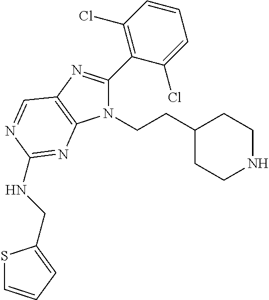
203 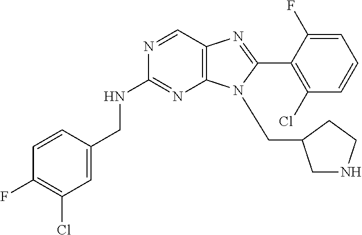
204 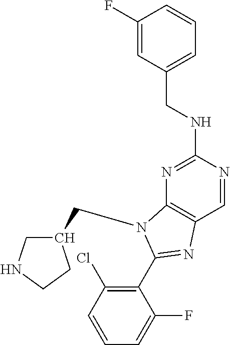
205 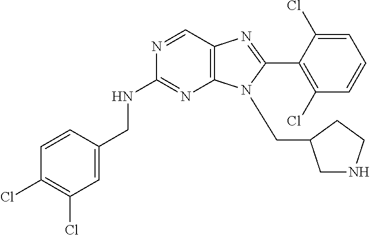
206 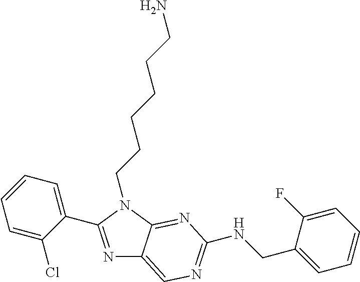
207 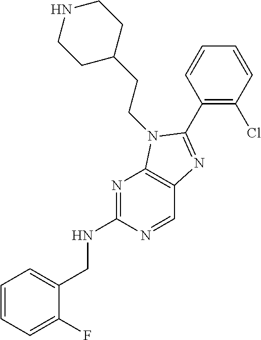
208 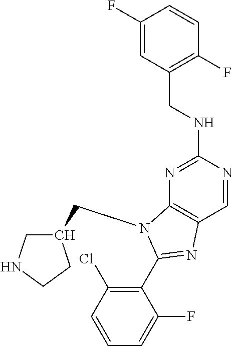
209 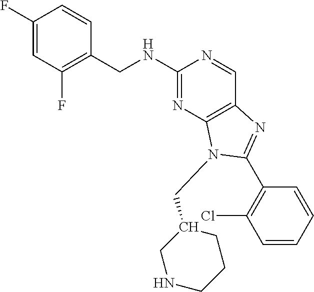
210 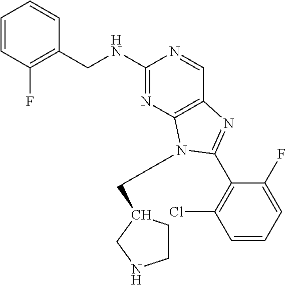
211 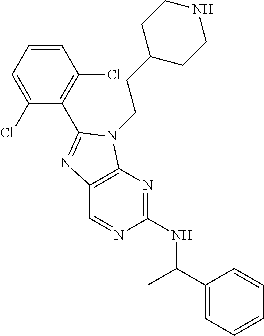
212 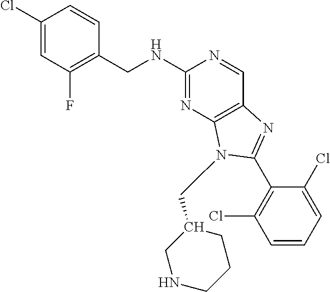
213 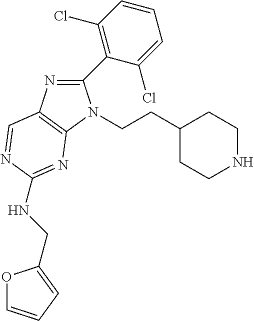
214 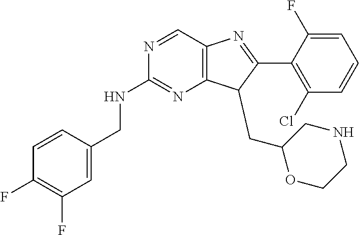
215 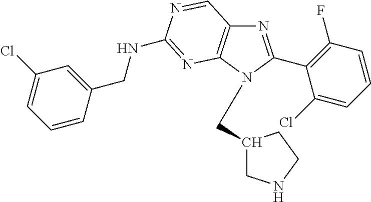
216 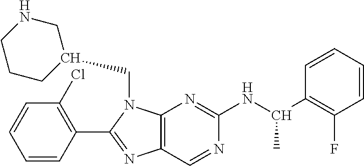
217 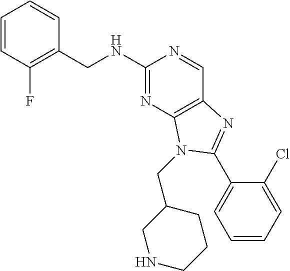
218 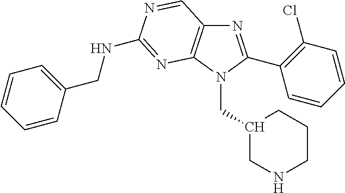
219 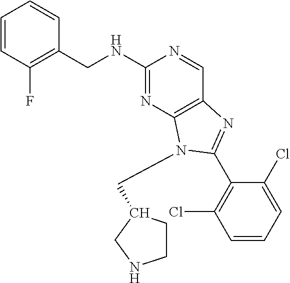
220 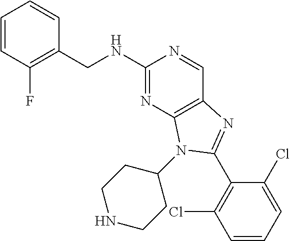
221 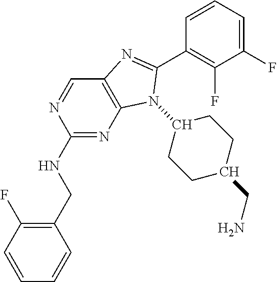
222 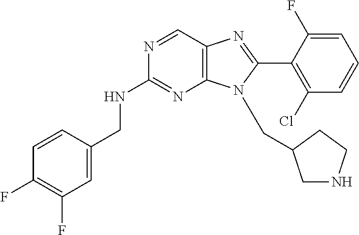
223 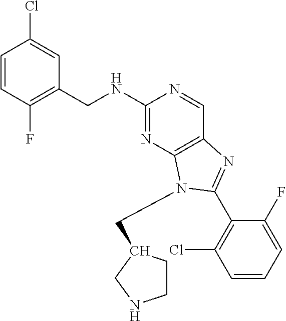
224 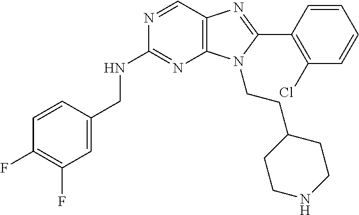
225 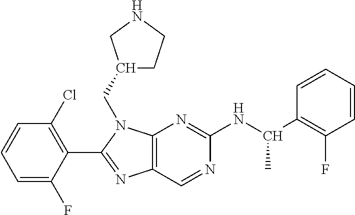
226 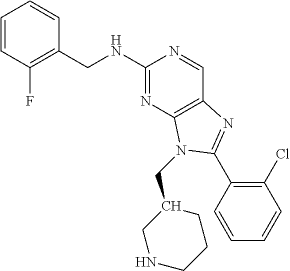
227 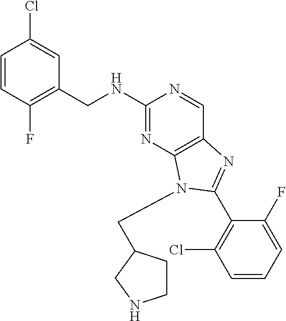
228 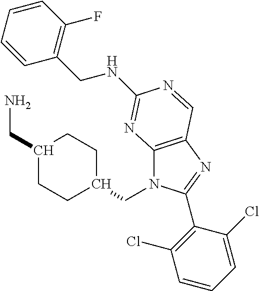
229 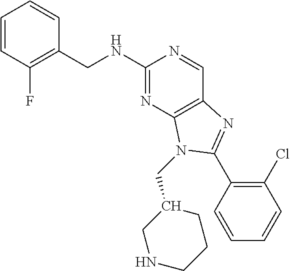
230 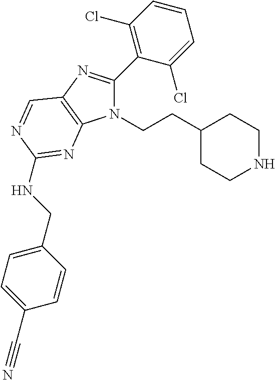
231 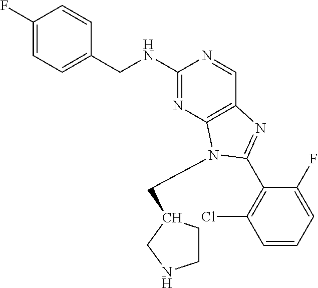
232 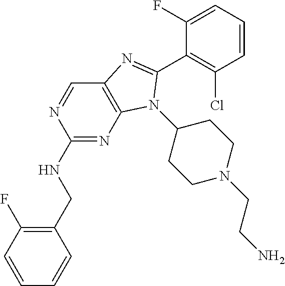
233 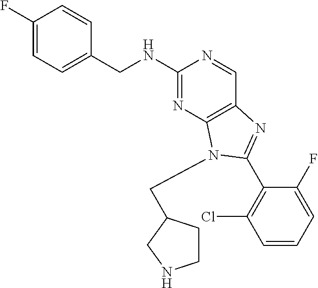
234 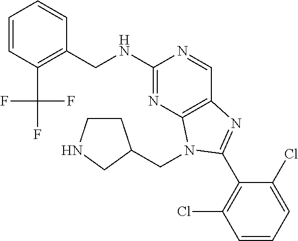
235 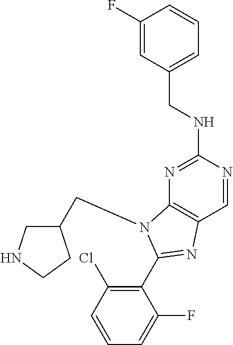
236 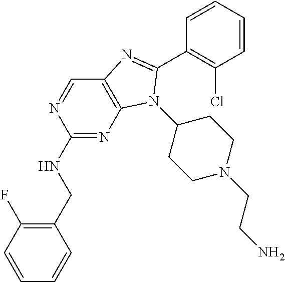
237 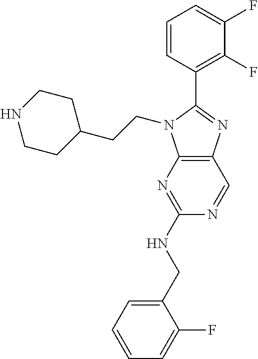
238 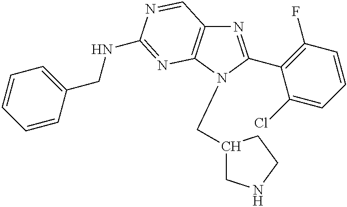
239 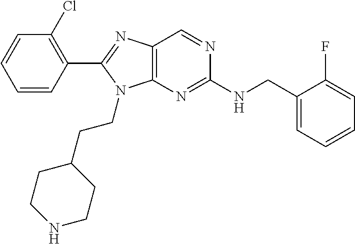
240 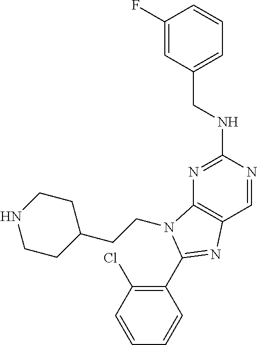
241 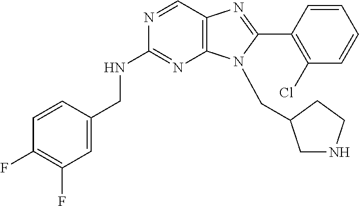
242 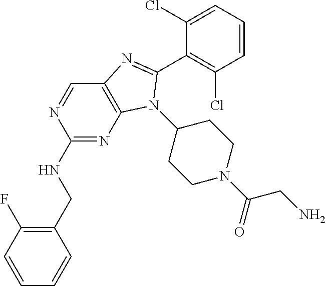
243 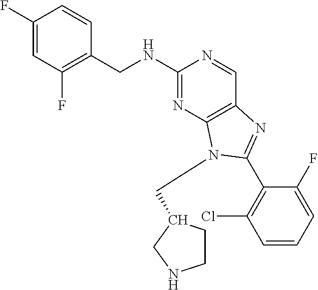
244 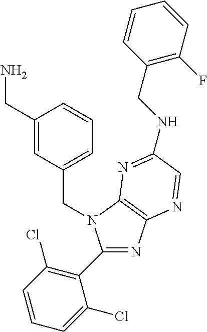
245 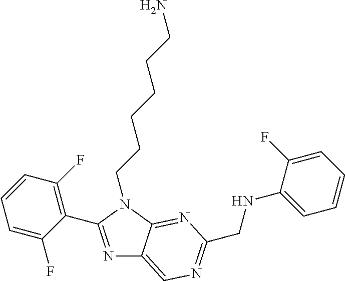
246 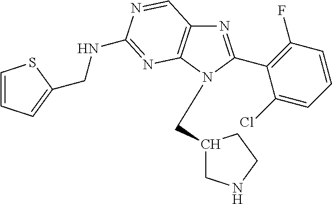
247 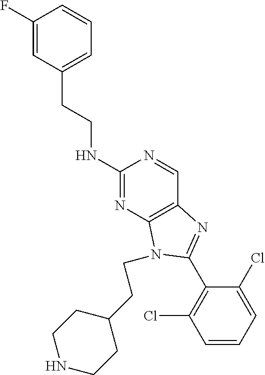
248 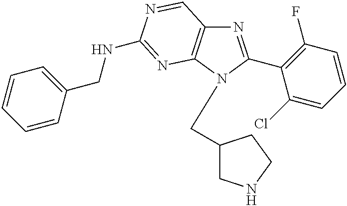
249 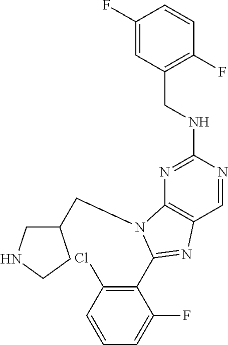
250 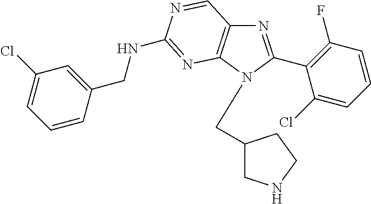
251 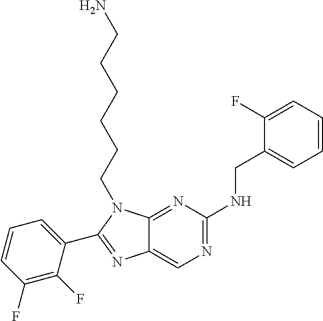
252 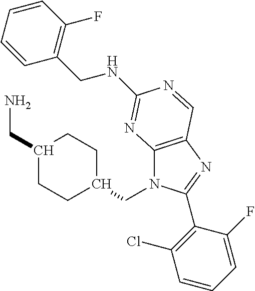
253 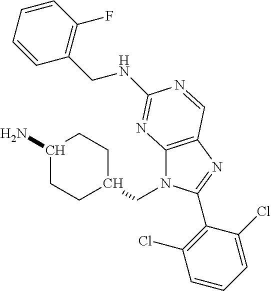
254 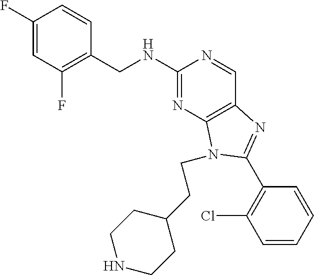
255 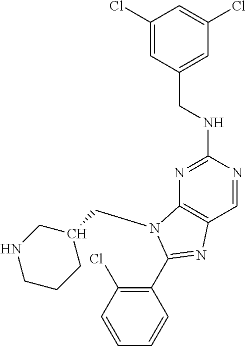
256 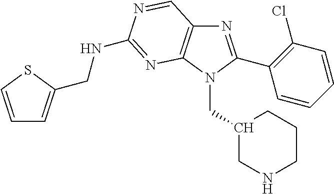
257 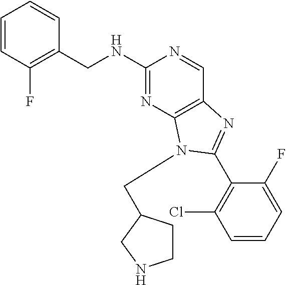
258 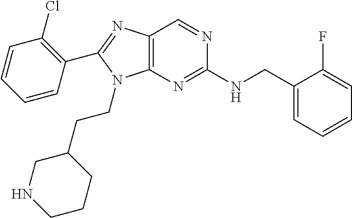
259 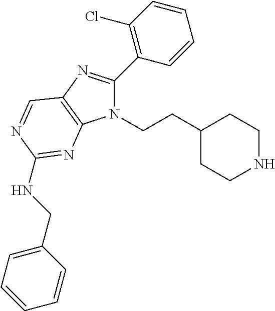
260 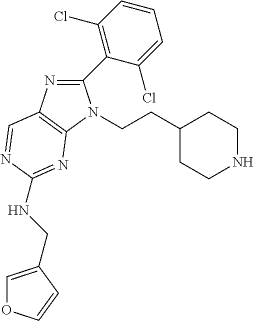
261 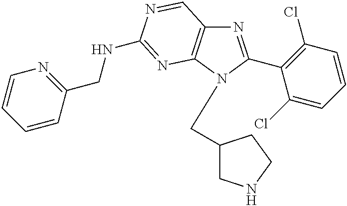
262 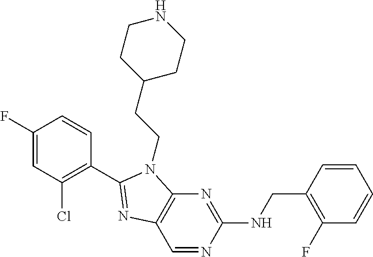
263 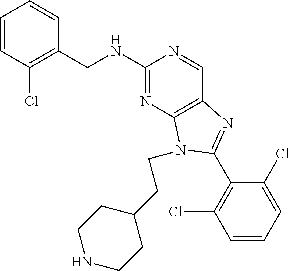
264 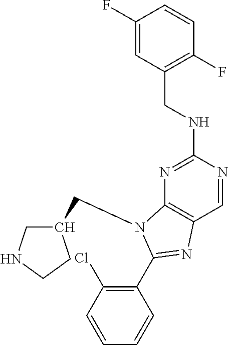
265 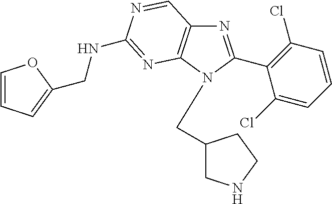
266 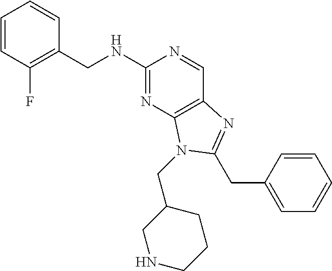
267 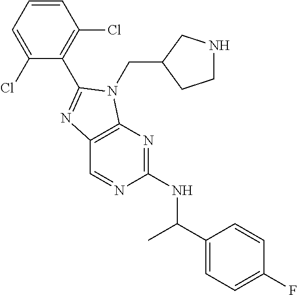
268 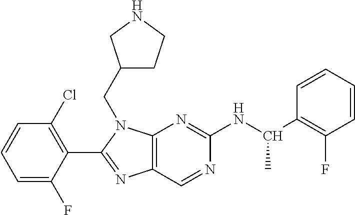
269 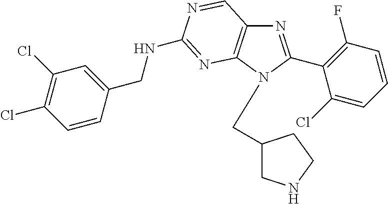
270 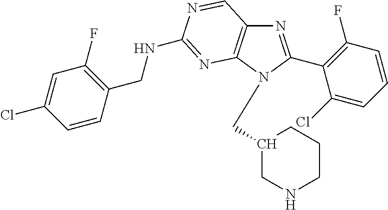
271 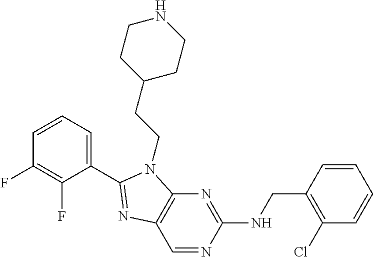
272 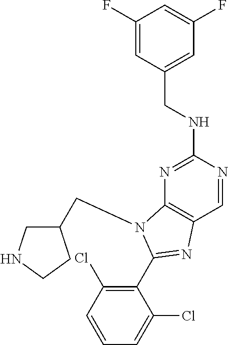
273 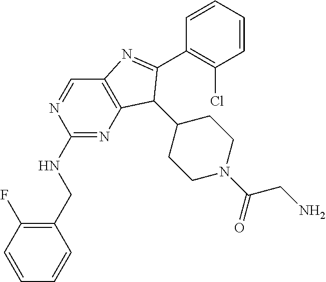
274 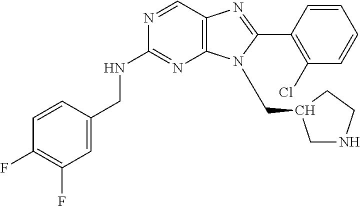
275 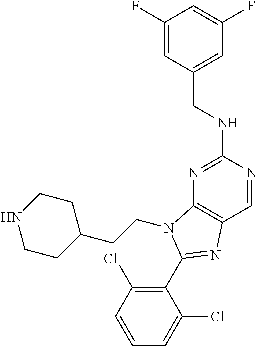
276 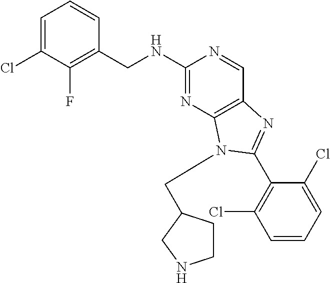
277 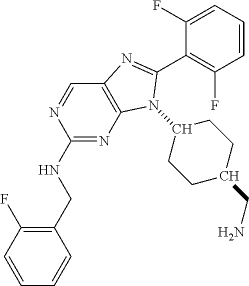
278 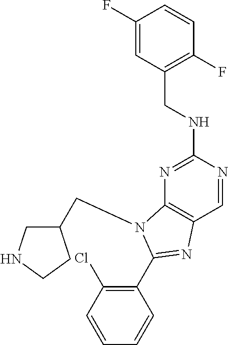
279 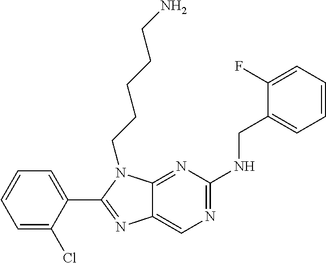
280 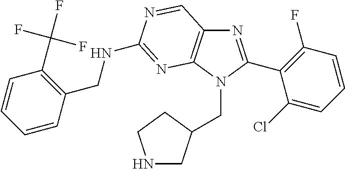
281 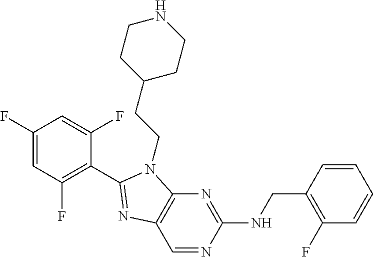
282 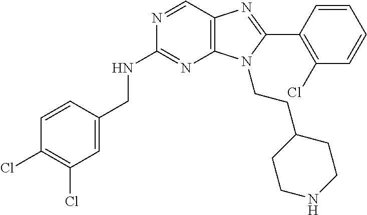
283 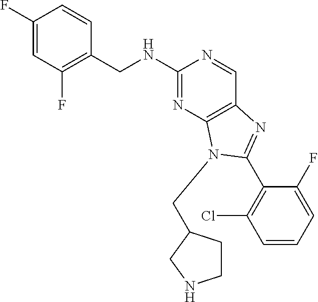
284 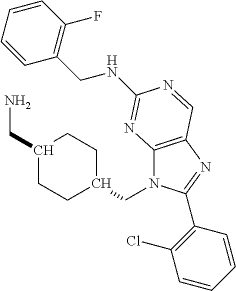
285 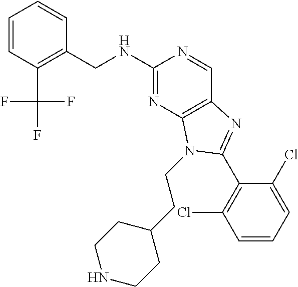
286 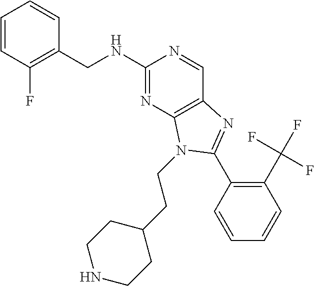
287 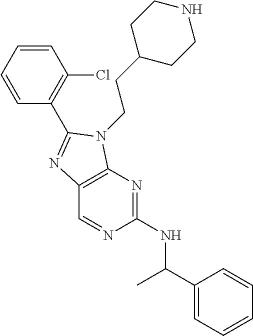
288 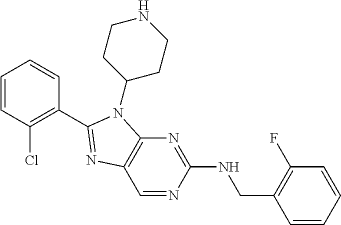
289 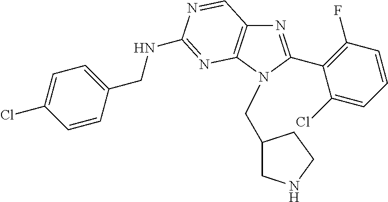
290 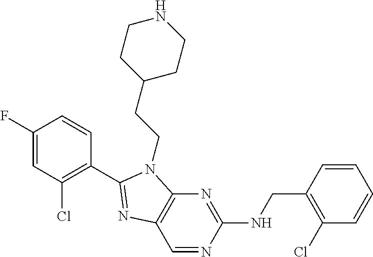
291 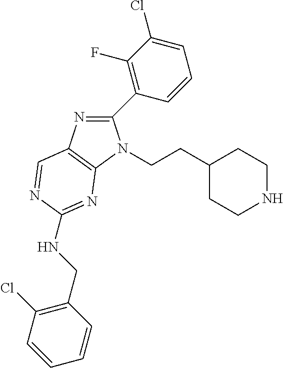
292 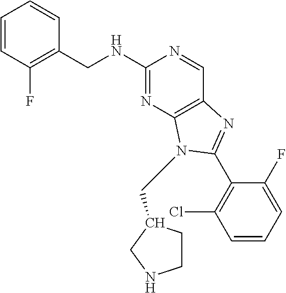
293 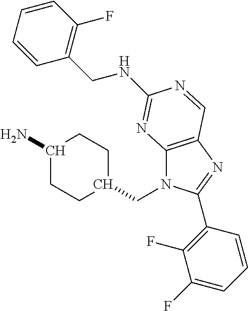
294 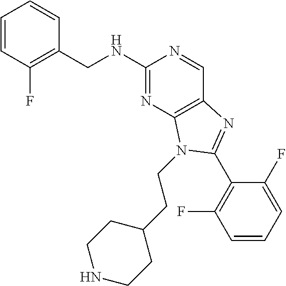
295 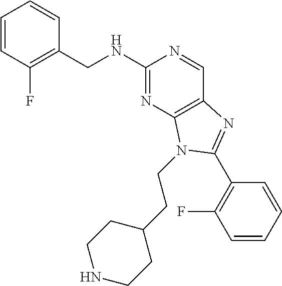
296 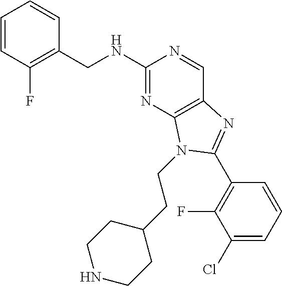
297 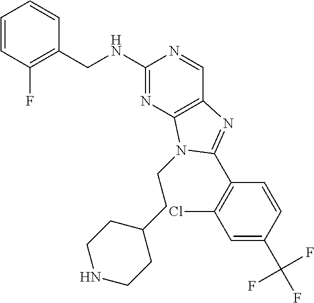
298 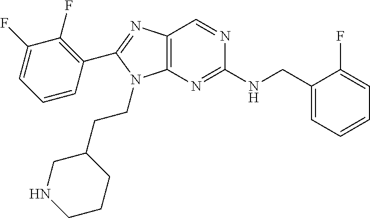
299 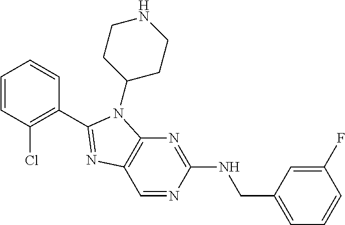
300 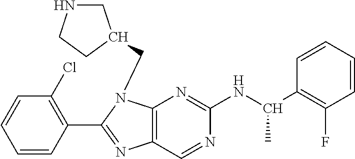
301 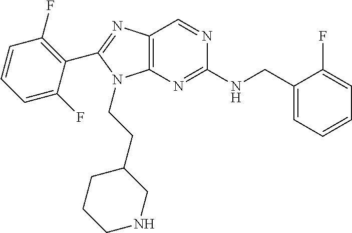
302 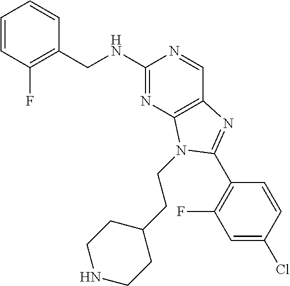
303 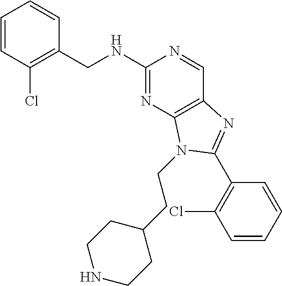
304 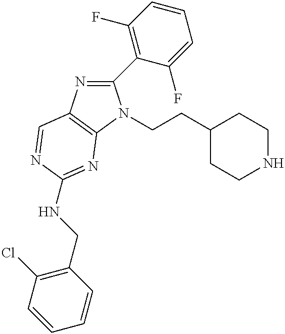
305 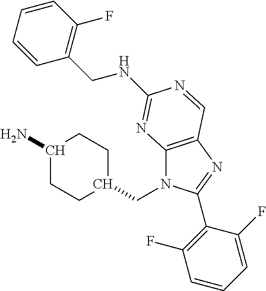
306 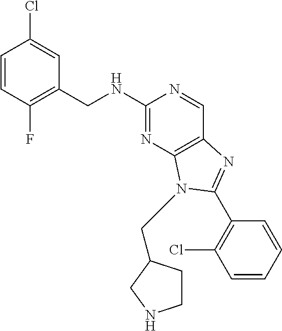
307 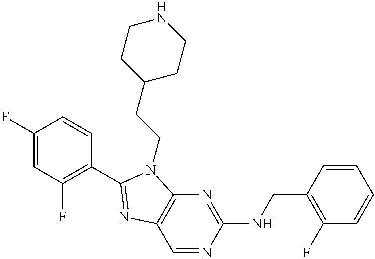
308 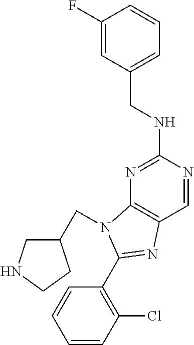
309 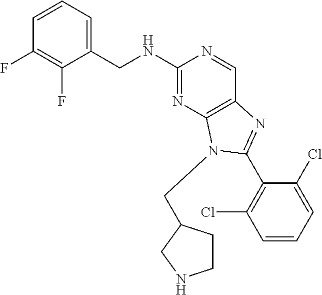
310 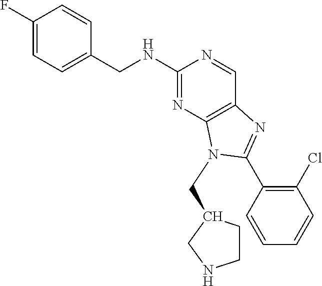
311 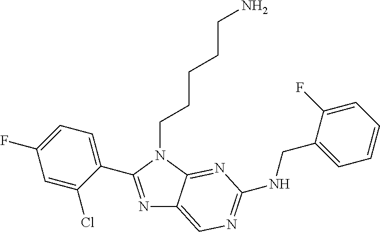
312 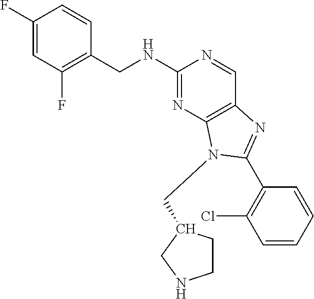
313 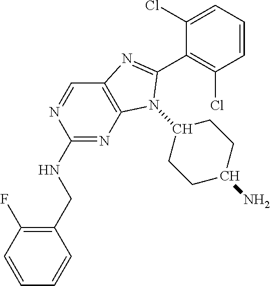
314 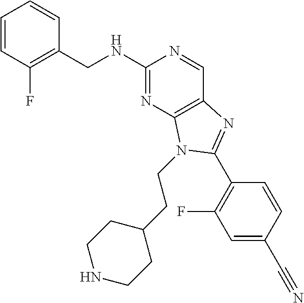
315 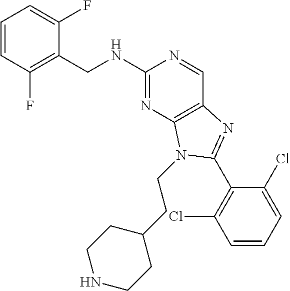
316 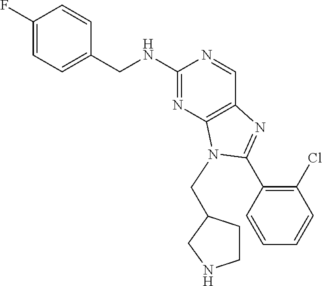
317 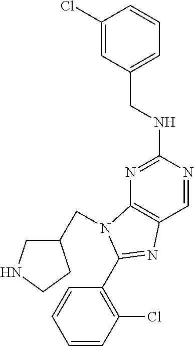
318 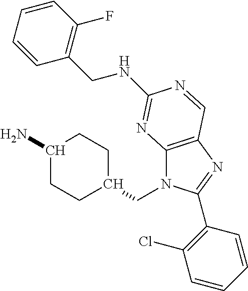
319 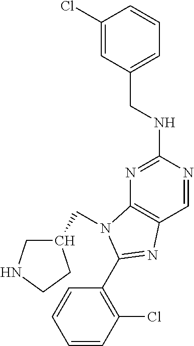
320 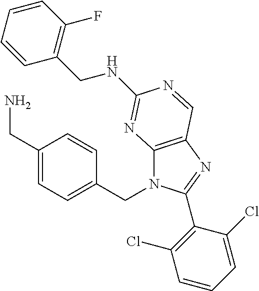
321 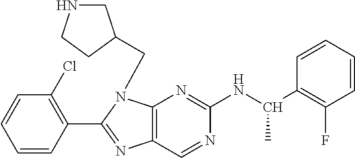
322 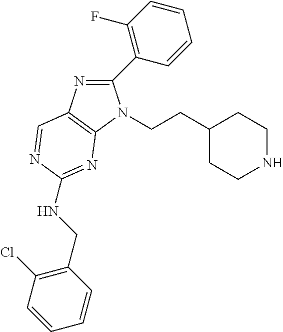
323 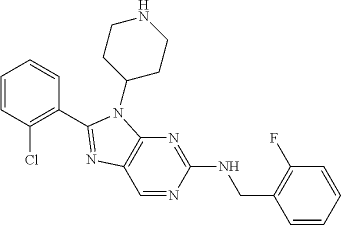
324 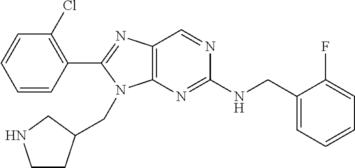
325 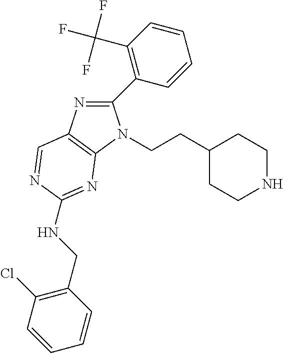
326 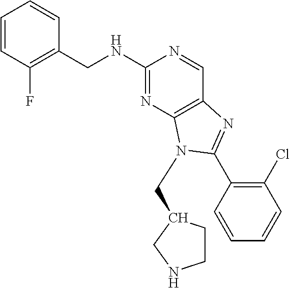
327 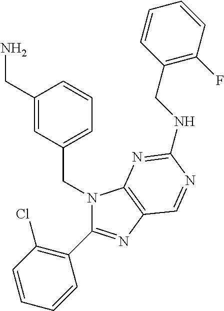
328 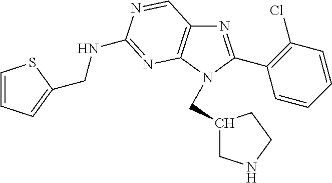
329 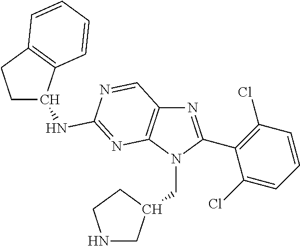
330 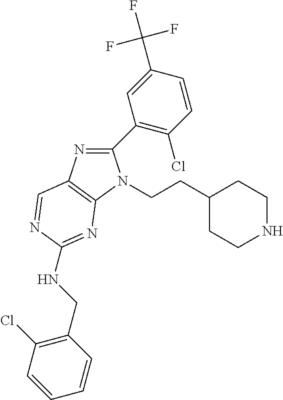
331 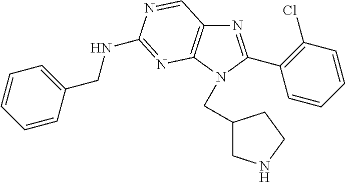
400 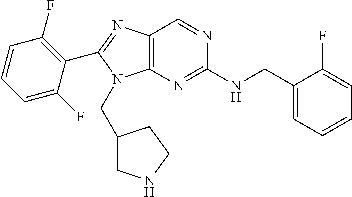
401 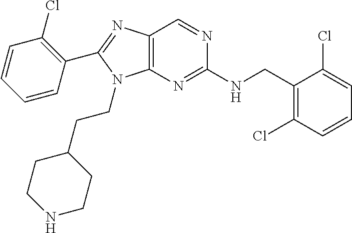
402 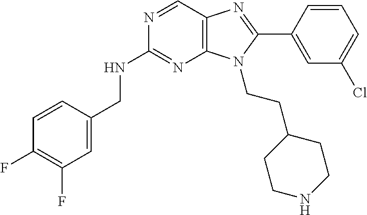
403 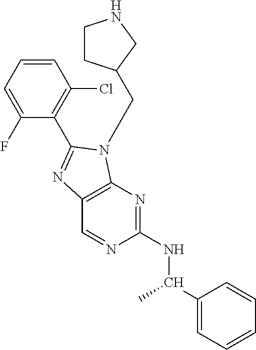
404 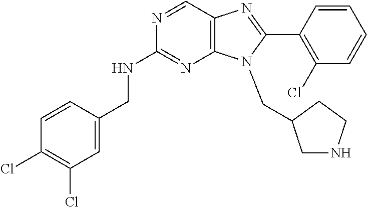
405 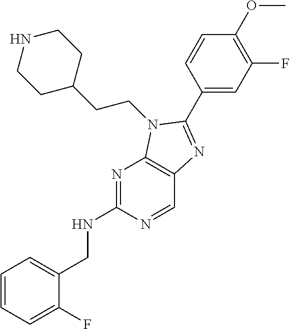
406 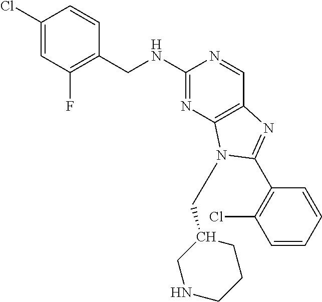
407 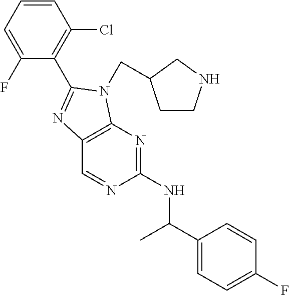
408 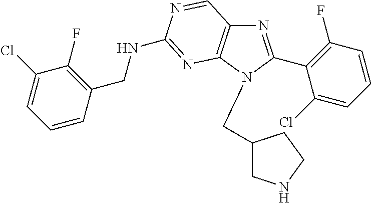
409 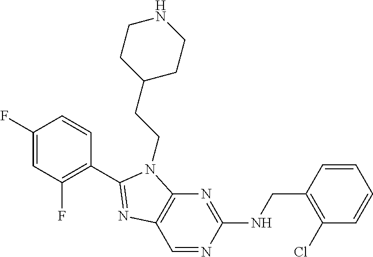
410 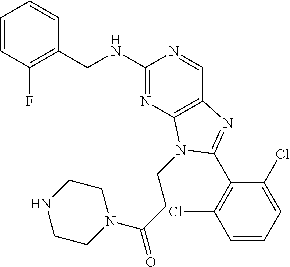
411 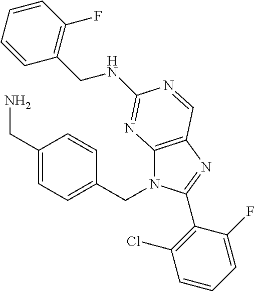
412 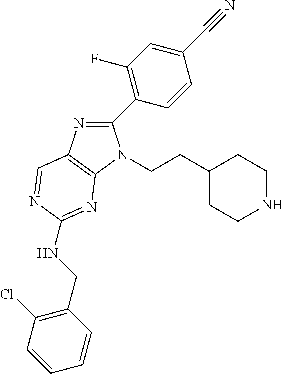
413 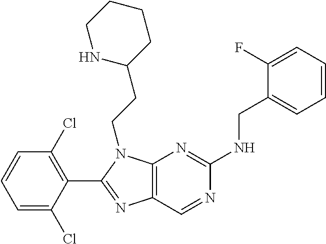
414 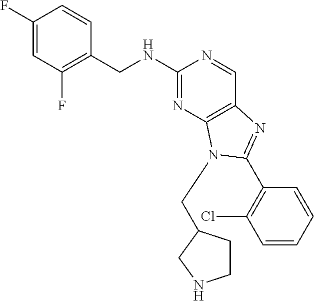
415 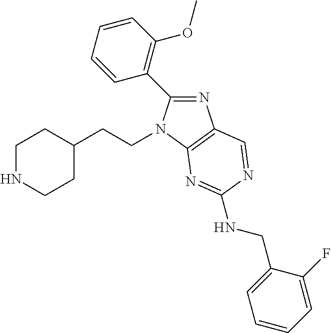
416 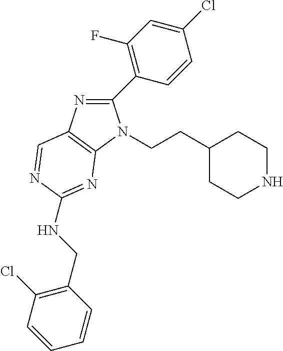
417 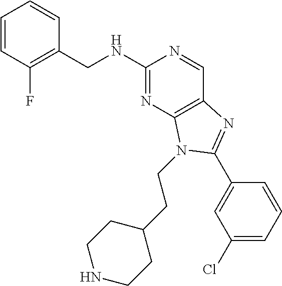
418 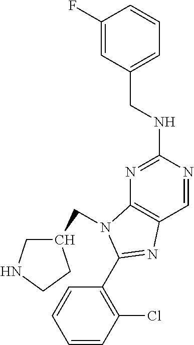
419 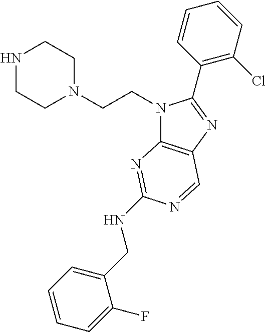
420 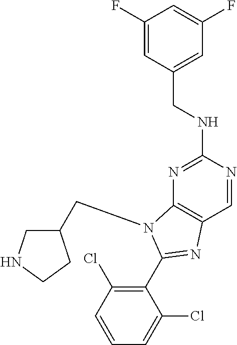
421 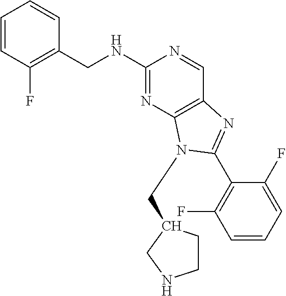
422 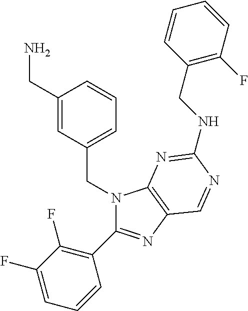
423 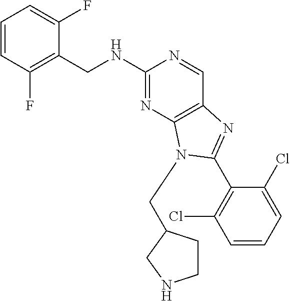
424 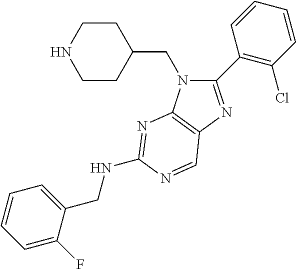
425 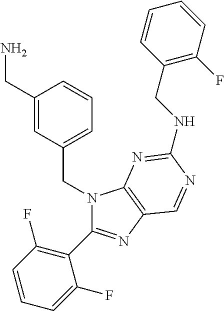
426 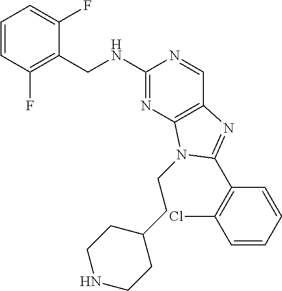
427 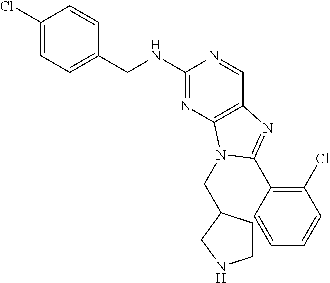
428 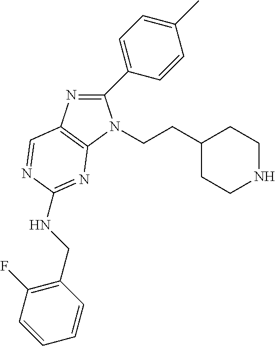
429 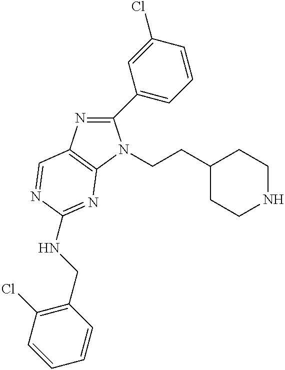
430 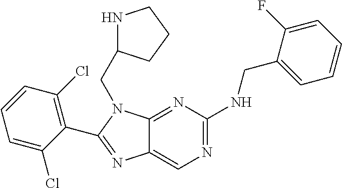
431 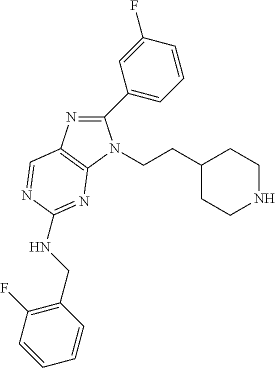
432 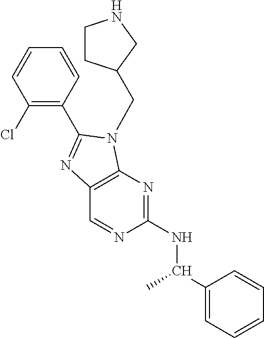
433 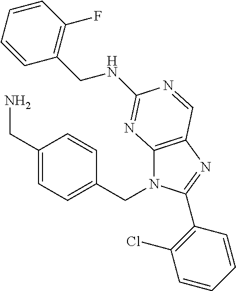
434 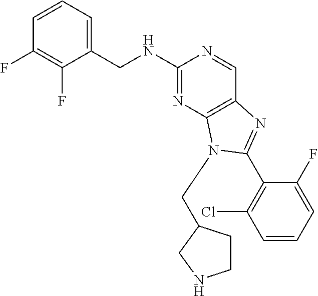
435 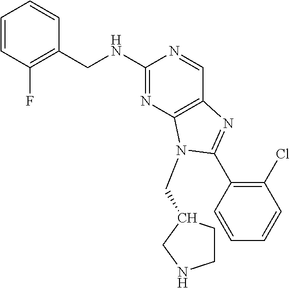
436 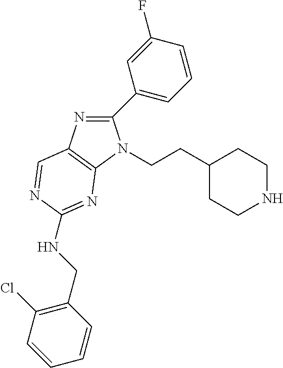
437 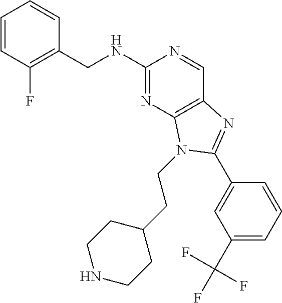
438 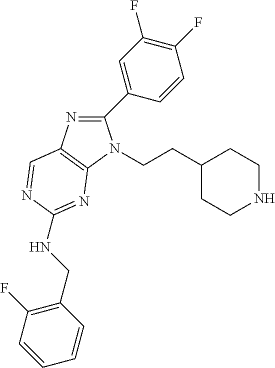
439 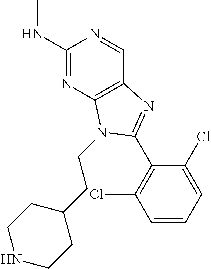
440 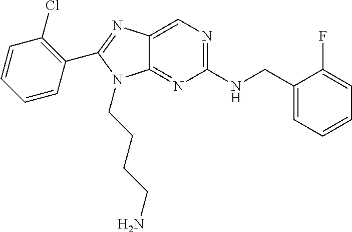
441 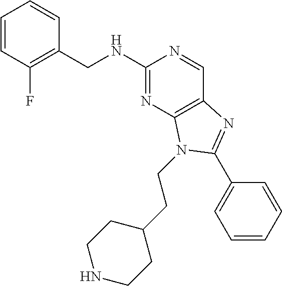
442 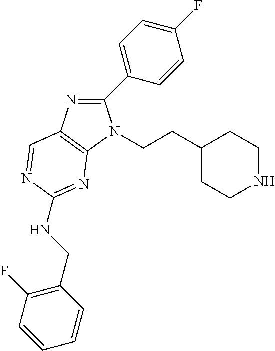
443 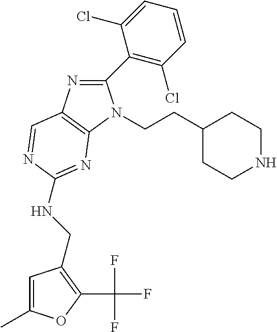
444 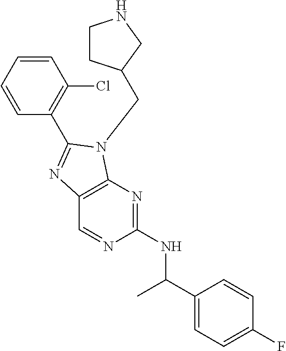
445 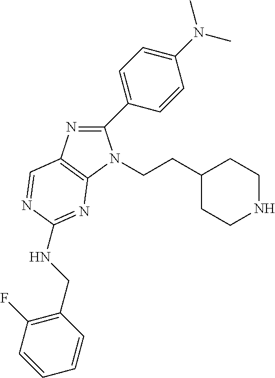
446 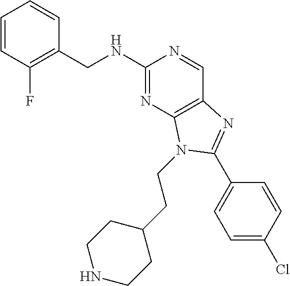
447 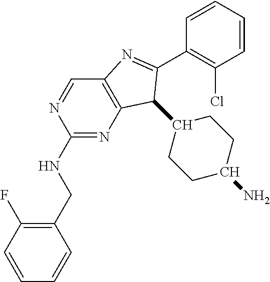
448 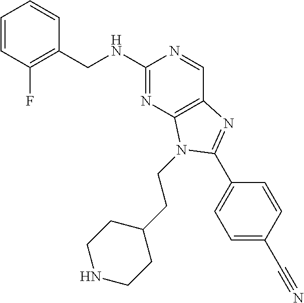
449 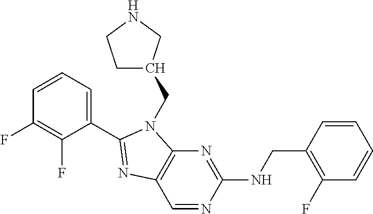
450 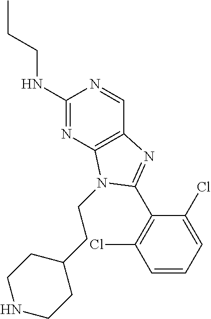
451 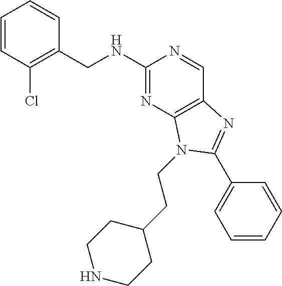
452 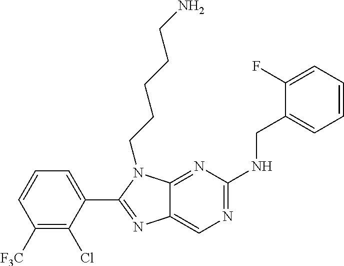
453 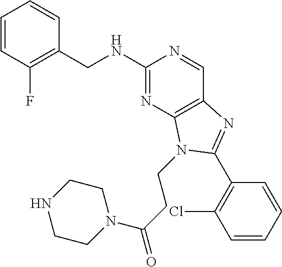
454 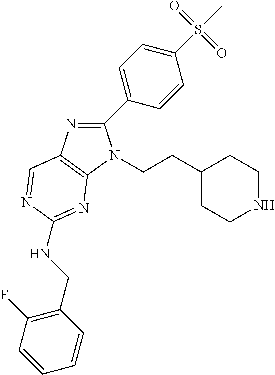
455 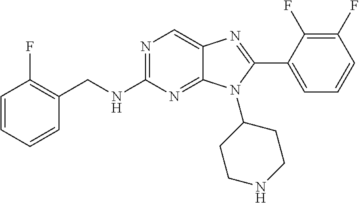
456 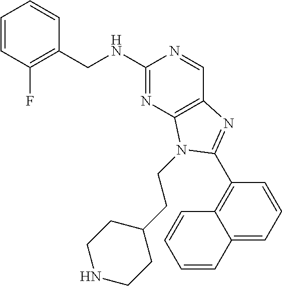
457 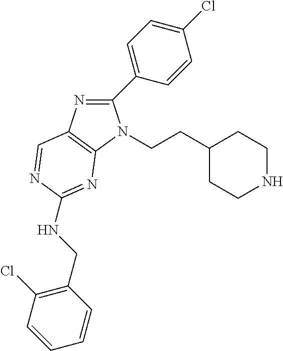
458 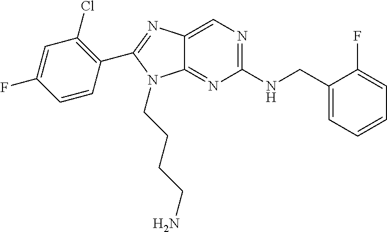
459 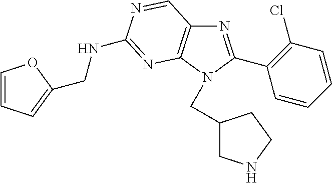
460 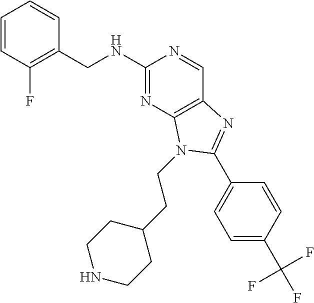
461 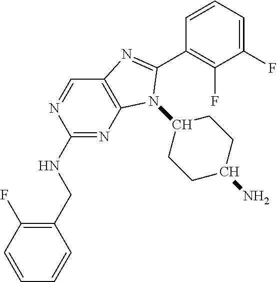
462 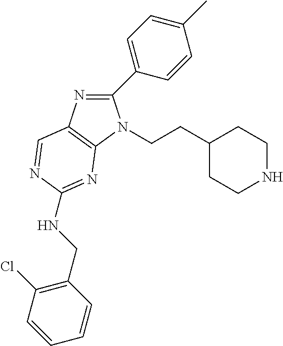
463 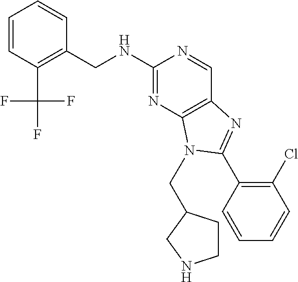
464 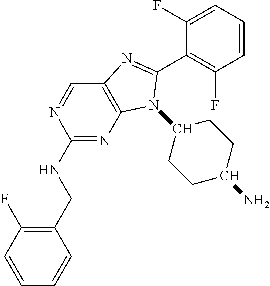
465 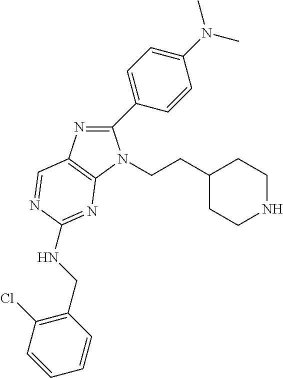
466 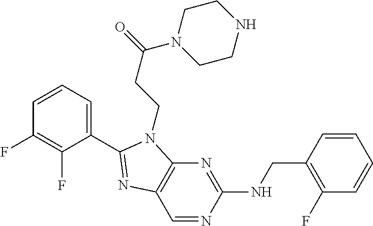
467 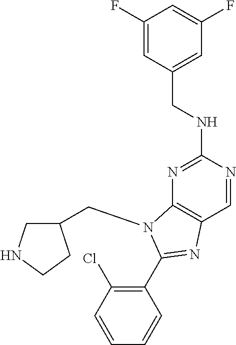
468 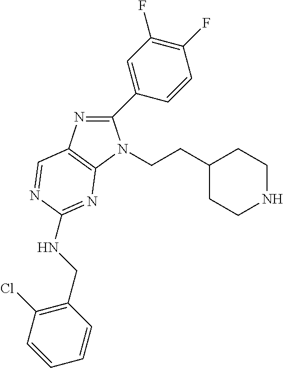
469 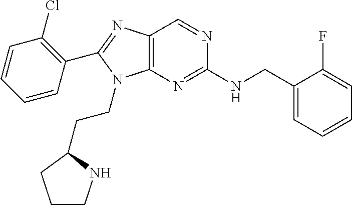
470 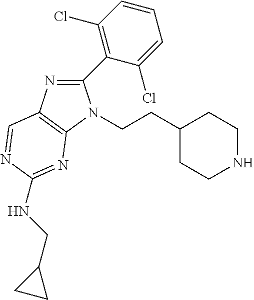
471 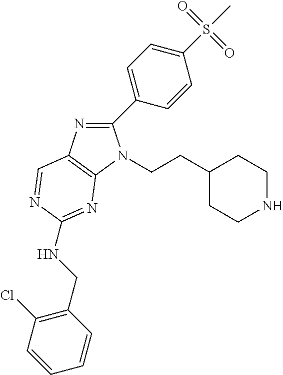
472 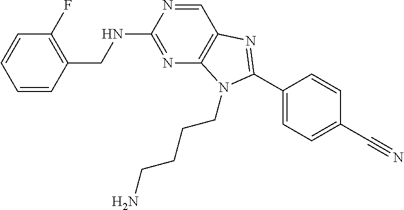
473 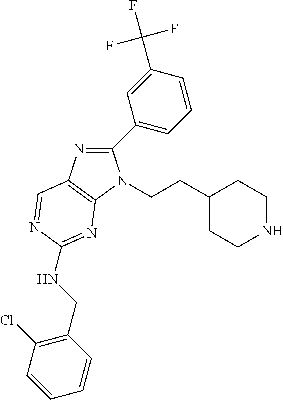
474 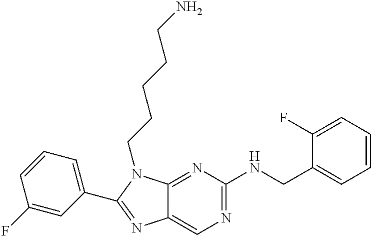
475 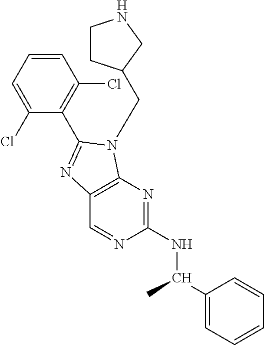
476 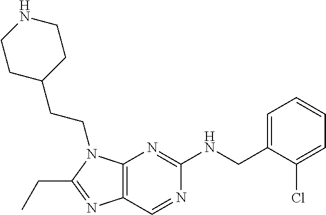
477 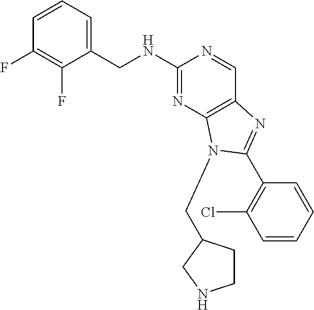
478 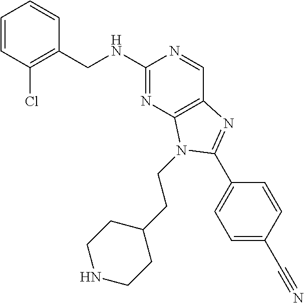
479 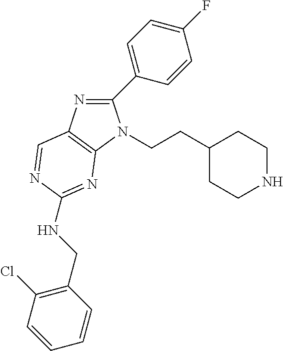
480 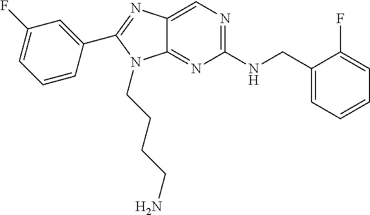
481 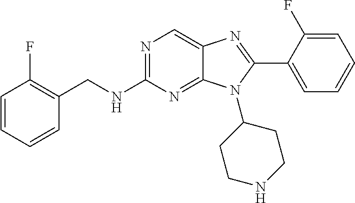
482 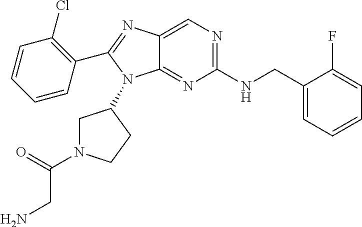
483 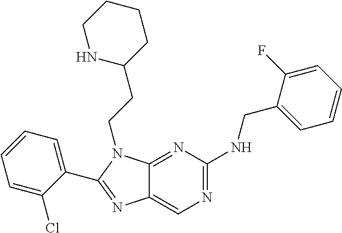
484 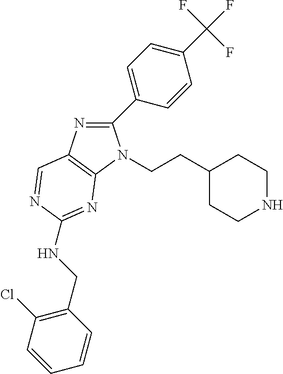
485 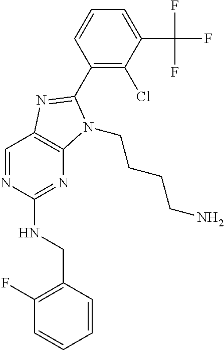
486 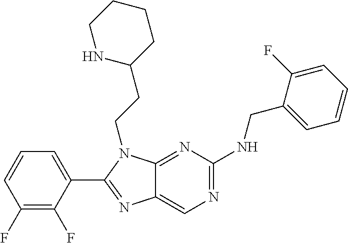
487 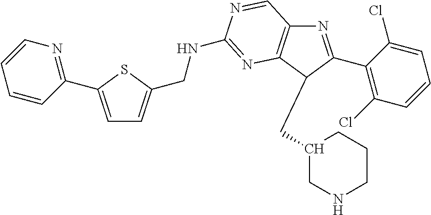
488 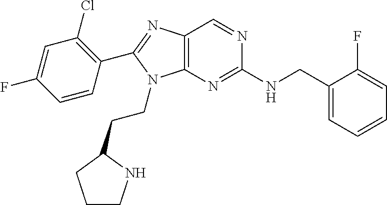
489 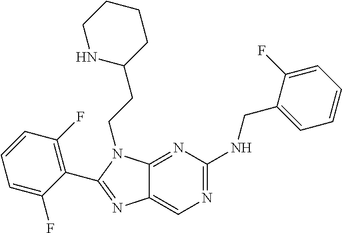
490 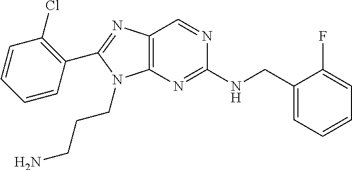
491 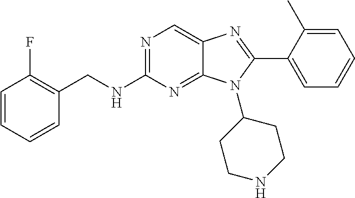
492 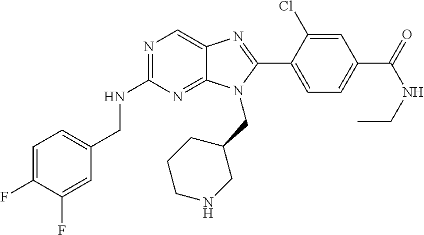
500 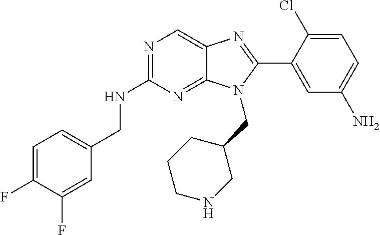
501 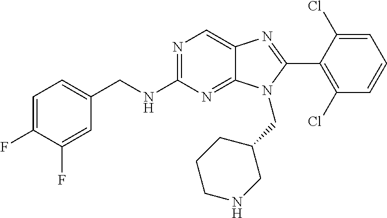
502 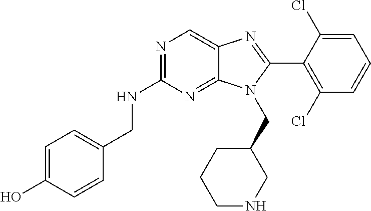
503 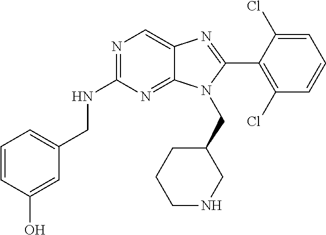
504 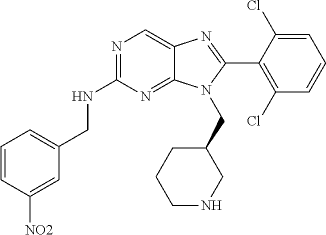
505 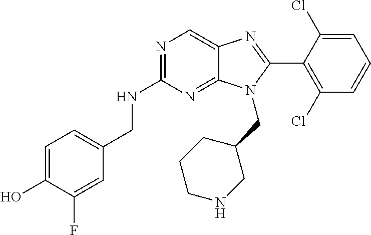
506 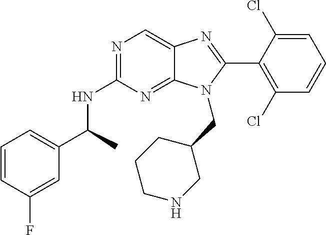
507 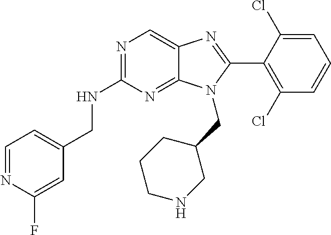
508 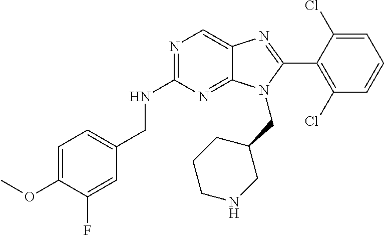
509 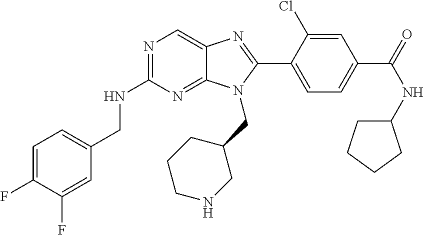
510 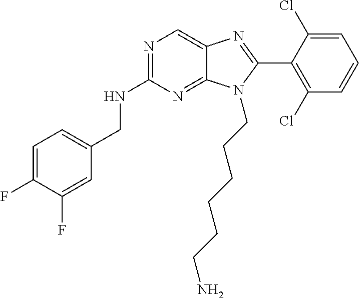
511 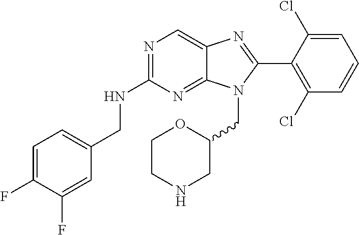
512 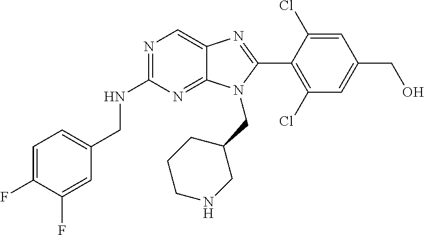
513 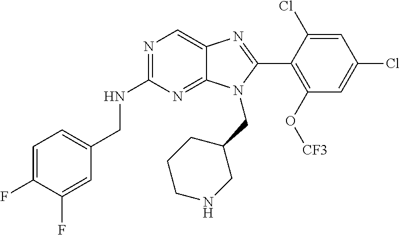
514 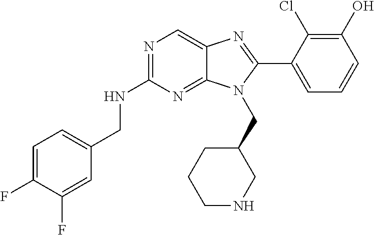
515 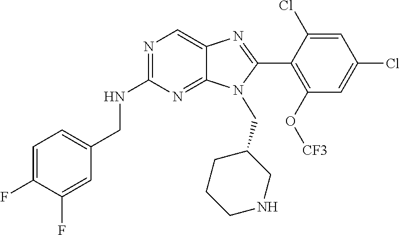
516 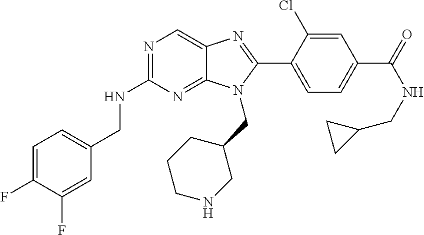
517 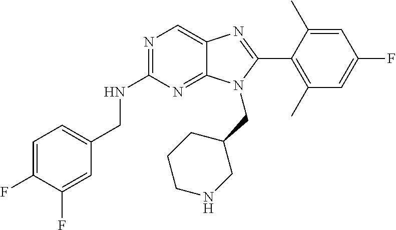
518 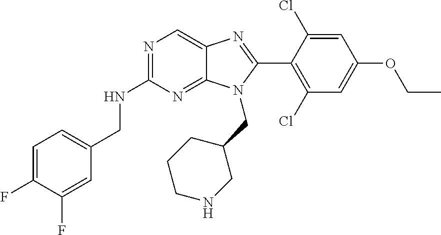
519 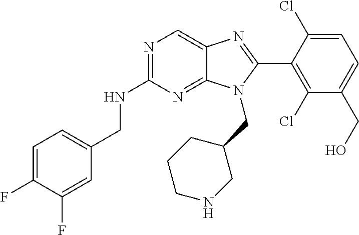
520 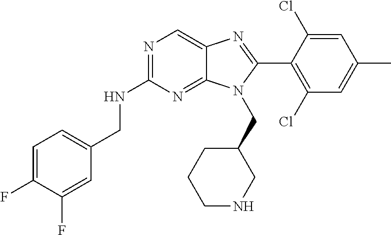
521 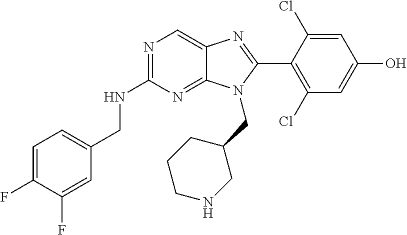
522 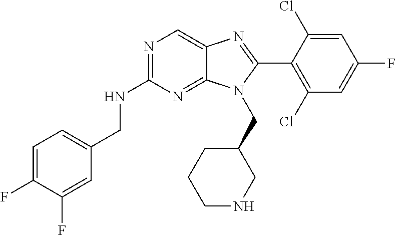
523 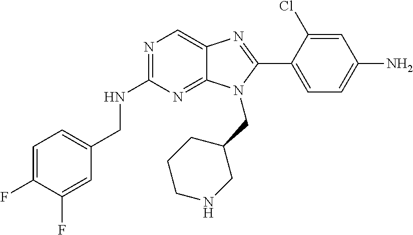
524 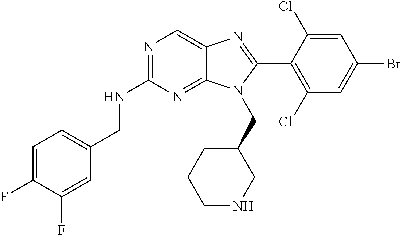
525 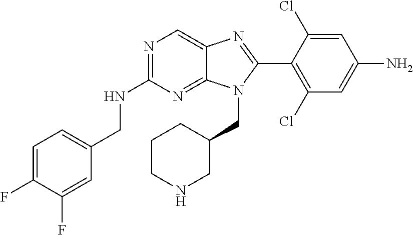
526 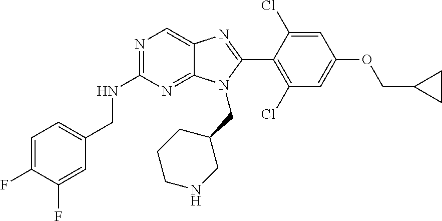
527 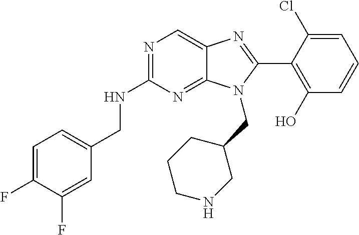
528 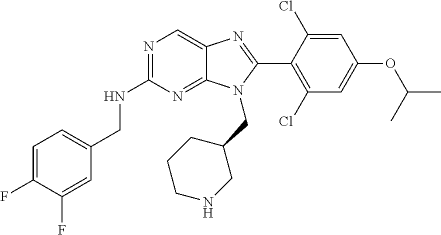
529 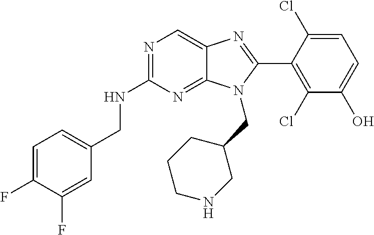
530 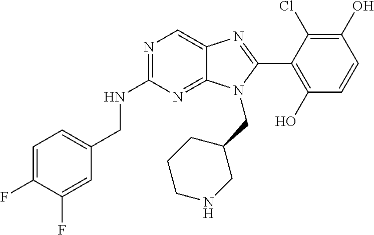
531 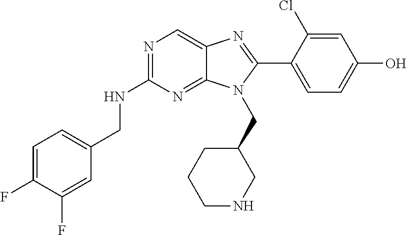
532 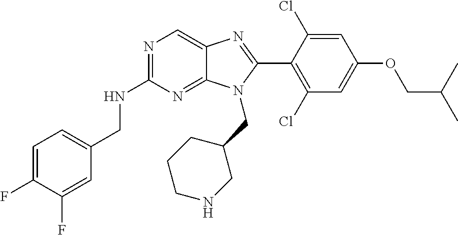
533 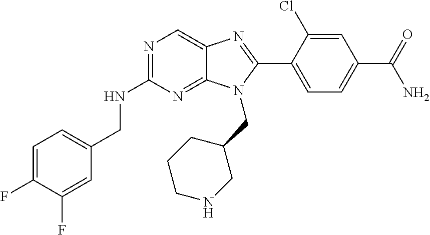
534 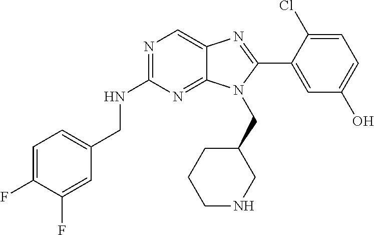
535 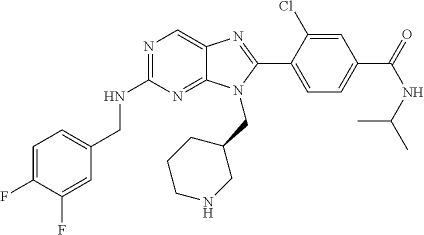
536 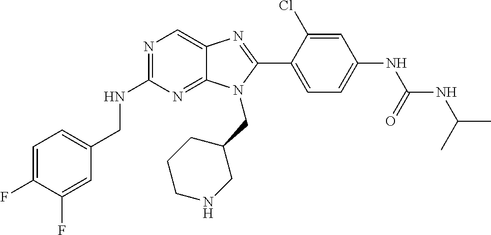
537 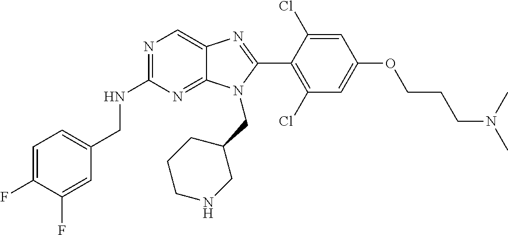
538 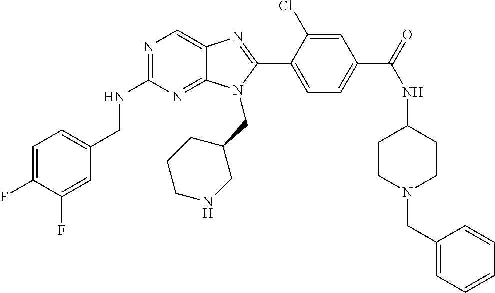
539 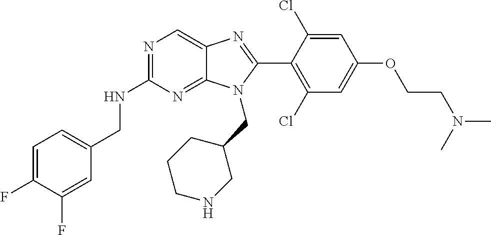
540 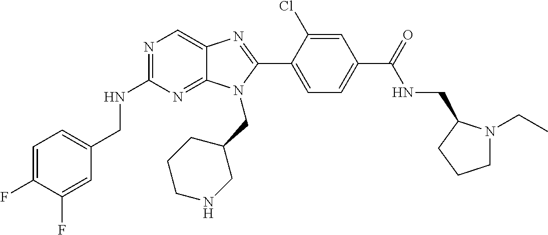
541 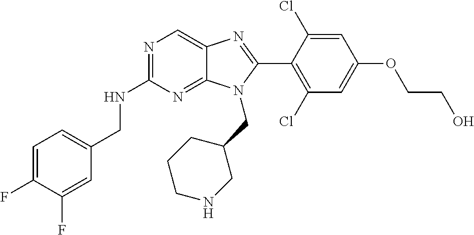
542 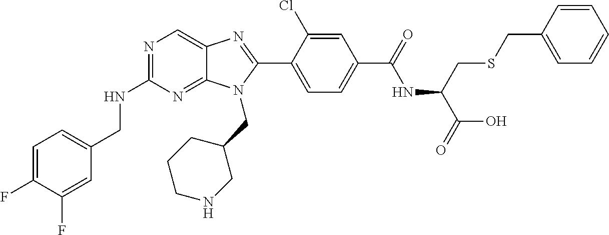
543 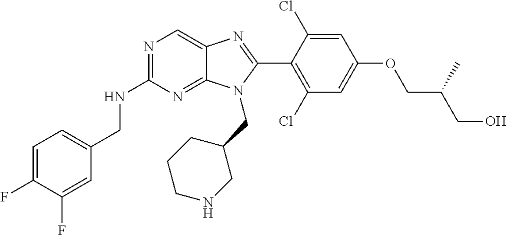
544 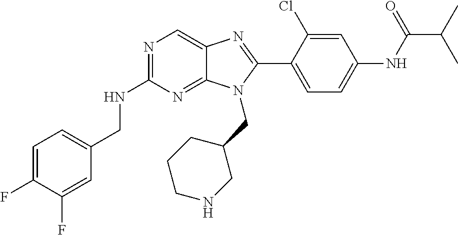
545 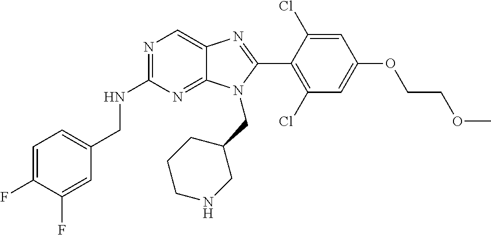
546 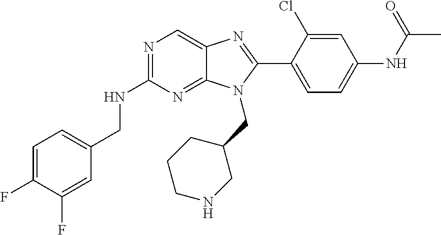
547 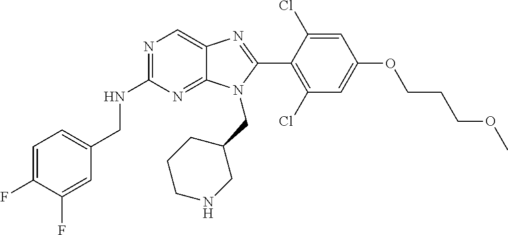
548 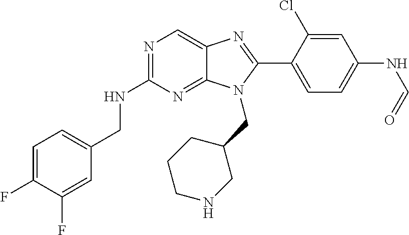
549 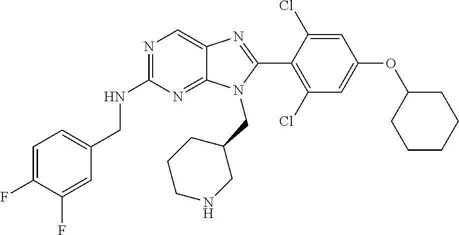
550 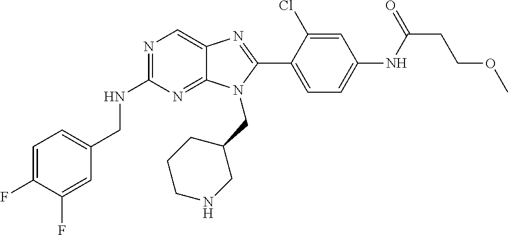
551 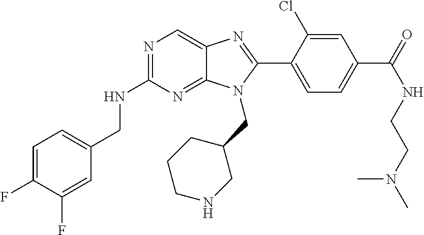
552 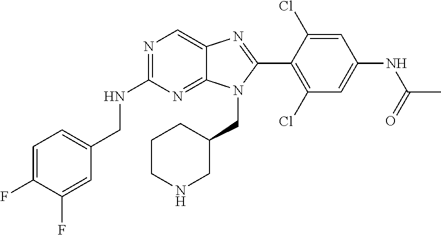
553 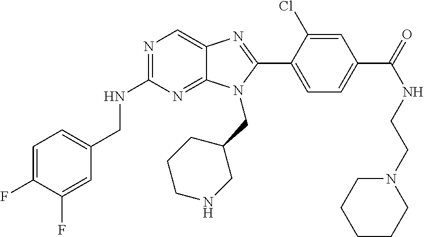
554 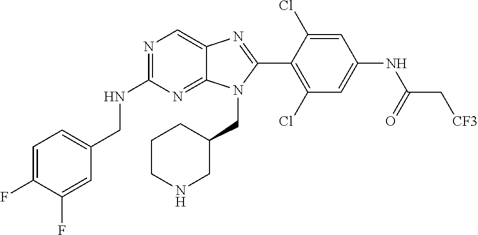
555 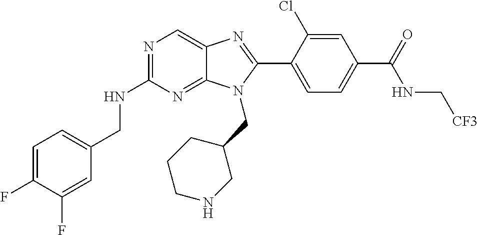
556 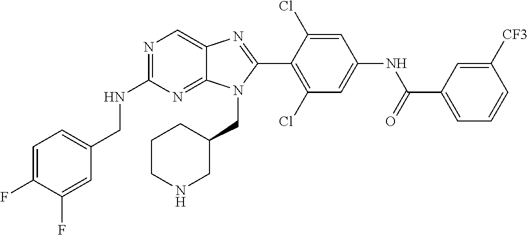
557 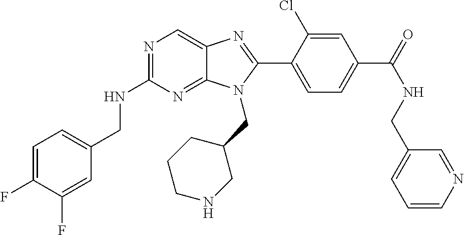
558 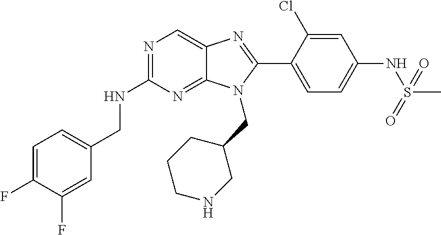
559 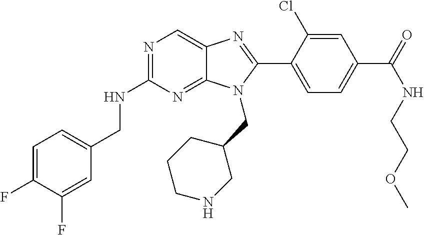
560 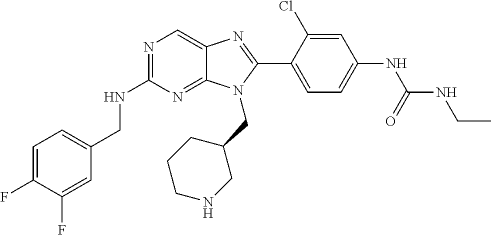
561 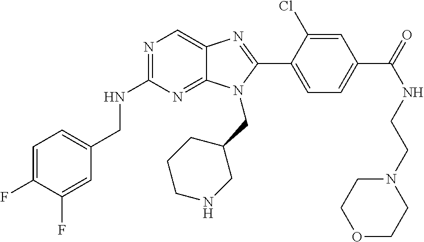
562 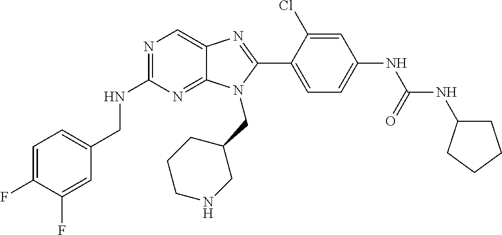
563 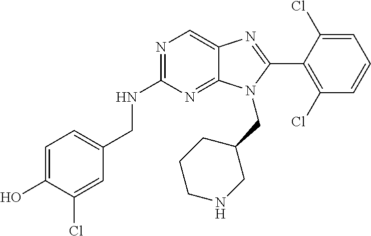
564 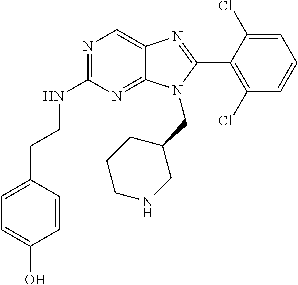
600 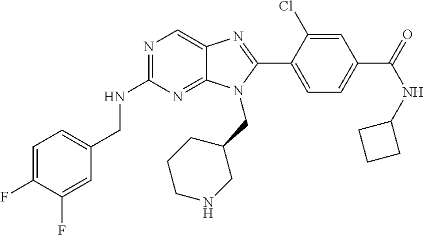
601 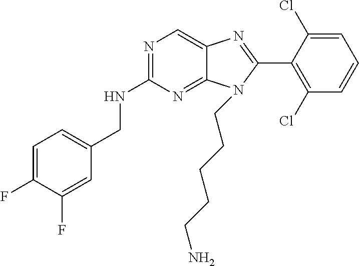
602 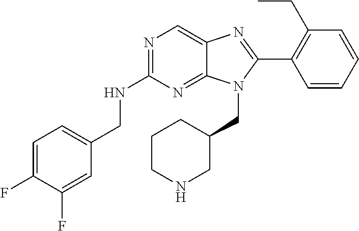
603 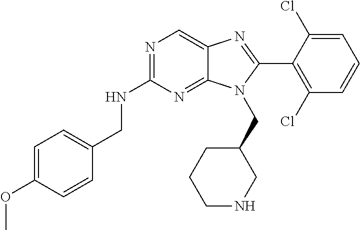
604 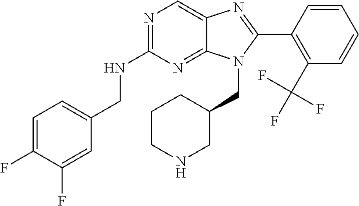
605 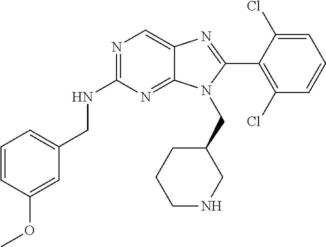
606 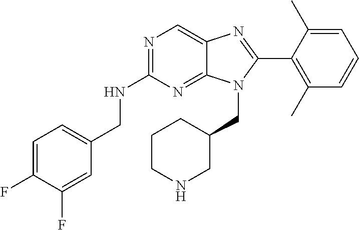
607 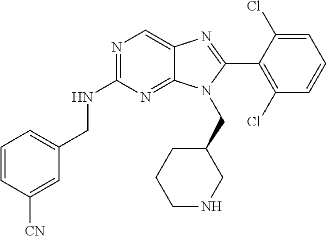
608 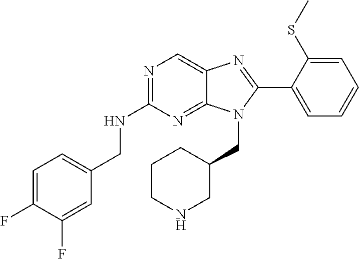
609 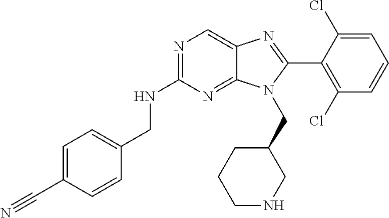
610 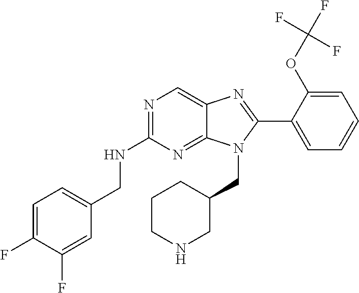
611 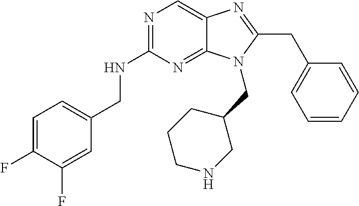
612 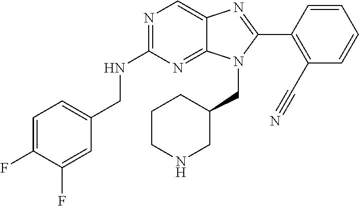
613 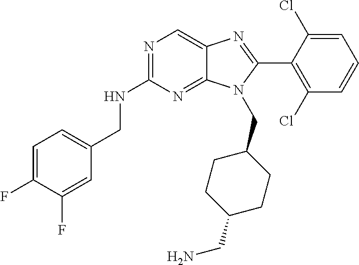
614 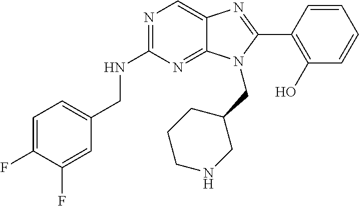
615 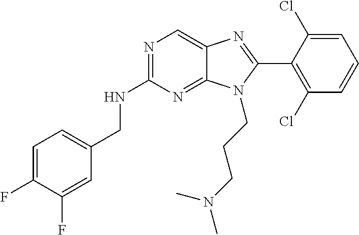
616 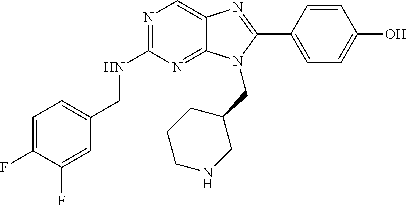
617 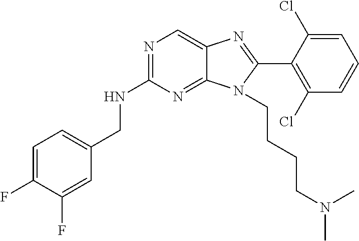
618 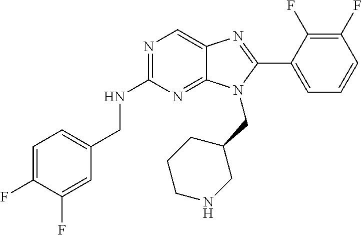
619 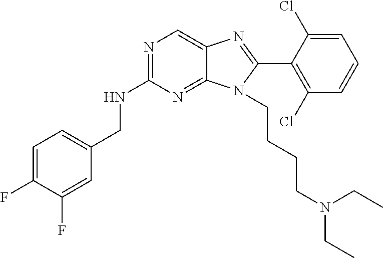
620 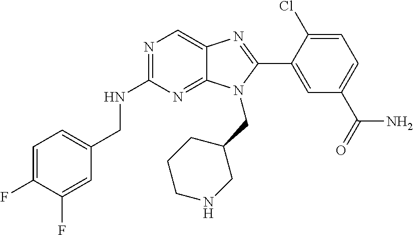
621 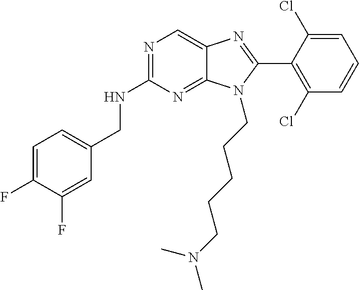
622 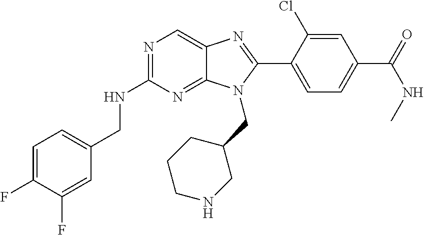
623 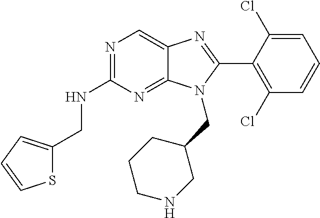
624 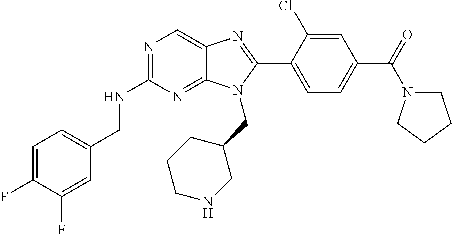
625 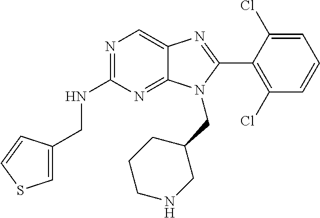
626 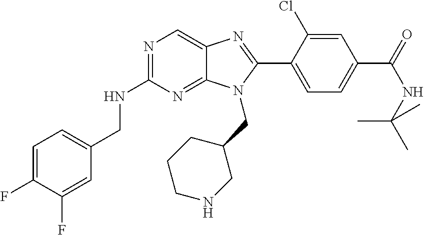
627 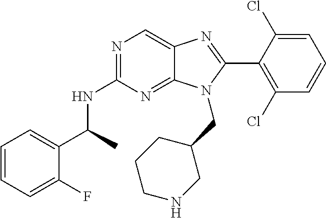
628 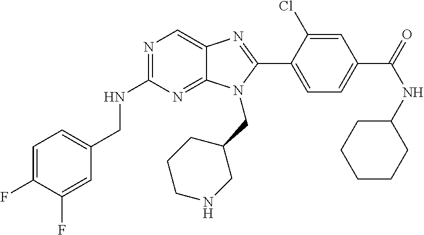
629 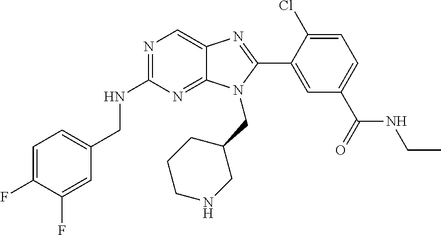
630 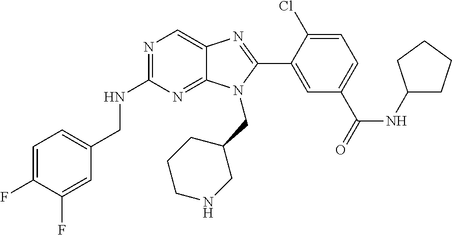
631 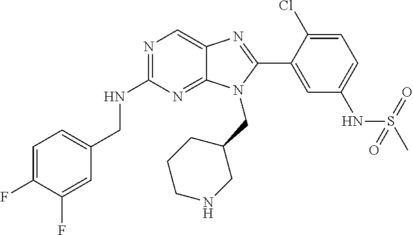
632 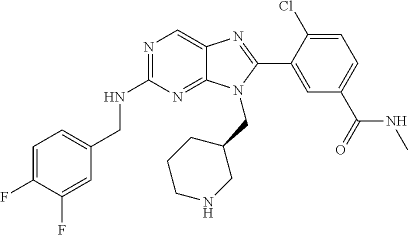
633 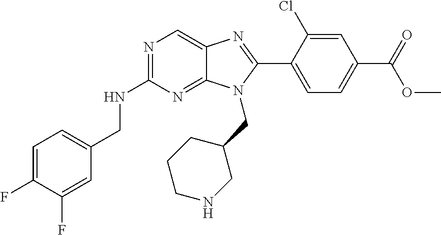
634 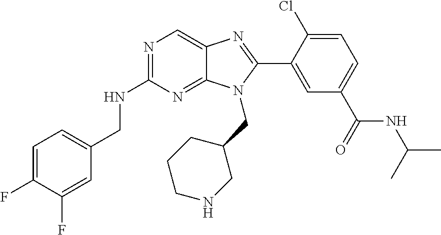
635 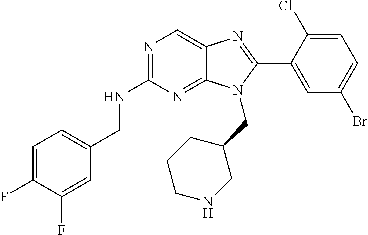
636 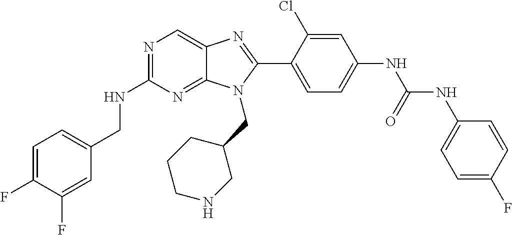
637 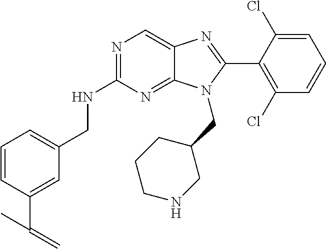
638 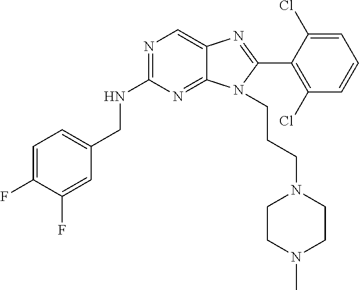
639 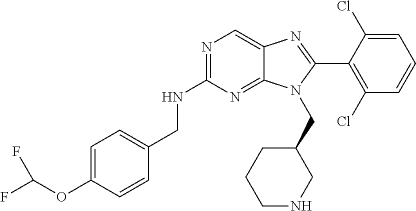
640 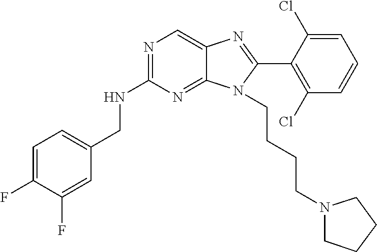
641 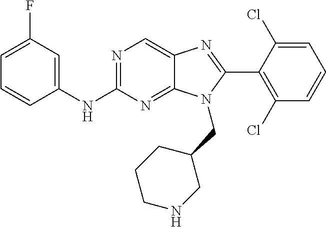
642 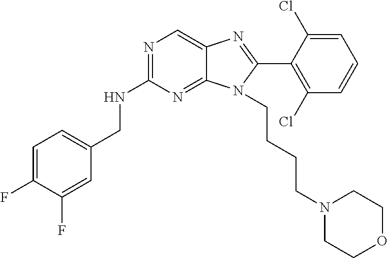
643 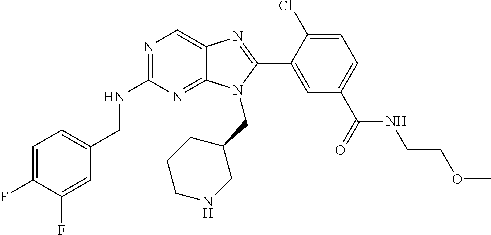
644 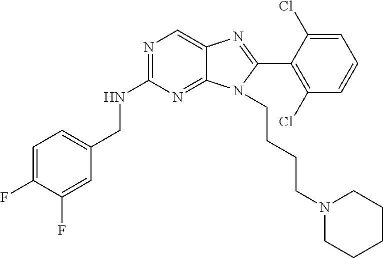
645 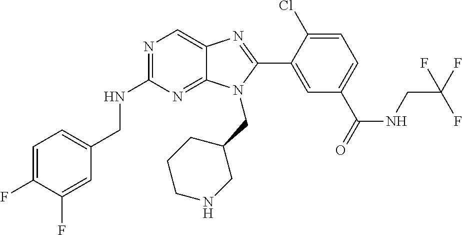
646 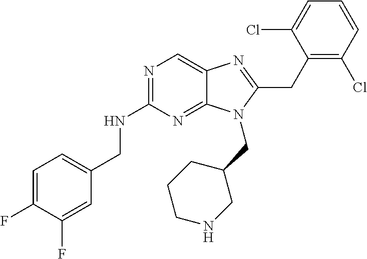
647 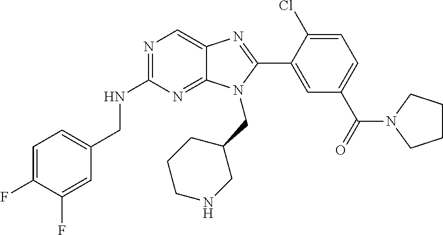
648 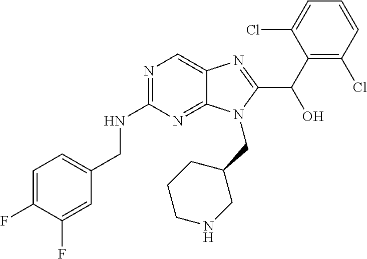
649 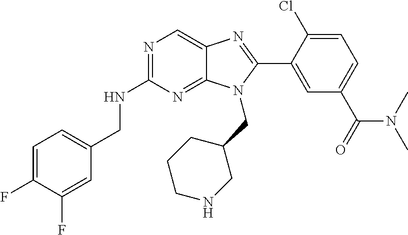
650 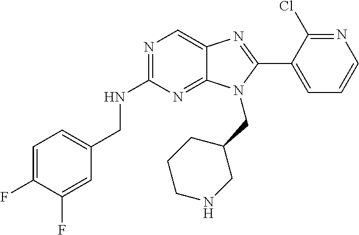
651 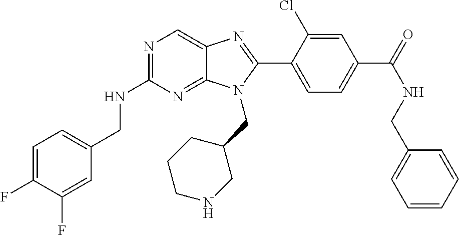
652 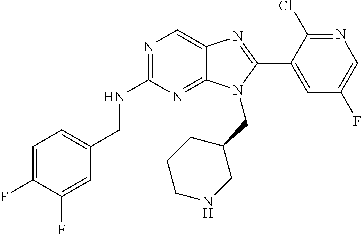
653 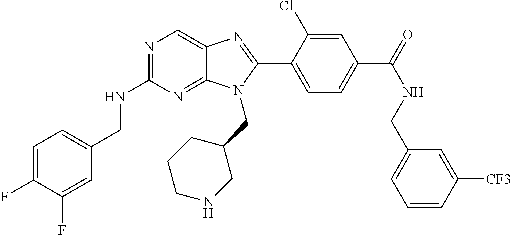
654 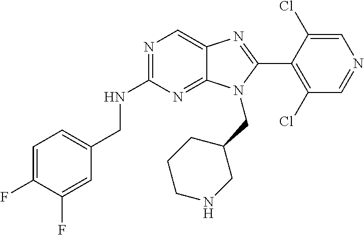
655 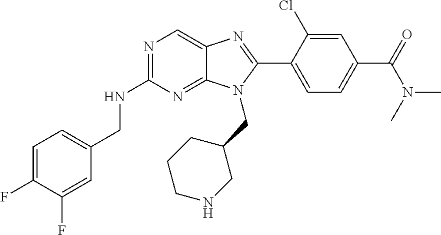
656 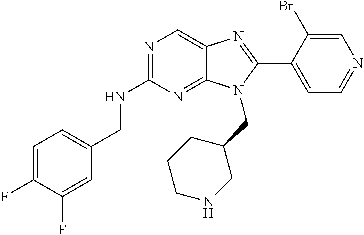
657 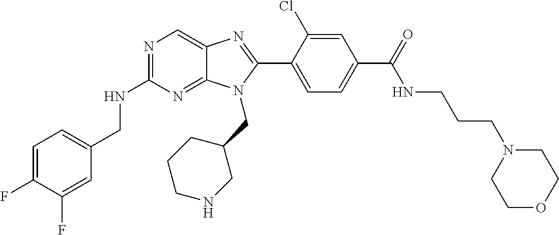
658 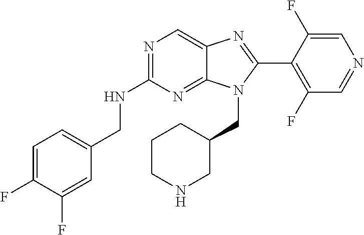
659 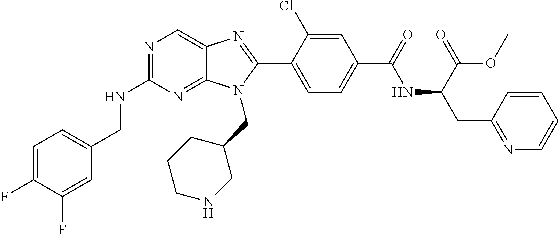
660 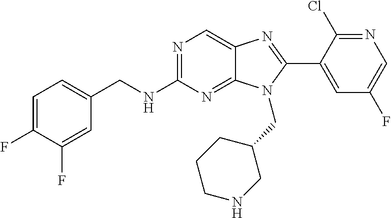
661 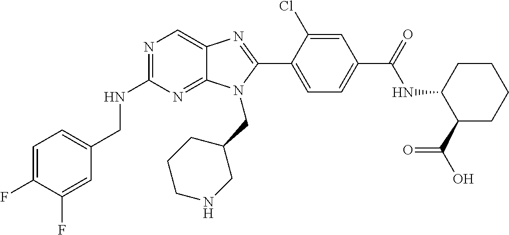
662 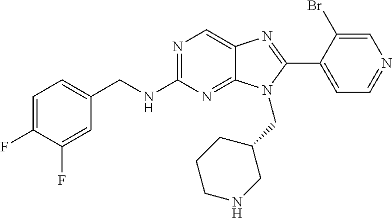
663 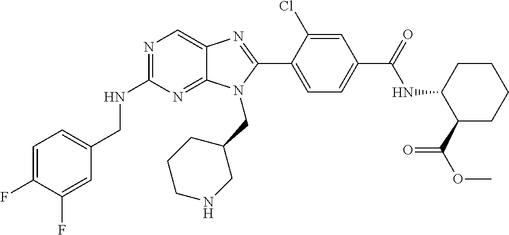
664 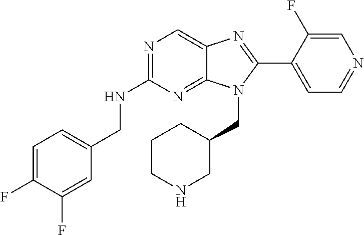
665 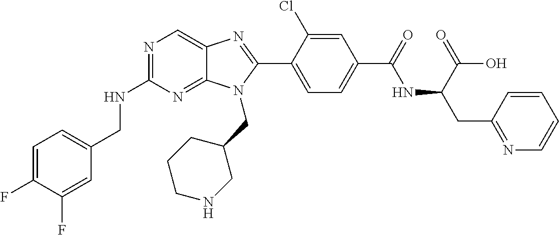
666 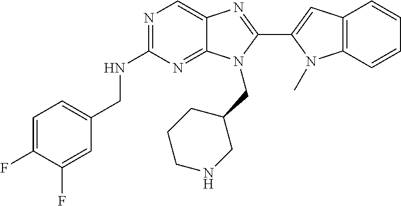
667 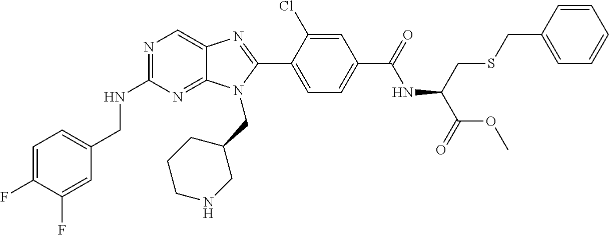
668 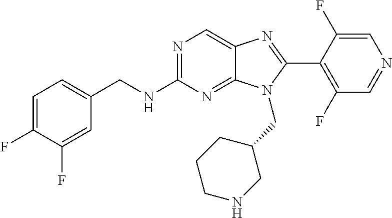
669 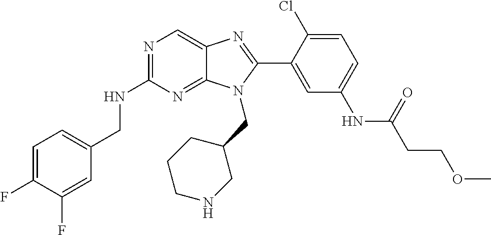
670 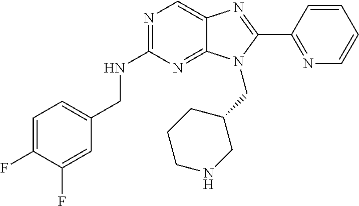
671 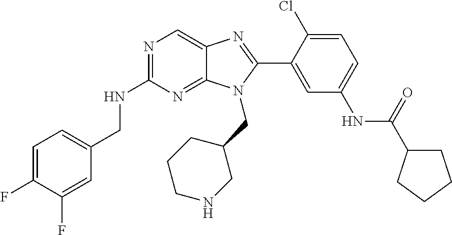
672 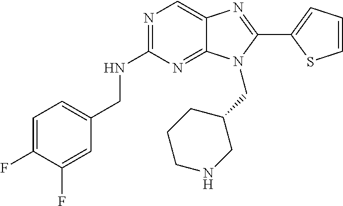
673 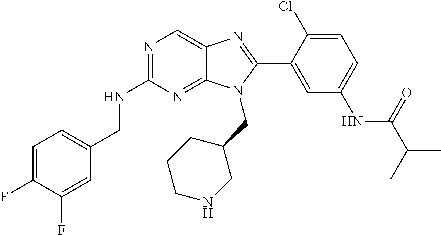
674 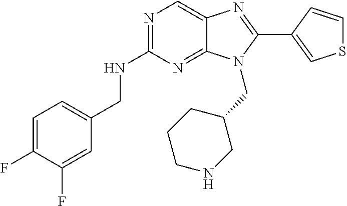
675 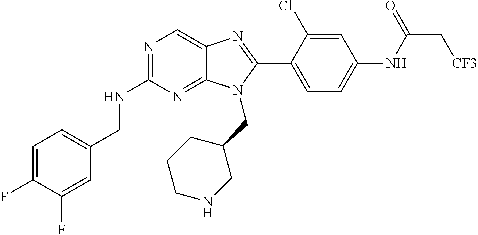
676 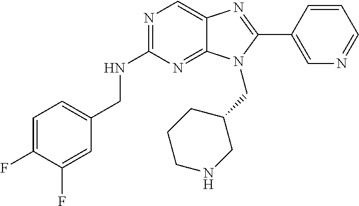
677 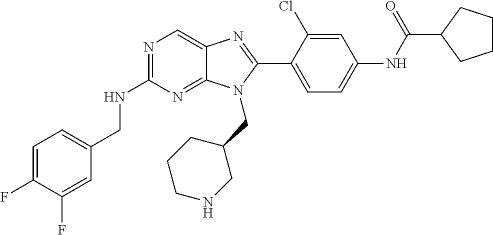
678 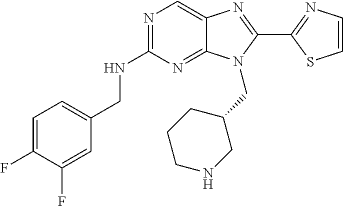
679 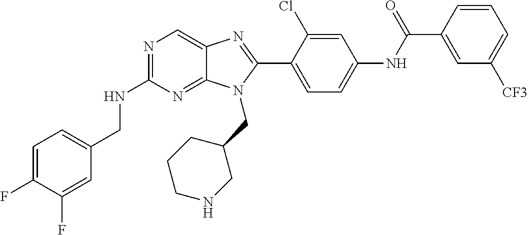
680 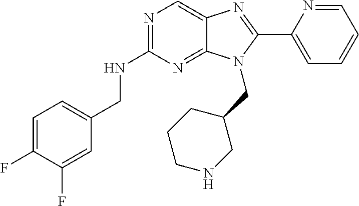
681 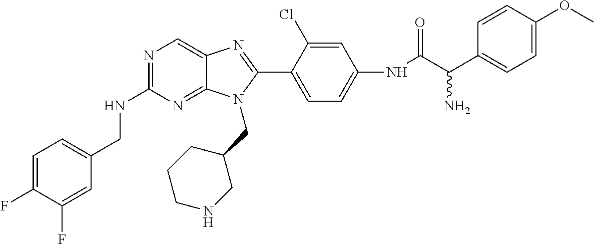
682 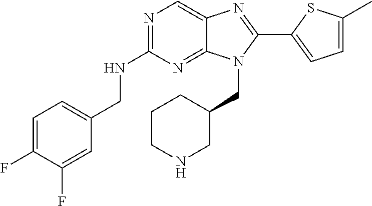
683 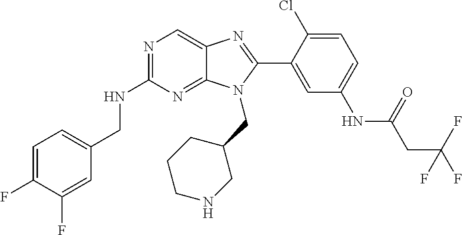
684 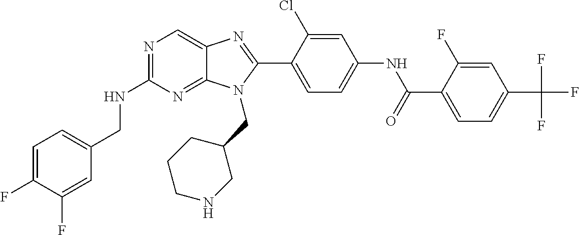
685 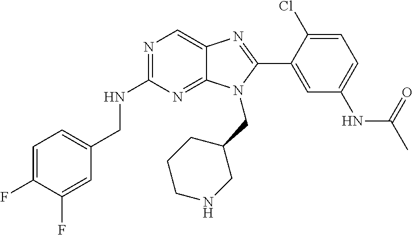
686 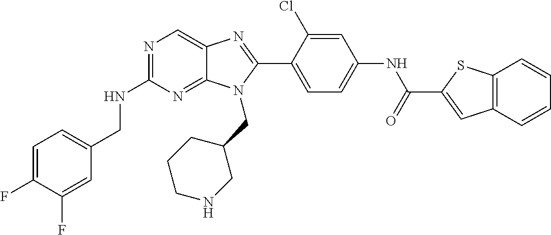
687 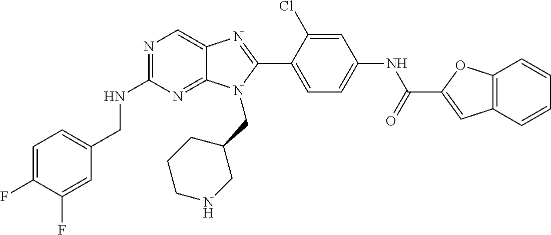
688 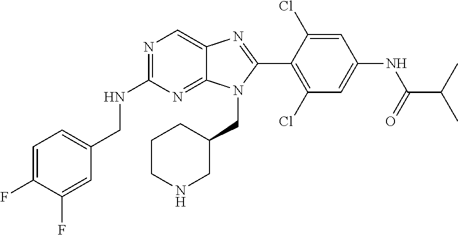
689 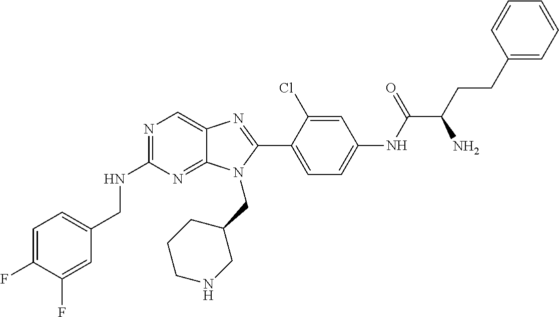
690 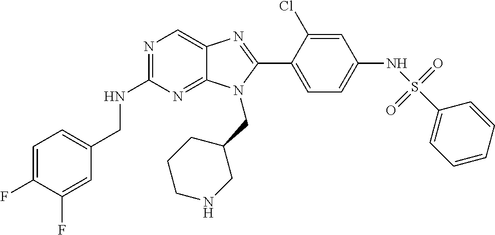
691 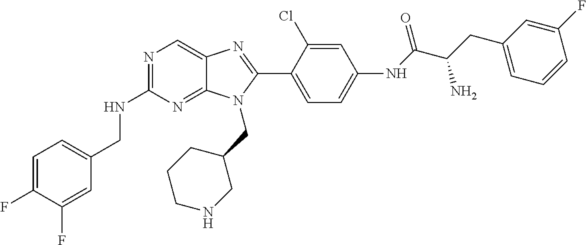
692 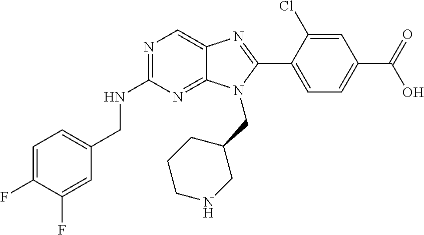
693 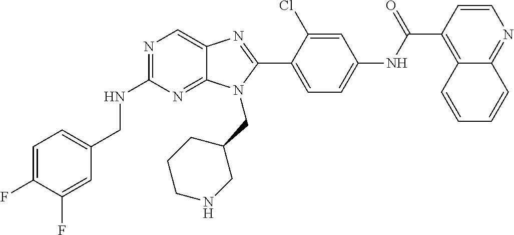
694 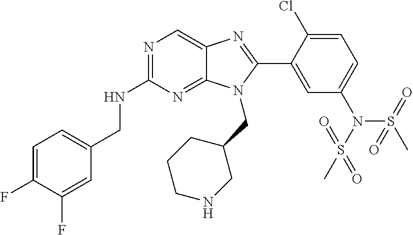
695 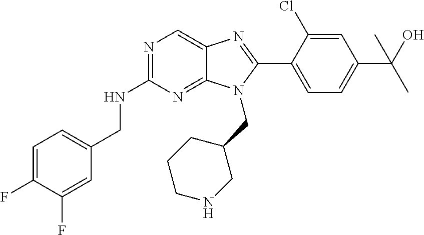
696 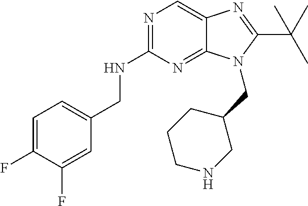
700 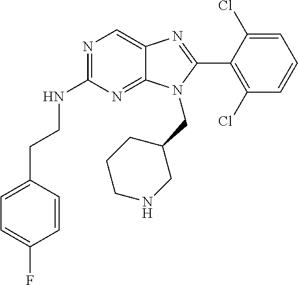
701 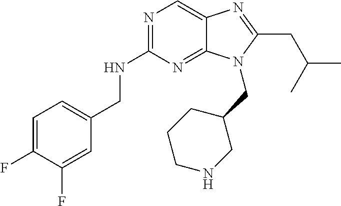
702 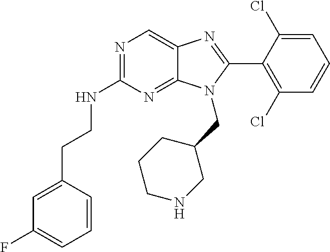
703 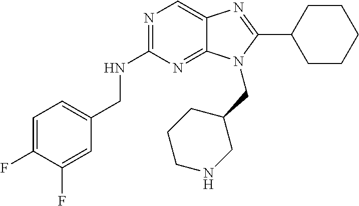
704 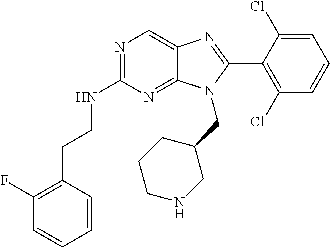
705 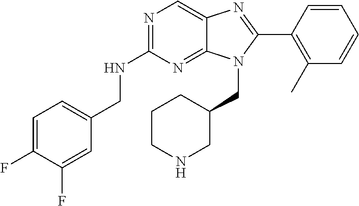
706 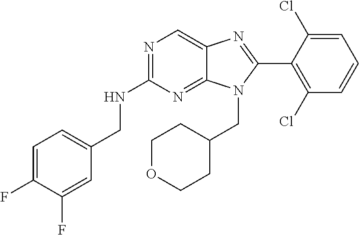
707 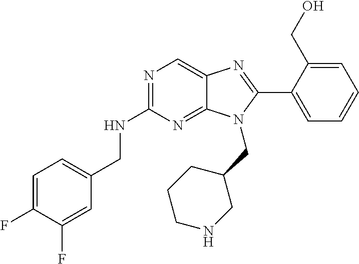
708 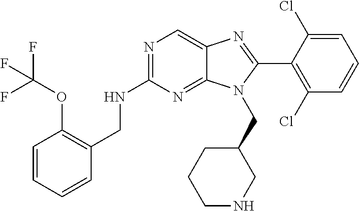
709 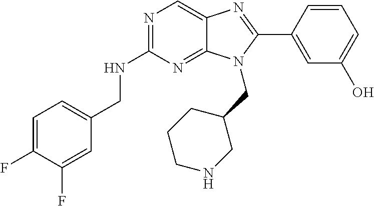
710 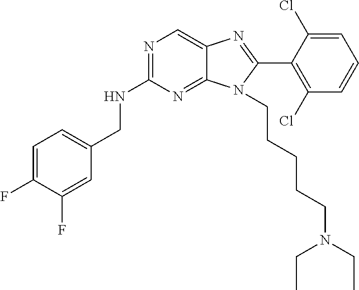
711 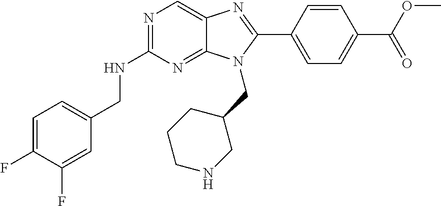
712 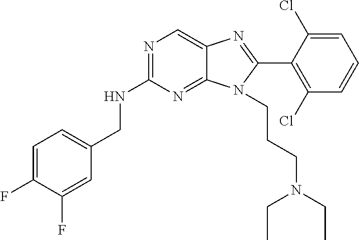
713 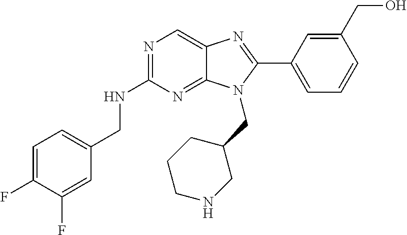
714 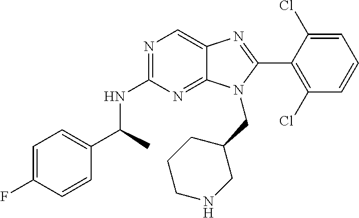
715 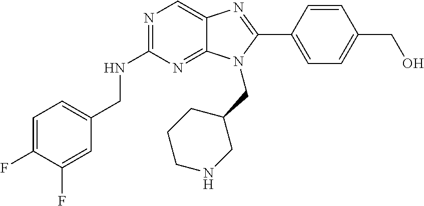
716 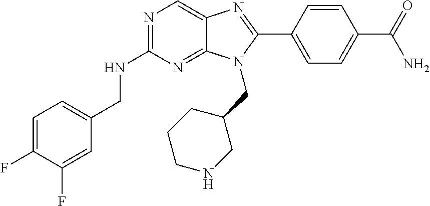
717 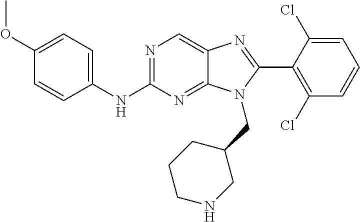
718 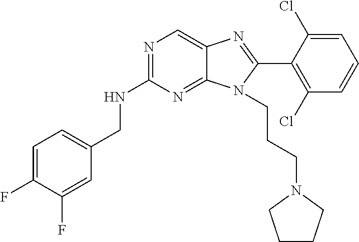
719 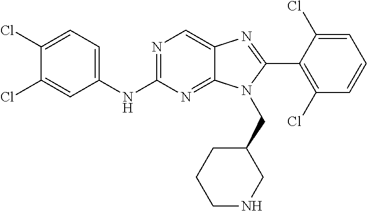
720 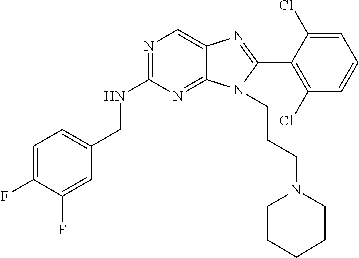
721 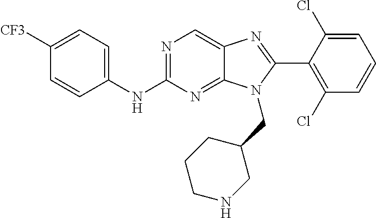
722 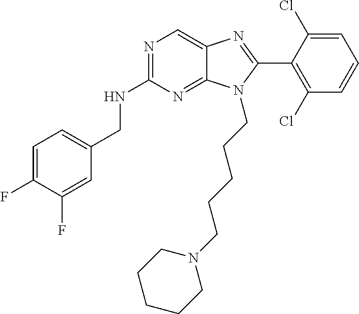
723 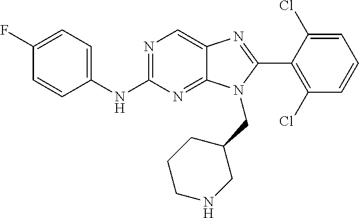
724 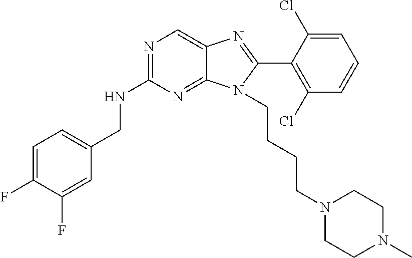
725 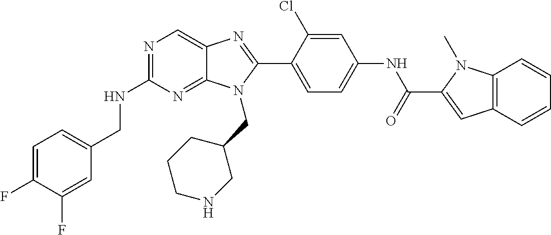
726 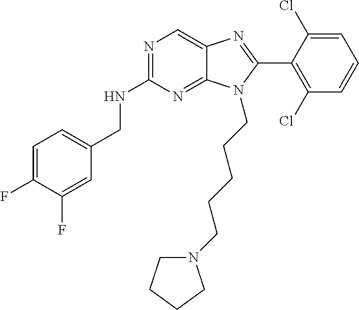
727 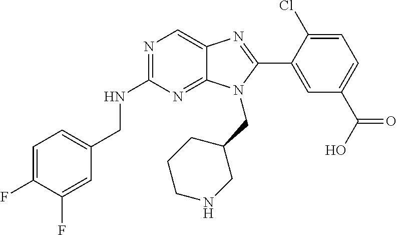
728 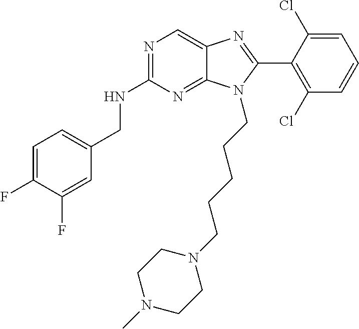
729

The data presented in Table 1 demonstrates utility of the compounds of the invention in inhibition of PKCθ. Therefore, the compounds of the invention are useful in treatment of T-cell mediated diseases including autoimmune disease such as rheumatoid arthritis and lupus erythematosus, and inflammatory diseases such as asthma and inflammatory bowel disease. Additionally, the compounds of the invention are useful in treatment of gastrointestinal cancer and diabetes.

Selectivity for inhibition of PKCθ by the compounds of the invention was tested and results are shown in Table 2. The data in Table 2 shows obtained values for PKCθ isoform selectivity by showing Ki Pan Vera (PV) potencies for PKCθ, PKC delta and PKC alpha. For Ki Pan Vera (PV) of PKCθ, entries identified with “A” had values below 100 nM; entries identified with “B” had values below 1 μM; and entries identified with “C” had values below 10 μM. For Ki Pan Vera (PV) of PKC delta and PKC alpha, entries identified with “1” had values above 250 nM; entries identified with “2” had values above 1 μM; entries identified with “3” had values above 10 μM.

Table 2 also shows selectivity of the compounds of the invention by showing their IC₅₀ values for SGK kinase. Entries identified with “1” had values above 250 nM; entries identified with “2” had values above 1 μM; entries identified with “3” had values above 10 μM.

TABLE 2

| | Ki PV | Ki PV-delta | Ki PV-alpha | IC50-SGK1 |
|---|---|---|---|---|
| Compound | (nM) | (nM) | (nM) | (nM) |
| 100 | A | 1 | 2 | Inactive |
| 101 | A | 1 | 2 | Inactive |
| 102 | A | 2 | 2 | 3 |
| 103 | A | 1 | 2 | |
| 104 | A | 1 | 2 | 3 |
| 105 | A | 1 | 2 | Inactive |
| 106 | B | 2 | 3 | Inactive |
| 107 | B | 2 | 3 | |
| 108 | B | 2 | 2 | Inactive |
| 109 | A | 2 | 2 | Inactive |
| 110 | A | 2 | 2 | 2 |
| 111 | A | 2 | 2 | Inactive |
| 113 | A | 1 | 2 | |
| 114 | | | | Inactive |
| 115 | B | 2 | 3 | 3 |
| 116 | B | 2 | 3 | Inactive |
| 117 | B | 2 | 2 | Inactive |
| 118 | B | 2 | 2 | 3 |
| 119 | A | 2 | 2 | |
| 120 | B | 2 | 2 | Inactive |
| 121 | A | 2 | 2 | Inactive |
| 122 | B | 2 | 3 | Inactive |
| 123 | B | 2 | 2 | |
| 124 | B | 2 | 2 | 3 |
| 125 | B | 2 | 2 | Inactive |
| 126 | B | 2 | 2 | 2 |
| 127 | B | 2 | 2 | Inactive |
| 128 | B | 2 | 2 | Inactive |
| 129 | B | 2 | 2 | Inactive |
| 130 | B | 2 | 2 | Inactive |
| 131 | B | 2 | 2 | Inactive |
| 132 | C | 2 | 3 | |
| 133 | B | 2 | 2 | Inactive |
| 134 | C | 2 | 3 | Inactive |
| 135 | B | 2 | 2 | Inactive |
| 136 | B | 2 | 2 | Inactive |
| 137 | | | | Inactive |
| 138 | B | 2 | 2 | Inactive |
| 139 | B | 2 | 2 | |
| 140 | B | 2 | 2 | |
| 141 | B | 3 | 3 | 2 |
| 142 | B | 2 | 2 | 3 |
| 143 | B | 2 | 2 | Inactive |
| 144 | B | 2 | 2 | Inactive |
| 145 | B | 2 | 2 | |
| 146 | B | 2 | 2 | Inactive |
| 147 | B | 2 | 2 | Inactive |
| 148 | B | 2 | 2 | 3 |
| 149 | B | 2 | | 3 |
| 150 | B | 2 | 2 | Inactive |
| 151 | B | 2 | 2 | 3 |
| 152 | B | 2 | 2 | |
| 153 | B | 2 | 2 | 2 |
| 154 | C | 2 | 2 | 2 |
| 155 | B | 2 | 2 | Inactive |
| 156 | B | 2 | 3 | 2 |
| 157 | B | 2 | 2 | |
| 158 | B | 2 | 2 | 3 |
| 159 | B | 2 | 2 | |
| 160 | B | 2 | 2 | Inactive |
| 161 | A | 1 | 1 | 3 |
| 162 | B | 3 | 2 | Inactive |
| 163 | B | 2 | 2 | Inactive |
| 164 | B | 2 | 2 | Inactive |
| 165 | C | 2 | 3 | |
| 166 | B | 2 | 2 | 3 |
| 167 | B | 2 | 3 | Inactive |
| 168 | B | 2 | 3 | Inactive |
| 169 | B | 2 | 3 | |
| 170 | B | 2 | 2 | Inactive |
| 171 | B | 2 | 2 | Inactive |
| 172 | B | 2 | 2 | Inactive |
| 173 | C | 2 | 2 | 3 |
| 174 | B | 2 | 2 | Inactive |
| 175 | B | 2 | | Inactive |
| 200 | C | 2 | 3 | Inactive |
| 201 | | | | Inactive |
| 203 | B | 2 | 3 | |
| 204 | B | 2 | 3 | |
| 205 | B | 2 | 2 | Inactive |
| 206 | C | 2 | 3 | 3 |
| 207 | C | 2 | 3 | Inactive |
| 209 | B | 2 | 2 | Inactive |
| 210 | C | 2 | 3 | Inactive |
| 211 | B | 2 | 2 | 2 |
| 212 | C | 3 | 3 | Inactive |
| 213 | C | 3 | 3 | Inactive |
| 214 | C | 2 | 2 | 3 |
| 215 | | | | Inactive |
| 216 | B | 2 | 2 | Inactive |
| 217 | B | 3 | 3 | Inactive |
| 220 | B | 2 | 2 | |
| 221 | | | | Inactive |
| 222 | | | | Inactive |
| 223 | B | 2 | 2 | 3 |
| 226 | B | 2 | 2 | |
| 238 | B | 2 | 2 | |
| 240 | B | 2 | 3 | Inactive |
| 262 | B | 2 | 2 | |

The compounds of the invention were also tested in vivo. Table 3 below demonstrates results of anti-CD3 induced interleukin-2 (IL-2) production in mice, which was performed following protocols disclosed in Goldberg et al. (2003), J. Med. Chem. 46, 1337-1349.

TABLE 3

| | Subcutaneous dose | % inhibition of |
|---|---|---|
| Compound | mg/kg | IL-2 production |
| Vehicle (no drug) | 0 | 0 |
| FK506 (positive control, | 1 | 87 |
| global immunosuppression) | | |
| 113 | 30 | 38 |
| 100 | 30 | 38 |
| 101 | 30 | 58 |
| 120 | 30 | 35 |

IL-2 is a T cell-derived lymphokine that modulates immunological effects on many cells of the immune system, including cytotoxic T cells, natural killer cells, activated B cells and lymphokine-activated cells. It is a potent T cell mitogen that is required for the T cell proliferation, promoting their progression from G1 to S phase of the cell cycle. It is a growth factor for all subpopulations of T lymphocytes, as well as stimulating the growth of NK cells. It also acts as a growth factor to B cells and stimulates antibody synthesis.

Due to its effects on both T and B cells, IL-2 is a major central regulator of immune responses. It plays a role in anti-inflammatory reactions, tumor surveillance, and hematopoiesis. It also affects the production of other cytokines, inducing IL-1, TNF-α and TNF-β secretion, as well as stimulating the synthesis of IFN-γ in peripheral leukocytes. IL-2, although useful in the immune response, also causes a variety of problems. IL-2 damages the blood-brain barrier and the endothelium of brain vessels. These effects may be the underlying causes of neuropsychiatric side effects observed under IL-2 therapy, e.g. fatigue, disorientation and depression. It also alters the electrophysiological behavior of neurons.

T cells that are unable to produce IL-2 become inactive (anergic). This renders them potentially inert to any antigenic stimulation they might receive in the future. As a result, agents which inhibit IL-2 production may be used for immunosupression or to treat or prevent inflammation and immune disorders. This approach has been clinically validated with immunosuppressive drugs such as cyclosporin, FK506, and RS61443.

The data presented in Tables 1-3 demonstrates utility of the compounds of the invention in inhibition of PKCθ and their utility for treatment of T-cell mediated diseases including autoimmune diseases such as rheumatoid arthritis, lupus erythematosus, and multiple sclerosis, inflammatory diseases such as asthma and inflammatory bowel disease, transplant rejection, gastrointestinal cancer, and diabetes.

Some of the compounds described herein contain one or more asymmetric centers and may thus give rise to enantiomers, diastereomers, and other stereoisometric forms which may be defined in terms of absolute stereochemistry as (R)- or (S)-. The present invention is meant to include all such possible diastereomers as well as their racemic and optically pure forms. Optically active (R)- and (S)-isomers may be prepared using homo-chiral synthons or homo-chiral reagents, or optically resolved using conventional techniques. When the compounds described herein contain olefinic double bonds or other centers of geometric asymmetry, and unless specified otherwise, it is intended to include both (E)- and (Z)-geometric isomers. Likewise, all tautomeric forms are intended to be included.

The graphic representations of racemic, ambiscalemic and scalemic or enantiomerically pure compounds used herein are taken from Maehr J. Chem. Ed. 62, 114-120 (1985): solid and broken wedges are used to denote the absolute configuration of a chiral element; wavy lines indicate disavowal of any stereochemical implication which the bond it represents could generate; solid and broken bold lines are geometric descriptors indicating the relative configuration shown but denoting racemic character; and wedge outlines and dotted or broken lines denote enantiomerically pure compounds of indeterminate absolute configuration. Thus, among the structures below, those having open wedges are intended to encompass both of the pure enantiomers of that pair, those having solid wedges are intended to encompass the single, pure enantiomer having the absolute stereochemistry shown.

The present invention includes compounds of formula (I) in the form of salts. Suitable salts include those formed with both organic and inorganic acids. Such salts will normally be pharmaceutically acceptable, although non-pharmaceutically acceptable salts may be of utility in the preparation and purification of the compound in question. The term “pharmaceutically acceptable salt” refers to salts prepared from pharmaceutically acceptable non-toxic acids or bases including inorganic acids and bases and organic acids and bases. When the compounds of the present invention are basic, salts may be prepared from pharmaceutically acceptable non-toxic acids including inorganic and organic acids. Suitable pharmaceutically acceptable acid addition salts for the compounds of the present invention include acetic, benzenesulfonic (besylate), benzoic, camphorsulfonic, citric, ethenesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric acid, p-toluenesulfonic, and the like. When the compounds contain an acidic side chain, suitable pharmaceutically acceptable base addition salts for the compounds of the present invention include metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine) and procaine.

While it may be possible for the compounds of formula (I) or their salts and solvates to be administered as the raw chemical, it is preferable to present them as a pharmaceutical composition. According to a further aspect, the present invention provides a pharmaceutical composition comprising a compound of formula (I) or a pharmaceutically acceptable salt or solvate thereof, together with one or more pharmaceutically carriers thereof and optionally one or more other therapeutic ingredients. The carrier(s) must be “acceptable” in the sense of being compatible with the other ingredients of the formulation and not deleterious to the recipient thereof.

The formulations include those suitable for oral, parenteral (including subcutaneous, intradermal, intramuscular, intravenous and intraarticular), rectal and topical (including dermal, buccal, sublingual and intraocular) administration. The most suitable route may depend upon the condition and disorder of the recipient. The formulations may conveniently be presented in unit dosage form and may be prepared by any of the methods well known in the art of pharmacy. All methods include the step of bringing into association a compound of formula (I) or a pharmaceutically acceptable salt or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Molded tablets may be made by molding in a suitable machine a mixture of the powdered compound moistened with an inert liquid diluent. The tablets may optionally be coated or scored and may be formulated so as to provide sustained, delayed or controlled release of the active ingredient therein.

Formulations for parenteral administration include aqueous and non-aqueous sterile injection solutions which may contain anti-oxidants, buffers, bacteriostats and solutes which render the formulation isotonic with the blood of the intended recipient. Formulations for parenteral administration also include aqueous and non-aqueous sterile suspensions, which may include suspending agents and thickening agents. The formulations may be presented in unit-dose of multi-dose containers, for example sealed ampoules and vials, and may be stored in a freeze-dried (lyophilized) condition requiring only the addition of a sterile liquid carrier, for example saline, phosphate-buffered saline (PBS) or the like, immediately prior to use. Extemporaneous injection solutions and suspensions may be prepared from sterile powders, granules and tablets of the kind previously described.

Formulations for rectal administration may be presented as a suppository with the usual carriers, such as cocoa butter or polyethylene glycol.

Formulations for topical administration in the mouth, for example buccally or sublingually, include lozenges comprising the active ingredient in a flavored basis such as sucrose and acacia or tragacanth, and pastilles comprising the active ingredient in a basis such as gelatin and glycerin or sucrose and acacia.

Preferred unit dosage formulations are those containing an effective dose, or an appropriate fraction thereof, of the active ingredient.

The pharmaceutical compositions will usually include a “pharmaceutically acceptable inert carrier” and this expression is intended to include one or more inert excipients, which include starches, polyols, granulating agents, microcrystalline cellulose, diluents, lubricants, binders, disintegrating agents, and the like. If desired, tablet dosages of the disclosed compositions may be coated by standard aqueous or nonaqueous techniques. “Pharmaceutically acceptable carrier” also encompasses controlled release means. Compositions of the present invention may also optionally include other therapeutic ingredients, anti-caking agents, preservatives, sweetening agents, colorants, flavors, desiccants, plasticizers, dyes, and the like.

The compounds of formula (I) are preferably administered orally or by injection (intravenous or subcutaneous). The precise amount of compound administered to a patient will be the responsibility of the attendant physician. However, the dose employed will depend on a number of factors, including the age and sex of the patient, the precise disorder being treated, and its severity. Also, the route of administration may vary depending on the condition and its severity.

The contents of each of the references cited herein, including the contents of the references cited within the primary references, are herein incorporated by reference in their entirety.

1. A compound, or salt thereof, represented by Formula I, 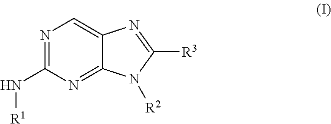
wherein:
R¹ is chosen from C₁-C₄ alkyl, carbocyclyl, substituted carbocyclyl, and 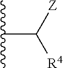
wherein
R⁴ is chosen from cycloalkyl, cycloalkylalkyl, aryl, arylalkyl, heteroaryl, and heteroarylalkyl, wherein R⁴ may be substituted, with a proviso that when R⁴ is a heteroaryl, R⁴ is not bonded via a heteroatom to the methylene carbon bearing the Z group; and
Z is chosen from —H and C₁-C₄ alkyl;
R² is chosen from —(C₂-C₇ alkyl)-NR⁵R⁶, —(C₀-C₄ alkyl)-R⁷-R⁸, and —(C₀-C₄ alkyl)-C(O)—(C₀-C₄ alkyl)-R⁷-R⁸,
wherein
R⁷ is cyclyl,
with a proviso that when R⁷ is a heterocyclyl, a purine nitrogen of Formula I bonded to R² is not bonded to a heteroatom of R⁷ directly or via a methylene group;
R⁸ is chosen from —(C₀-C₄ alkyl)-NR⁵R⁶, and
—C(O)—(C₀-C₄ alkyl)-NR⁵R⁶, and, when R⁷ is nitrogenous heterocyclyl, R⁸ may additionally be —H,
with a proviso that when R⁷ is a heterocyclyl and R⁸ is —(C₀-C₄ alkyl)-NR⁵R⁶, a heteroatom of R⁷ is not bonded to NR⁵R⁶ directly or via a methylene group;
R⁵ and R⁶ are independently chosen from —H and C₁-C₄ alkyl; and
R³ is chosen from C₁-C₆ alkyl, aryl, substituted aryl, arylalkyl, substituted arylalakyl, heteroaryl and substituted heteroaryl;
with a proviso that when R³ is phenyl and R² is piperidin-4-yl-ethyl, R¹ is not cyclopropyl.

2. A compound, or salt thereof, according to claim 1, wherein:
R¹ is chosen from C₁-C₄ alkyl, phenyl optionally substituted with one or two substituents independently chosen from halogen, OCH₃, —CF₃, —OCF₃ and C₁-C₄ alkyl 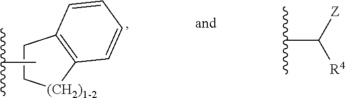
wherein
R⁴ is —(C₀-C₄ alkyl)-R⁹,
wherein
R⁹ is chosen from cycloalkyl, aryl, and heteroaryl, wherein R⁹ is optionally substituted at one or two atoms with substituents independently chosen from halogen, —OH, —OCH₃, —CF₃, —OCF₃, —CN, C₁-C₄ alkyl, and pyridinyl; and
Z is chosen from —H and C₁-C₄ alkyl.

3. A compound, or salt thereof, according to claim 1, wherein:
R² is chosen from —(C₂-C₇ alkyl)-NR⁵R⁶, —(C₀-C₄ alkyl)-R⁷-R⁸, and —(C₀-C₄ alkyl)-C(O)—(C₀-C₄ alkyl)-R⁷-R⁸,
wherein
R⁷ is chosen from alicyclyl, nitrogenous alicyclyl, aryl, and nitrogenous heteroaryl;
R⁸ is chosen from —H, —(C₀-C₄ alkyl)-NR⁵R⁶, and
—C(O)—(C₀-C₄ alkyl)-NR⁵R⁶; and
R⁵ and R⁶ are independently chosen from —H and —(C₁-C₄ alkyl).

4. A compound, or salt thereof, according to claim 1, wherein:
R³ is chosen from C₁-C₆ alkyl, aryl, aryl substituted with R¹⁰, R¹¹ and R¹²,
wherein
R¹⁰, R¹¹ and R¹² are independently chosen from —H, halogen, —OCH₃, —CF₃, —OCF₃, —CN, C₁-C₄ alkyl, —NR¹³R¹⁴, —S(O)ₘCH₃, —CONHR²², —NHCOR²³, —OR²⁴ and —NHS(O)ₘR²⁵;
wherein
R¹³ and R¹⁴ are independently chosen from —H and C₁-C₄ alkyl;
R²², R²³ and R²⁴ are one or two substituents independently chosen from —H, C₁-C₄ alkyl, C₁-C₆ cycloalkyl, aryl, —(CH₂)ₙNR²⁶R²⁷ and —(CH₂)ₙOR²⁸ said C₁-C₄ alkyl and C₁-C₆ cycloalkyl being optionally substituted with one or more halogens;
R²⁵ is C₁-C₄ alkyl;
R²⁶ and R²⁷ are independently chosen from H and C₁-C₄ alkyl or
R²⁶ and R²⁷ with the N to which they are attached form a 4-7 membered saturated heterocyclic ring optionally comprising an O;
R²⁸ is chosen from H and C₁-C₄ alkyl;
m is 0, 1 or 2 and
n is 1, 2 or 3.

5. A compound, or salt thereof, according to claim 1, wherein:
R¹ is chosen from C₁-C₄ alkyl, phenyl optionally substituted with one or two substituents independently chosen from halogen, OCH₃, —CF₃, —OCF₃ and C₁-C₄ alkyl 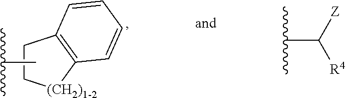
wherein
R⁴ is chosen from 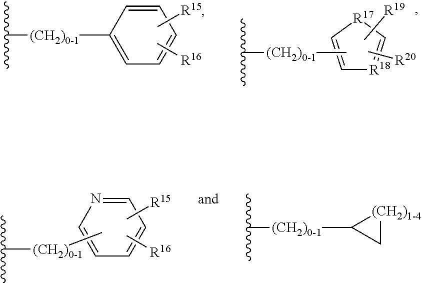
wherein
R¹⁵ and R¹⁶ are independently chosen from —H, halogen, —OH, —OCH₃, —CF₃, —OCF₃, —CN, C₁-C₄ alkyl, and pyridinyl;
R¹⁷ is chosen from O and S;
R¹⁸ is chosen from CH and N;
R¹⁹ and R²⁰ are independently chosen from —H, halogen, —OCH₃, —CF₃, —OCF₃, —CN, C₁-C₄ alkyl, and pyridinyl; and
Z is chosen from —H and C₁-C₄ alkyl.

6. A compound, or salt thereof, according to claim 1, wherein:
R² is chosen from —(C₂-C₇ alkyl)-NR⁵R⁶, —(C₀-C₄ alkyl)-R⁷-R⁸, and —(C₀-C₄ alkyl)-C(O)—(C₀-C₄ alkyl)-R⁷-R⁸,
wherein
R⁷ is chosen from cyclohexyl, phenyl, piperidinyl, pyrrolidinyl, morpholinyl, and piperazinyl;
R⁸ is chosen from —H, —(C₀-C₄ alkyl)-NR⁵R⁶, and —C(O)—(C₀-C₄ alkyl)-NR⁵R⁶; and
R⁵ and R⁶ are independently chosen from —H and —(C₁-C₄ alkyl).

7. A compound, or salt thereof, according to claim 1, wherein:
R² is other than

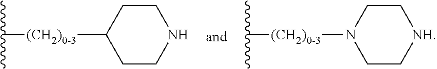

8. A compound, or salt thereof, according to claim 1, wherein:
R³ is chosen from C₁-C₆ alkyl, 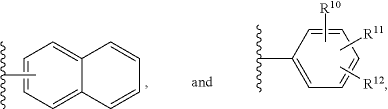
wherein
R¹⁰, R¹¹ and R¹² are independently chosen from —H, halogen, —OCH₃, —CF₃, —OCF₃, —CN, C₁-C₄ alkyl, —NR¹³R¹⁴, —S(O)ₘCH₃, —CONHR²², —NHCOR²³, —OR²⁴ and —NHS(O)ₘR²⁵;
wherein
R¹³ and R¹⁴ are independently chosen from —H and C₁-C₄ alkyl;
R²², R²³ and R²⁴ are one or two substituents independently chosen from —H, C₁-C₄ alkyl, C₁-C₆ cycloalkyl, aryl, —(CH₂)ₙNR²⁶R²⁷ and —(CH₂)ₙOR²⁸ said C₁-C₄ alkyl and C₁-C₆ cycloalkyl being optionally substituted with one or more halogens;
R²⁵ is C₁-C₄ alkyl;
R²⁶ and R²⁷ are independently chosen from H and C₁-C₄ alkyl or
R²⁶ and R²⁷ with the N to which they are attached form a 4-7 membered saturated heterocyclic ring optionally comprising an O;
R²⁸ is chosen from H and C₁-C₄ alkyl;
m is 0, 1 or 2 and
n is 1, 2 or 3.

9. A pharmaceutical composition comprising a compound, or salt thereof, according to claim 1, and a pharmaceutically acceptable carrier.

10. A compound, or salt thereof, represented by Formula I, 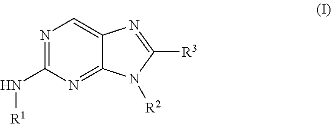
wherein:
R¹ is chosen from straight or branched C₁-C₄ alkyl, phenyl optionally substituted with one or two substituents independently chosen from halogen, OCH₃, —CF₃, —OCF₃ and C₁-C₄alkyl 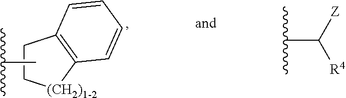
wherein
R⁴ is —(C₀-C₄ alkyl)-R⁹,
wherein
R⁹ is chosen from cycloalkyl, aryl, and heteroaryl,
wherein R⁹ is optionally substituted at one or two atoms with substituents independently chosen from halogen, —OH, —OCH₃, —CF₃, —OCF₃, —CN, C₁-C₄ alkyl, and pyridinyl,
with a proviso that when R⁹ is a heteroaryl, R⁹ is not bonded via a heteroatom to the methylene carbon bearing the Z group; and
Z is chosen from —H and C₁-C₄ alkyl;
R² is chosen from —(C₂-C₇ alkyl)-NR⁵R⁶, —(C₀-C₄ alkyl)-R⁷-R⁸, and —(C₀-C₄ alkyl)-C(O)—(C₀-C₄ alkyl)-R⁷-R⁸,
wherein
R⁷ is chosen from alicyclyl, nitrogenous alicyclyl, aryl, and nitrogenous heteroaryl,
with a proviso that when R⁷ is a nitrogenous alicyclyl or a nitrogenous heteroaryl, a purine nitrogen of Formula I bonded to R² is not bonded directly or via a methylene group to a nitrogen of R⁷;
R⁸ is chosen from, —(C₀-C₄ alkyl)-NR⁵R⁶, and —C(O)—(C₀-C₄ alkyl)-NR⁵R⁶, and, when R⁷ is nitrogenous alicyclyl or nitrogenous heteroaryl, R⁸ may additionally be —H,
with a proviso that when R⁷ is a nitrogenous alicyclyl or a nitrogenous heteroaryl and R⁸ is —(C₀-C₄ alkyl)-NR⁵R⁶, a nitrogen of R⁷ is not bonded directly or via a methylene group to —NR⁵R⁶; and
R⁵ and R⁶ are independently chosen from —H and —(C₁-C₄ alkyl);
R³ is chosen from C₁-C₆ alkyl, aryl, aryl substituted with R¹⁰, R¹¹ and R¹²,
wherein
R¹⁰, R¹¹ and R¹² are independently chosen from —H, halogen, —OCH₃, —CF₃, —OCF₃, —CN, C₁-C₄ alkyl, —NR¹³R¹⁴, —S(O)ₘCH₃, —CONHR²², —NHCOR²³, —OR²⁴ and —NHS(O)ₘR²⁵;
wherein
R¹³ and R¹⁴ are independently chosen from —H and C₁-C₄ alkyl;
R²², R²³ and R²⁴ are one or two substituents independently chosen from —H, C₁-C₄ alkyl, C₁-C₆ cycloalkyl, aryl, —(CH₂)ₙNR²⁶R²⁷ and —(CH₂)ₙOR²⁸ said C₁-C₄ alkyl and C₁-C₆ cycloalkyl being optionally substituted with one or more halogens;
R²⁵ is C₁-C₄ alkyl;
R²⁶ and R²⁷ are independently chosen from H and C₁-C₄ alkyl or
R²⁶ and R²⁷ with the N to which they are attached form a 4-7 membered saturated heterocyclic ring optionally comprising an O;
R²⁸ is chosen from H and C₁-C₄ alkyl;
m is 0, 1 or 2 and
n is 1, 2 or 3.

11. A compound, or salt thereof, according to claim 10, wherein:
R¹ is chosen from C₁-C₄ alkyl, phenyl optionally substituted with one or two substituents independently chosen from halogen, OCH₃, —CF₃, —OCF₃ and C₁-C₄ alkyl 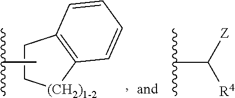
wherein
R⁴ is chosen from 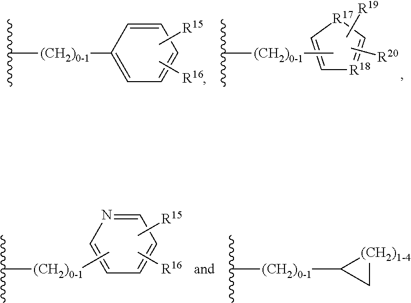
wherein
R¹⁵ and R¹⁶ are independently chosen from —H, halogen, OH, —OCH₃, —CF₃, —OCF₃, —CN, C₁-C₄ alkyl, and pyridinyl;
R¹⁷ is chosen from O and S;
R¹⁸ is chosen from CH and N;
R¹⁹ and R²⁰ are independently chosen from —H, halogen, —OCH₃, —CF₃, —OCF₃, —CN, C₁-C₄ alkyl, and pyridinyl; and
Z is chosen from —H and C₁-C₄ alkyl.

12. A compound, or salt thereof, according to claim 10, wherein:
R² is chosen from —(C₂-C₇ alkyl)-NR⁵R⁶, —(C₀-C₄ alkyl)-R⁷-R⁸, and —(C₀-C₄ alkyl)-C(O)—(C₀-C₄ alkyl)-R⁷-R⁸,
wherein
R⁷ is chosen from cyclohexyl, phenyl, piperidinyl, pyrrolidinyl, morpholinyl, and piperazinyl;
R⁸ is chosen from —H, —(C₀-C₄ alkyl)-NR⁵R⁶, and —C(O)—(C₀-C₄ alkyl)-NR⁵R⁶; and
R⁵ and R⁶ are independently chosen from —H and —(C₁-C₄ alkyl).

13. A compound, or salt thereof, according to claim 10, wherein:
R² is other than

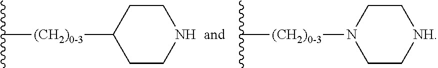

14. A compound, or salt thereof, according to claim 10, wherein:
R³ is chosen from C₁-C₆ alkyl, 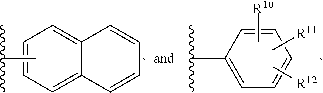
wherein
R¹⁰, R¹¹ and R¹² are independently chosen from —H, halogen, —OCH₃, —CF₃, —OCF₃, —CN, C₁-C₄ alkyl, —NR¹³R¹⁴, S(O)ₘCH₃, —CONHR²², —NHCOR²³, —OR²⁴ and —NHS(O)ₘR²⁵;
wherein
R¹³ and R¹⁴ are independently chosen from —H and C₁-C₄ alkyl;
R²², R²³ and R²⁴ are one or two substituents independently chosen from —H, C₁-C₄ alkyl, C₁-C₆ cycloalkyl, aryl, —(CH₂)ₙNR²⁶R²⁷ and —(CH₂)ₙOR²⁸ said C₁-C₄ alkyl and C₁-C₆ cycloalkyl being optionally substituted with one or more halogens;
R²⁵ is C₁-C₄ alkyl;
R²⁶ and R²⁷ are independently chosen from H and C₁-C₄ alkyl or
R²⁶ and R²⁷ with the N to which they are attached form a 4-7 membered saturated heterocyclic ring optionally comprising an O;
R²⁸ is chosen from H and C₁-C₄ alkyl;
m is 0, 1 or 2 and
n is 1, 2 or 3.

15. A compound, or salt thereof, according to claim 10, wherein:
R¹ is 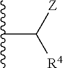
wherein
R⁴ is chosen from and 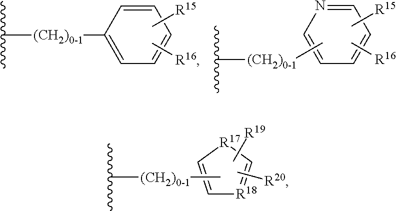
wherein
R¹⁵ and R¹⁶ are independently chosen from —H, halogen, —OH, —OCH₃, —CF₃, —OCF₃, —CN and C₁-C₄ alkyl;
R¹⁷ is chosen from O and S;
R¹⁸ is chosen from CH and N;
R¹⁹ and R²⁰ are independently chosen from —H, halogen, —OCH₃, —CF₃, —OCF₃, —CN, C₁-C₄ alkyl, and pyridinyl; and
Z is chosen from —H and C₁-C₄ alkyl.

16. A compound, or salt thereof, according to claim 10, wherein:
R² is chosen from

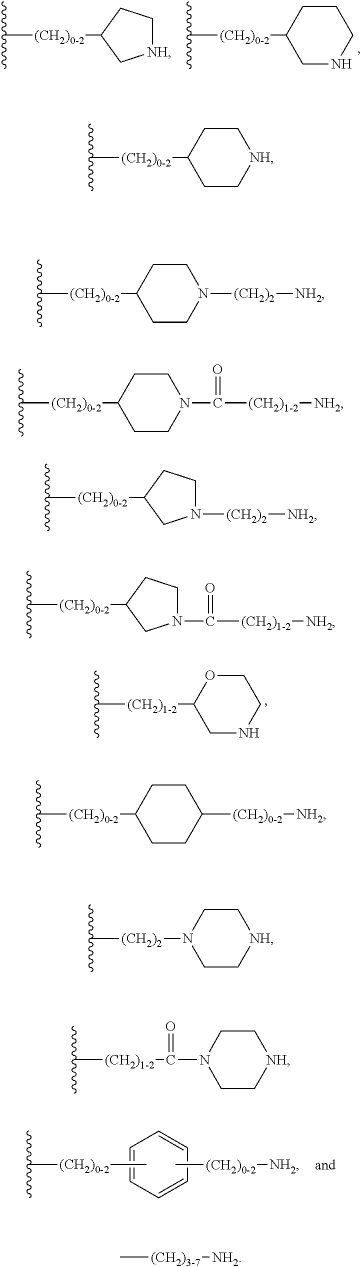

17. A compound, or salt thereof, according to claim 10, wherein:
R² is chosen from 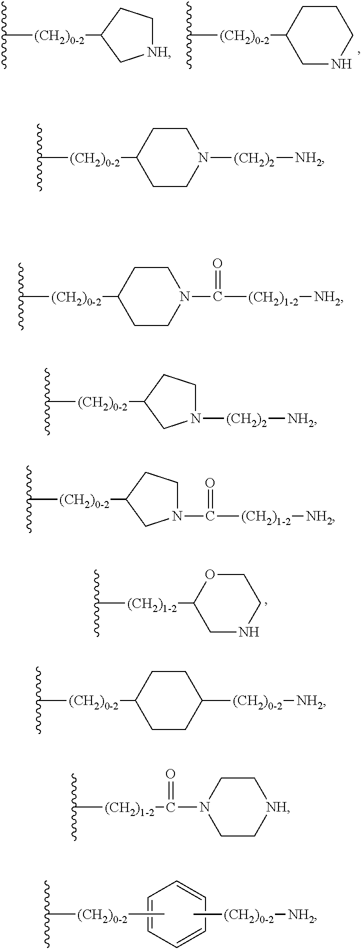
and —(CH₂)₃₋₇—NH₂.

18. A compound, or salt thereof, according to claim 10, wherein:
R³ is 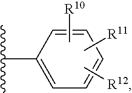
wherein
R¹⁰, R¹¹ and R¹² are independently chosen from —H, halogen, —OCH₃, —CF₃, —OCF₃, —CN, C₁-C₄ alkyl, —NR¹³R¹⁴, —S(O)ₘCH₃, —CONHR²², —NHCOR²³, —OR²⁴ and —NHS(O)ₘR²⁵;
wherein
R¹³ and R¹⁴ are independently chosen from —H and C₁-C₄ alkyl;
R²², R²³ and R²⁴ are one or two substituents independently chosen from —H, C₁-C₄ alkyl, C₁-C₆ cycloalkyl, aryl, —(CH₂)ₙNR²⁶R²⁷ and —(CH₂)ₙOR²⁸ said C₁-C₄ alkyl and C₁-C₆ cycloalkyl being optionally substituted with one or more halogens;
R²⁵ is C₁-C₄ alkyl;
R²⁶ and R²⁷ are independently chosen from H and C₁-C₄ alkyl or
R²⁶ and R²⁷ with the N to which they are attached form a 4-7 membered saturated heterocyclic ring optionally comprising an O;
R²⁸ is chosen from H and C₁-C₄ alkyl;
m is 0, 1 or 2 and
n is 1, 2 or 3.

19. A pharmaceutical composition comprising a compound, or salt thereof, according to claim 10, and a pharmaceutically acceptable carrier.

20. A compound, or salt thereof, represented by Formula I, 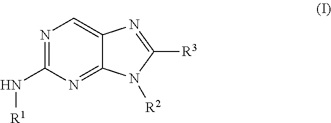
wherein:
R¹ is chosen from C₁-C₄ alkyl, phenyl optionally substituted with one or two substituents independently chosen from halogen, OCH₃, —CF₃, —OCF₃ and C₁-C₄ alkyl 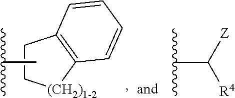
wherein
R⁴ is chosen from 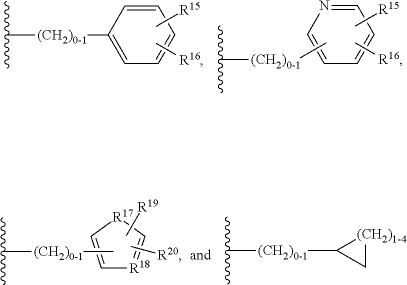
wherein
R¹⁵ and R¹⁶ are independently chosen from —H, halogen, —OH, —OCH₃, —CF₃, —OCF₃, —CN, C₁-C₄ alkyl, and pyridinyl;
R¹⁷ is chosen from O and S;
R¹⁸ is chosen from CH and N;
R¹⁹ and R²⁰ are independently chosen from —H, halogen, —OCH₃, —CF₃, —OCF₃, —CN, C₁-C₄ alkyl, and pyridinyl; and
Z is chosen from —H and C₁-C₄ alkyl;
R² is chosen from —(C₂-C₇ alkyl)-NR⁵R⁶, —(C₀-C₄ alkyl)-R⁷-R⁸, and —(C₀-C₄ alkyl)-C(O)—(C₀-C₄ alkyl)-R⁷-R⁸,
wherein
R⁷ is chosen from cyclohexyl, phenyl, piperidinyl, pyrrolidinyl, morpholinyl, and piperazinyl;
R⁸ is chosen from —H, —(C₀-C₄ alkyl)-NR⁵R⁶, and —C(O)—(C₀-C₄ alkyl)-NR⁵R⁶; and
R⁵ and R⁶ are independently chosen from —H and —(C₁-C₄ alkyl);
and R² contains a basic N atom located from 2 to 8 atoms distant from its point of attachment to the purine ring;
R³ is chosen from C₁-C₆ alkyl, 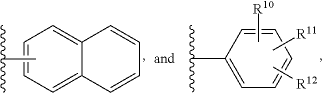
wherein
R¹⁰, R¹¹ and R¹² are independently chosen from —H, halogen, —OCH₃, —CF₃, —OCF₃, —CN, C₁-C₄ alkyl, —NR¹³R¹⁴, —S(O)ₘCH₃, —CONHR²², —NHCOR²³, —OR²⁴ and —NHS(O)ₘR²⁵;
wherein
R¹³ and R¹⁴ are independently chosen from —H and C₁-C₄ alkyl;
R²², R²³ and R²⁴ are one or two substituents independently chosen from C₁-C₄ alkyl, C₁-C₆ cycloalkyl, aryl, —(CH₂)ₙNR²⁶R²⁷ and —(CH₂)ₙOR²⁸ said C₁-C₄ alkyl and C₁-C₆ cycloalkyl being optionally substituted with one or more halogens;
R²⁵ is C₁-C₄ alkyl;
R²⁶ and R²⁷ are independently chosen from H and C₁-C₄ alkyl or
R²⁶ and R²⁷ with the N to which they are attached form a 4-7 membered saturated heterocyclic ring optionally comprising an O;
R²⁸ is chosen from H and C₁-C₄ alkyl;
m is 0, 1 or 2 and
n is 1, 2 or 3.

21. A compound, or salt thereof, according to claim 20, wherein:
R¹ is 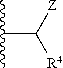
wherein
R⁴ is chosen from and 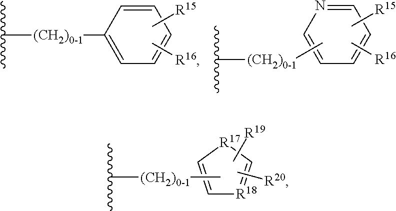
wherein
R¹⁵ and R¹⁶ are independently chosen from —H, halogen, —OH, —OCH₃, —CF₃, —OCF₃, —CN and C₁-C₄ alkyl;
R¹⁷ is chosen from O and S;
R¹⁸ is chosen from CH and N;
R¹⁹ and R²⁰ are independently chosen from —H, halogen, —OCH₃, —CF₃, —OCF₃, —CN, C₁-C₄ alkyl, and pyridinyl; and
Z is chosen from —H and C₁-C₄ alkyl.

22. A compound, or salt thereof, according to claim 20, wherein:
R² is not

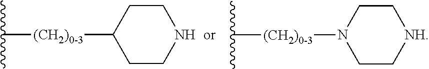

23. A compound, or salt thereof, according to claim 20, wherein:
R² is chosen from 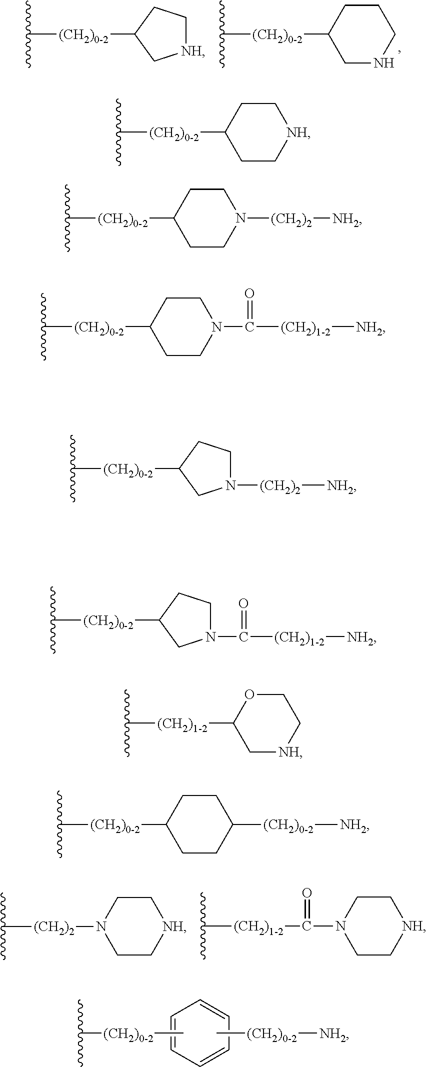
and —(CH₂)₃₋₇—NH₂.

24. A compound, or salt thereof, according to claim 20, wherein:
R² is chosen from 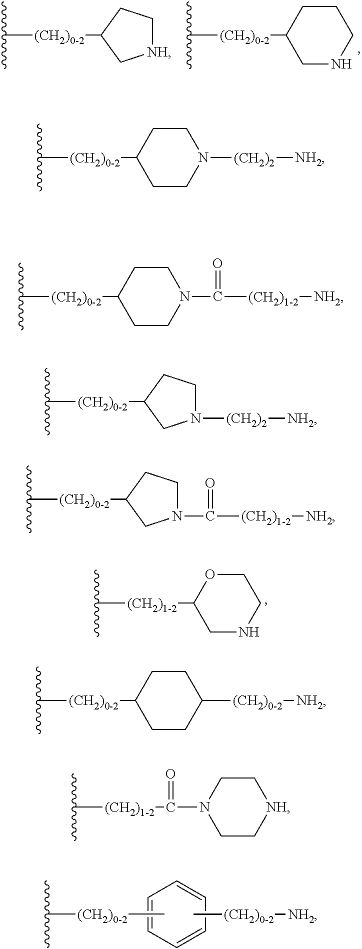
and —(CH₂)₃₋₇—NH₂.

25. A compound, or salt thereof, according to claim 20, wherein:
R³ is 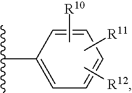
wherein
R¹⁰, R¹¹ and R¹² are independently chosen from —H, halogen, —OCH₃, —CF₃, —CN, C₁-C₄ alkyl, —NR¹³R¹⁴, —S(O)ₘCH₃, —CONHR²², —NHCOR²³, —OR²⁴ and —NHS(O)ₘR²⁵;
wherein
R¹³ and R¹⁴ are independently chosen from —H and C₁-C₄ alkyl;
R²², R²³ and R²⁴ are one or two substituents independently chosen from —H, C₁-C₄ alkyl, C₁-C₆ cycloalkyl, aryl, —(CH₂)ₙNR²⁶R²⁷ and —(CH₂)ₙOR²⁸ said C₁-C₄ alkyl and C₁-C₆ cycloalkyl being optionally substituted with one or more halogens;
R²⁵ is C₁-C₄ alkyl;
R²⁶ and R²⁷ are independently chosen from H and C₁-C₄ alkyl or
R²⁶ and R²⁷ with the N to which they are attached form a 4-7 membered saturated heterocyclic ring optionally comprising an O;
R²⁸ is chosen from H and C₁-C₄ alkyl;
m is 0, 1 or 2 and
n is 1, 2 or 3.

26. A pharmaceutical composition comprising a compound, or salt thereof, according to claim 20, and a pharmaceutically acceptable carrier.

27. A method of treatment of a T-cell mediated disease in a patient in need thereof, the method comprising administering to the patient a therapeutically effective amount of a compound, or salt thereof, of claim 1.

28. The method of claim 27 wherein the T-cell mediated disease is an autoimmune disease.

29. The method of claim 28 wherein the autoimmune disease is rheumatoid arthritis.

30. The method of claim 28 wherein the autoimmune disease is lupus erythematosus.

31. The method of claim 28 wherein the autoimmune disease is multiple sclerosis.

32. The method of claim 27 wherein the T-cell mediated disease is an inflammatory disease.

33. The method of claim 32 wherein the inflammatory disease is asthma.

34. The method of claim 32 wherein the inflammatory disease is inflammatory bowel disease.

35. The method of claim 27 wherein the T-cell mediated disease is transplant rejection.

36. A method of treatment of cancer in a patient in need thereof, the method comprising administering to the patient a therapeutically effective amount of a compound, or salt thereof, of claim 1.

37. The method of claim 36 wherein the cancer is gastrointestinal cancer.

38. A method of treatment of diabetes in a patient in need thereof, the method comprising administering to the patient a therapeutically effective amount of a compound, or salt thereof, of claim 1.

39. A method of treatment of a T-cell mediated disease in a patient in need thereof, the method comprising administering to the patient a therapeutically effective amount of a compound, or salt thereof, of claim 10.

40. A method of treatment of a T-cell mediated disease in a patient in need thereof, the method comprising administering to the patient a therapeutically effective amount of a compound, or salt thereof, of claim 20.

41. A method of treatment of cancer in a patient in need thereof, the method comprising administering to the patient a therapeutically effective amount of a compound, or salt thereof, of claim 10.

42. A method of treatment of cancer in a patient in need thereof, the method comprising administering to the patient a therapeutically effective amount of a compound, or salt thereof, of claim 20.

43. A method of treatment of diabetes in a patient in need thereof, the method comprising administering to the patient a therapeutically effective amount of a compound, or salt thereof, of claim 10.

44. A method of treatment of diabetes in a patient in need thereof, the method comprising administering to the patient a therapeutically effective amount of a compound, or salt thereof, of claim 20.
